SEQUENCE LISTING (94 sequences)

SEQ ID NO: 1 - DNA, 615 bp, White spot syndrome virus
  source 1..615: mol_type = genomic DNA
atggatctttctttcactctttcggtcgtgtcggccatcctcgccatcactgctgtgattgctgtatttattgtgatttttaggtatcacaacactgtgaccaagaccatcgaaacccacacagacaatatcgagacaaacatggatgaaaacctccgcattcctgtgactgctgaggttggatcaggctacttcaagatgactgatgtgtcctttgacagcgacaccttgggcaaaatcaagatccgcaatggaaagtctgatgcacagatgaaggaagaagatgcggatcttgtcatcactcccgtggagggccgagcactcgaagtgactgtggggcagaatctcacctttgagggaacattcaaggtgtggaacaacacatcaagaaagatcaacatcactggtatgcagatggtgccaaagattaacccatcaaaggcctttgtcggtagctccaacacctcctccttcacccccgtctctattgatgaggatgaagttggcacctttgtgtgtggtaccacctttggcgcaccaattgcagctaccgccggtggaaatcttttcgacatgtacgtgcacgtcacctactctggcactgagaccgagtaa

SEQ ID NO: 2 - DNA, 407 bp, White spot syndrome virus
  source 1..407: mol_type = genomic DNA
tgacgtacctcttcatcaaacagaaaaaaaatggccaccacgactaacactcttcctttcggcaggaccggagcccaggccgctggcccttcttacaccatggaagatcttgaaggctccatgtctatggctcgcatgggtctctttttgatcgttgctatctcaattggtatcctcgtcctggccgtcatgaatgtatggatgggaccaaagaaggacagcgattctgacactgataaggacaccgatgatgatgacgacactgccaacgataacgatgatgaggacaaatataagaacaggaccagggatatgatgcttctggctgggtccgctcttctgttcctcgtttccgccgccaccgtttttatgtcttaccccaagaggaggcagtaaaaatataaaaa

SEQ ID NO: 3 - DNA, 1242 bp, White spot syndrome virus
  source 1..1242: mol_type = genomic DNA
atggagtcaatcaaactgttcaccgttgctggtctgaatatggagcaagccaaccaagtggctgaagaaatcaagtcagaatataaaaccgaggaggaaaagaggattgcccaggaagtgtttgacaaattcaccaaaaaactcattatgcaagtagatacgtttaaacacttacttacaagagaaaaccccaaccgttttgtatcccgccccattgtccatgaagatctctgggaaatgtacaaaaaagaggttgcctgtttttggacattggaagagattgatttcgaaagggatcctaaagattgggagaaactcactcaagatgagaaggatttcattctccagattctggcgttctttgcatcctctgacggaattgtaattgaaaatcttacaacacgtcttcgtcaagtggcgcagattccagaagcgaggagtttctttgacttccaagttggaatggagagtattcatggcaacgtctacggagaactgattgatagactggtgcccgacgaaaaagacaaggctatcttgtttaacgctgcacaacacttccccgccatcaagaagaaggagcagtgggctattaattggatgcaaagcaataacgatttggcggaactaattgttgcctttgctgcagttgaaggaatcttctttagtggtgcattcgcatccattttctggatcaagaacaggggtattttgcctggtctcacctcctccaatgagttcatttctagggacgaaggtcttcatcgcgactttgcatgcatgctgttgaaaaagggttttgttgataccccatcaagagaaaggattcttgaaattgtcactgaagccgtccgaattgaacaagaatttctcacagtttccctgcctgttaaattagtgggaatgaactgcaagttgatgagccagtacattgaatttgtggcagataaactattggttgaaatgggactagaaaagcactataatgttaccaaccccttcccattcatggacaatatttccctcgagaataagaccaacttttttgaaaagagagtcgccgagtatcaacgtgcccaggtcatggcttctatcaataagatcaagaaggaccaacaaacccaagaaactggttctcctctcccaattctgactgcacctcctccagtctcttcctcatcatccgaacaagaagatgttgaagacggcgtcggggactacatcagttatgacgatttttag

SEQ ID NO: 4 - DNA, 790 bp, White spot syndrome virus
  source 1..790: mol_type = genomic DNA
ctcatgccggagaagtgattgaaggttgtactacagtttcttctatcaaggaactaatcagtgtgttaccagaagttgtggatgtgattctcattgacgaagggcaattcttcacggatttggtgctagtcaatagactggctgacaaggggaaaaggattgtgattgcagcacttgatggaacttctgaccagcaaatgttcagtcctattcataagctattgccttatacaaattccattgttaagctagcatctaaatgtatgatttgtaaaattgataccaaagaagctccttttactgtaaggtttggtaatgacaatgataataatgttatatgtgtaggaggagctgaaatgtacgctgctgcctgccgggactgttacaaaaaaattaacaagaaaaagaacaaggggaaacttgttgtacttgaaggaggtgacaggtgcggtaagagtacccaagccaaactcttgttgaccaataaaaactcgcctctttatggaggagaatacatgtgctttcccgacaggagcagccatacgggtaaactcatcaatgattatttaactaagaaaattgaactagatgatcatgcagctcacttgttattttctgcaaatagatgggaagtttgtagtaaaattaagcagttgttagacgatggaatccatgttgtgatggatagatattactactcggggattgttttctctttagctagaggagtggataccgttgagtggtgctctgctagcgatgagggacttcctcagcccgatcttgtatt

SEQ ID NO: 5 - DNA, 805 bp, White spot syndrome virus
  source 1..805: mol_type = genomic DNA
cggcggtggacaagaggttggttgaatctctaaagaaaactcacaattccacattcgtaattatttaaattgggattagatcgaataatgtatatcttcgtcgaaggttcccccctcacagggaagagttcatggatgtccaagttgatagatacaggatcatgtggaatgtctttcctcaattttcttcgtatgaacacttctgactactacaactggcctgccgaaatcgggacagaacatctccagttaggtttcagagaaaccagagtggtggatggaatgtttgaacctgtcctaaagacctttgtcgactcgtggaagaaagagcaaggaaaagagagtttgaaggaatatctggactacaacggccaagtcatggagatctacatcgcagaatggttgagacaaaggccactagccttccacgtgtttacctatacagatgaagctgtcaagagtggattcttgaacgaggaggatctagatatggatactgcaaccaagtggatggctgaaattattagagagaagaggggcaatattcaagaaataaaagtgacccctagagtagtcttcaatggcaatggttgtagtgcatgtttctctaacactaagagaaacttgtataactttggaacaaactataacaatgttgtacattgtgatttgttgtgcccttttgcaaggcataggattgtacatttcttataatggtttaaggaacgtgttttcgagtcttccttttacaaaataaaacaatatataaaattcaaaaaaaaaaaaaaaaaaaaaaaaaaaaaaa

SEQ ID NO: 6 - DNA, 615 bp, White spot syndrome virus
  source 1..615: mol_type = genomic DNA
atggaatttggcaacctaacaaacctggacgttgcaattattgcaatcttgtccattgcaatcattgctctaatcgttatcatggttataatgattgtattcaacacacgtgttggaagaagcgtcgtcgctaattatgatcagatgatgcgagtcccaattcaaagaagggcaaaggtaatgtcaattcgtggagagaggtcctacaatactcctcttggaaaggtggccatgaagaatggtctctccgataaggacatgaaggatgtttctgctgatcttgtcatctctaccgtcacagccccaaggactgatcccgctggcactggggccgagaactctaacatgactttgaagatcctcaacaacactggcgtcgatctcttgatcaacgacattactgttcggccaactgttattgcaggaaacattaagggaaatactatgtcgaacacttacttctcgagcaaggacattaaatcttcatcttcaaaaattaccctcattgacgtgtgcagcaaatttgaagacggcgcagccttcgaagctacaatgaacattggattcacctccaggaatgtgatcgatatcaaggacgaaatcaagaagaagtaa

SEQ ID NO: 7 - DNA, 1164 bp, Taura syndrome virus
  source 1..1164: mol_type = genomic DNA
tcaaaagatagggatatgacgaaagtcaacgcatatgagaacttacctggtaagggcttcactcatggtgttggcttcgattatggcgtacccctgtctcttttccctaacaatgccattgatcccacaattgcagtgcctgaaggattggatgagatgtctattgaatacttagcacagcgaccatatatgctcaacagatacactatcagaggtggtgacactcctgatgcgcatggaacaattattgcagatattccagtgagtcctgtcaattttagtttgtatggtaaagttattgctaagtatcgcaccctattcgctgccccagttagtctagctgtagcaatggctaattggtggcgtggaaatattaaccttaatcttcgctttgctaagacgcagtaccatcaatgcagattgctggtgcaatatctcccctatggtagtggtgttcaaccaatagaaagtatcctttcacagatcatcgacatctcacaagtcgatgataagggtattgacattgcttttccttccgtctatcccaataagtggatgcgagtgtacgatccagcgaaagttgggtacacggcagattgtgccccaggccgaatcgtcatttccgttctcaatccacttatctcagcttcgacagtctctcctaatattgtcatgtatccttgggtgaattggagcaatttagaggttgctgaaccaggtacgcttgctaaagcagccatcggcttcaattatccagcagatgttcctgaggagcccactttttcagtaacgcgtgctccagtatctggaacactgtttacgttactccaggatacgaaggtgtctttgggggaagctgacggtgtattctcattatactttacgaacactaccactggtggaaggcacagactagcttatgccggactgcctggtgaactcggtagttgtgagatagtgaagctacctcaagggcaatattcaattgaatacgcagctaccagcgctccaactcttgttctcgatagacctatcttttctgaaccgattggccccaagtatgtagtcactaaagttaagaacggtgatgttgttggtatttccgaggagacgttagtaacatgtggtagtatggcagcgattggtgaagctacggtcgcattg

SEQ ID NO: 8 - DNA, 1274 bp, Taura syndrome virus
  source 1..1274: mol_type = genomic DNA
gttcggattttcaaaagatagggatatgacgaaagtcaacgcatatgaaaatttacctggtaagggcttcacccatggtgttggctttgattatggtgtacccctgtctcttttccccaacaatgccattgatcccactattgcagtgcccgaaggattggatgaaatgtctattgaatacctagcacaacgaccatatatgctcaacagatacactatcagaggtggtgacacccctgatgcgcatggcactattatcgcggatattccagtgagtcctgttaattttagcctgtatggcaaagttattgctaagtatcgcaccctattcgctgccccaattggtctagcagtagcaatggctaattggtggcgaggtaacatcaacctcaatcttcgctttgctaagacgcagtaccatcaatgtaggttgctggtgcaatatctcccatatggtagtggtgttcaaccgatagaaagtatcctttcagagattattgacatttcacaagtcgatgataagggtattgacgtcgctttcccttccgtctatcctaataagtggatgcgagtgtacgatccagcgaaagttggctacacggcagattgtgccccaggccgcatcgtcatttccgttcttaatccactcatttcagcttcaacagtctctcctaatattgtcatgtatccttgggtgacttggagcaatttaggggttgctgaaccaggtacgcttgctaaggcagccattggcttcaactatccagcggatgttcccgaggagccaactttttcagtgacgcgtgctccagcgtctggaacactgtttactgtgctccaggatacgaaggtgtctttgggggaagctgacggtgtattctcattatcctttacgaacaccaccagtgacaaaaaatacaaactagcatatgctggcctgcctggtgaacttggtagttgtgaaatagtagagttacctcaagggcaatattcaattgaatatgcagctactagcgccccaactcttgttttaaatagacctatcttttctgaaccacttggccctaagtatgtagttactaaagtaaagaatggtgatgttattagtatatccgaggagacgttagtaacatgtggtaaaatgtcagcagttggccaagctacggttgcattgcgaggctcagatgcgccaattgaaattttgaaattagcttcagattttgagtcctcagctccagtgaagttcagtccaggtgactttaccgtagtcactg

SEQ ID NO: 9 - DNA, 859 bp, Yellow-head Virus
  source 1..859: mol_type = genomic DNA
ctcagtgactacacatcattcatcagatcacaaatcgaacacatcaaacaccacttctctatctggctatttgaggtgattccaaagatctccatccaacctgtagacaaagcactgcgttccatcttcatcggcccagccttcatgaacgatgtttaccgctgcttcaacaccgcatggctagaattcaccaaaacacgactttcctacaacactgtcctcatcggattcaaagacacacactgtggcatcaacaagctgatcaacgggatcaaggcaggattcaacccaaaaggcaaagccaaatggatctcgcaagactatcccaagtttgacacctgtgttgacaccatggcacaatactcttacatcatgaatcacgcatatcactacactcacactaacctctctcttattactcgtggcctctgtcagctaattgcaaactccaccagcccaatcatctactacaacagcatgctcatcaggaaactgcatggggtgtcaagtggtgacggcgcaacagcaattaagaacagccactgtaacagtatcatcacaaacatcgccttctacagacaaatcattgacaatcaagtgtccgaagaataccgcggtctacaatccactctctacaacacactcatcaatggcatacaaagtaaagacgatgcatactctacacaccgcgctttcgaatggaacatctctcgctgtgccactcttagcgacgacacacttgccatcatcaatccagacgtcttcgatcttgatcaatacctttcaagctatcgaactctaggtggttatgaaatcactaatgagaaaaagatcttcgtccgtgatgagccatatgaat

SEQ ID NO: 10 - DNA, 2539 bp, Yellow-head Virus
  source 1..2539: mol_type = genomic DNA
tctcttgccgcacgcaaacgatacatcatccatgctctgagtgtgagaatggacatcaggaaaatgtatgtagacaacgacttccgctgcgcccagcatctagacaaggaaggttattctaagatcactgacgacaagaacaacgtcaccatctttagaactaacgcaacccgttcagaacttcctggcgtaagcaagattctcaagcaagtcgaaaagaaaagcttcttcaagatcatctatgaattcacactcaagcactggatccaacacaacatcctcctcagtttcattgccgctcgtgaagacatgccatctgaagtcatcgatttcaaagtgataaagcaagacggtgacaagacttacatcaagacagacaaattcttctcattcaagatcaacaacaaatacattgacgctacaacacttcaagacatttcactcaagcaagaatacaccaatggtaaactctcactatacatcgatggcggtatcaagaagggtatcaagaacatcagaatcaccactcctaatccttccaactctcttaccctctggaatactatcactcctactactacagtcctcaacatcttccgtggcattcaattccaagcttccaccttctacattgacaacaccggaagtcttcttgacaagatggccaataacaacatcaccgtcattcaagggccacctggtaccggtaagacatacaccataaacaaattccttgcaagaatcaatgaaattctccctaattccaacattgccgtccttgctagctcccactctgcagttgataacattggtaactcagtcaatccctccatcttccgcagatgcaagagacttatccctcaagaatcagaagacaaagtccgtaccaggttccaaagatacacaactggcggtggcatcatcttcgctactctccagtcaacccgtggtattttatgtcctagtgtagaatacatgatcatcgatgaattctcaatggccactgacattcaaatctactccgccatctgtcatctcaatccacgtcatgtcatcttcactggtgatccttgtcaactatccactcaacacgtatacaacacagacgtttaccattccaacatcatcaacaatcatcagctcacaggcaagttccctactgaattccttgatatcacctatcgcatgggctcaaagatcaatgatttcatctcagaaaacttctacaacggtaagctcaaaacagccgctacgtatgaaggtgaggtcttccagctccacctagaccctatcaacatgctctcacagatccatgcaatttgggaatcatcacctcagaacaactttgccattctagttacacatcatgaagcattctccatcatcagacaatatttcactgaccttgacattcaaatccccatctacactgtccacacatcgcaaggcagaacatttgaccgtggaatcgtcgtcagctaccgcaacaccgcgttcacaaaggatccaaacattgtgaatgtagccgtcagtcgtttccgcttccagtgtatctgcatgcaccagggcaatccatactacgctaaacttccatactacaacacatcacaaatctactttgagaaatccactacagttatcgcatacaatggaccacagaacaaactctccaacatgtacactgacaacatcaagccatttccataccacacccttgaaaaccgctatcagagtgaaaaggcaaagtatcttggcaagaaattaatcctccacaacaatccatttgaaactctcaaagaagccaagaaagtgttcacacgcgaagataacctcagatgggccaaagtatcagcagaagtaatgactcgcttgctgttcgagaaattcaacaatcccgatctagctaagcatctcatcaacactggcaaaagtcatctagtcgaaaacaccaagcatcctatctggggcggcaaaggtggtgaaaaccttcacgggaaaatcctcaccaacatccgtaccaaattggaagtccgcgaacgtgaacccactctgatcgacacttcatacaaacacaatgtcatcttccacaaattcaagaatcaaatcatatcagctccacgtctagacattcacgaaaacacactgtgcattgatgttgaaactgtaaacagtaaagatatcaaaggtaccgacaacaagagcctccattttccatcacaaattggctttgcttacaacggcaccattgaaacatacgattgcactcctacactcacagtcaatggacactcaatcactaccaaatacttcaactctatctatccttcttctgtcaaggccgccaaacgtggaaagcacaagattgaattcattctccgctcctacatggctcgcctccaacacactgtcactgatcaaatcaccctcgtctttaaatcagcactcatcgatgtctcaatgatccacaacgcaatcagaaatggaccaaacaaatgtcacaatgaagattgcgcaaaccatcccatctggtacacagagc

SEQ ID NO: 11 - DNA, 1005 bp, Yellow-head Virus
  source 1..1005: mol_type = genomic DNA
tccaccgacgacaagtactcctactatcaacagctcgacttcttcgtccacgccgcattcctcaacgcatacgcccaagcaattgactcaaagatcccactcaacttcactcaccgtaacctgaacttccgtgtttcacagcccaagacaaacttccttgtcggtctcgtcactcatgaagtaaacacaggcaacaacaccagagtagaagatctcaacaagcacccatacaacaagtaccgtagtaatatcgtacgtgtatatggtgaacgtggtgatctcaacggcttcctcagcggcaaattcctctacttccctcgtcacatcttcgactcctgtactgacaacactctcacacgacacatccgcgtcacaaaaggtgaagaaactcatgacatcgaactgttgagcgacgaatatgacgccactcctttcatcaaaatcgacagtccattcgcagaagcaactgtactcaaattcggtaaactccaacgtacccagtacgcatacttcgttaccgctgatgacatcagggttggttcaatgtccgtcgacggctaccacaacatttctaccaaggatggtgactgcggttcactcctctttgaccaccttcacaatgttgttggagctcacatcgtcggcattgctagcatccctcctgtcaacggtgccctgacctggaatgcagaaaaggaaatgctctgcggaccaaatgatgactacgattacgatccagaaaaagtcggtccacccaaggtatggcctgtagaatcaatcactgctctcagcacgatcctcaatcagctcaactatgtcaccggtgatgccttcactacacccaaactccctacaaactatcagctaattggttgcgagacactggaccagtacgtcaatgcccgtaatctcgttaccggccaatttcctcagatcaaggaggcactagacgatttcatcaacggatacgtcgccaacttgcaacgaggtactgaagcctacaac

SEQ ID NO: 12 - DNA, 231 bp, Gill-associated virus
  source 1..231: mol_type = genomic DNA
agggtaatccatactactctaaacttccttactacaacacagcacagatttacttcgagaaatcaaccacagtcatcgcttataacgggcccaacaacaaactctcaaacatgtacactgataatctcaaaccattcccataccatactcttgagaaccgttaccagagcgagaaggctaagtatctcggtaagaaattaatcttgcataacaatccattcgagacactta

SEQ ID NO: 13 - DNA, 1278 bp, Gill-associated virus
  source 1..1278: mol_type = genomic DNA
cacttctcatcctttcgctcccagccattttcatcttctactgggctgcggggcttatttctgaccttcctcttatctactttactctctggttctggggttcatggcttcttgctacaatttgctactccctcacattctctggacgcaggcgcaacaaggacaaattctattattctatcaacgtgcaacaacacaatccgtacaataaaagaatcttgtcatctaagttcgcttaagtctgaggtctcctgcttttacgatgaacacattcatgacatttctgcaccaggctcgcatatcatttatacaattggttttgcttacctgaaaacagattatgaatgtaaagaaggatatcctcccgccaacaccgtccaacgatacttccttgctattttataccttagcatctgttctcttctagcctacattttcactaaaatcactcctatcgctaaatccttcatctcttcatgctttcaaactgaatatatcatggaaacgatcgaaactgaaaaaggccaagtgactgttgcccatgatagacataagctcacaccaacaagttgccattctattctcaactccactttcaatcttctagttattggatgtttcatatttctatgtttcttcatcactcctgctttcgccacgattctaagtggaattcctgaaaaagacaagtcagtcctcatggctccccacgtactttttgaggccggccaaccaactgagcctccagactgcatccattgggctgctaatggtgactgtttctgcaactctaccaactgcgactggagtgaacacgttcaaactctttgtcctcaaacctgtaacacatcatctcccactacaacttcttctgccacacaatctcttccgtcttccactccttctagtgacgccgataatccttgtgttgcacaggacgatgccggttgttacagctatcttaatgactatgacgaatcaaaacgtacacaagccattaaatacacatacacactcagcacaaagaatacaccacatatgaatgcaatcctaactactgagcaatttgaagaaatgtctttagatgaagcaagatactctgacattgtatctttataccgaatcaataacataacatcagtccctggctgtatgtacaatccagtatcatactatctccatggcgactctgttcctgttacttgtccatcaacaccaagatctttcggtacaacctacaatcatcaaatcagtagtcaaattttgtataatcacaaa

SEQ ID NO: 14 - DNA, 748 bp, Yellow-head Virus
  source 1..748: mol_type = genomic DNA
tccagaaggcgtctatgacttcgagacataccgacccggcacctgcgatcccatcatagctctcaatgccgtcacgtattgtatcgaacgtcactggttctccgctggcctctcactctcatgcgcctcaatctacccacacgaagacatgacaattcaccagtacaaagaagcattcgcatcctacactacagaattaaacacagaagtcacactcaaacaccaaccaacattcgcctcatacctcactttcatgcttgtcaacgcacgtcacaaaatcgacatcgacatcggcacaggcccagacaccttctacacatcattcgacaacatcacatccgctccatgcacagacgaacgatacaacgaagtcatgacagggattactcgcttatattatgcataccagtatgatagaggtgattttccttgcaaatacacagtcactcaaacacacatcaaataccctgtaattggcgatgtccctgtggaacctgaagaatgcaaagacctcacttgcaacagttacccgccagtatatggtgccctgatctccatccagaagttcagcacttgggcccgcctcttgtgctacgacgttctcaaaagggttttcaaacactgccgtaactgcgaccacctcaattgcaagatctcaagacagctcacgcgcttcaaaaatccactctccaacatccaaccagtcgcatacacaaaactacacgacgaccgttacctaatccgtgac

SEQ ID NO: 15 - DNA, 842 bp, Yellow-head Virus
  source 1..842: mol_type = genomic DNA
catgattgctgtcttcgatgctcagtttccaacatagccttccagatctgtgaccctgccaccaatgaatgcgcatccatcacaacagattatagaaattacaccgcagacttcaagacaattagttttaacatcgctcatccccaagtagctaagacaacattcaaagttggtgcgcttttcacccatggtacaacccatcccaagcatctcctctacgatgttcctggcaagtacgatgctccacctggttctttctttagctatcaagctgagactgttcccaccggtgcattttgtaacaagaacgcatggatgtcccctggcctagcatcaacagacattaaatacaatggcttcccaacattagattataaactaaattttgcaactataaatcaagccattcgcagctacgaccccttaggagctatagttcaatgtaattacgatcagacatatatttccactatcacagcccgtcagcaacgttcacttcttctaaacggagttacttacacagatgaggtagatgctctttcgtatagtctcctcctgaatccgcatcactgcgatttcggagccgtgaacatacacttcgcatattccactactgctcatgtttctctaatagaaggtgaccctgaccaactctctttcgactgtacaggctgtcttttcactaacaaccaaatgcaatgttccatcaaaggtattgactctcactcttatcaaattactgattctcttaatactttcggcgctgcaacatgctctcatactaaagccgatcatctctgtaatttcactgcttccgcatctgagttcgatctaaagttaat

SEQ ID NO: 16 - DNA, 417 bp, Yellow-head Virus
  source 1..417: mol_type = genomic DNA
actcctatgcctcgtcgtcgcctacctccttccaaccgacccactcgcaatgcaaggctcatcgagattcctcaatccttcgcagtcgaacgcggaaatggatggatgttggcatatgccccaggtaaaaatccactaccgggaaaagtcatcgctcgtatgcaggcatctccattcattcaaggacttcaagaacaatccctccaagttgtcaagtcatctgatggtaagtattcaatttcaaagagatacggtaaaatggccatcacttatcttaatcccaacgatcccattctgccaaagcgttcaacacagaagtcaatcgttcccgatccttcccttgacatagagaacctagctgaaggtatccacgcaatgagccttgaagacgacgaatccatggaaacacaatcataa

SEQ ID NO: 17 - DNA, 1629 bp, Yellow-head Virus
  source 1..1629: mol_type = genomic DNA
tgtccattccacaccaccaagaatgacacgtgcatgatctgcgcaaacatcgacgacggcatctactctgtcgttccagccgattcatcattccacgagctcgccagggagctttccatcactctcactaatgtcaccaatcctcctcctaagagacatgaggcaaattttcagacaaagcctcgtcgagtcttccagtaaaccgctaccactacatcaactcagccccgttcaactacacaatcaccaattcatcacctggtgatggctgggttgagtttactggggacaatcacgaggcttgtgtcggccataagatctgggccaagaaatattaccacaccagtggaatattctgtaaacctcgagcttcatcatccactctcatcatcaatgggaagaaccgacattacaagactaaacactcactcaaacgcgaaatcaaaactgcgcaggcactctcacacattccagaagctgtccaattccacaaggatgagcatgggtactatcgtgaaatctcacaattctcccttgctgatgtgttgcatgggttcgctaatcaaatcgaaccagacttcctagccaaatacaccaacgagcgtaacatcacagtctcgaatgtaacttggctctgcaagaacatctgtaatcacaaatcatgcaacatcctcaagatggacatcttcgactacacctacacctgctacacaaaagcacagtcattcgcattacaagcatgtacaatatacaacttcgacatcactccagacaacatctgtccagaaggcgtctatgacttcgagacatatcgtcccggcaattgtgatcccatcaaagctctcaacgccgtcacgtattgcatcgaacgtcactggttctccgctggtctctcactctcctgcgcatctatttacccacatgaagacatgacaattcatcagtacaaagaagcattcgcactctacactacagaattgaacacagaagtcactctcaaacaccaacctactttcgactcctatctcaacttcatgcttgtcacagagcgtcacaacatcaacattgacatcggcacaggagcagacaccttctacacttccttcgacaacatcacatcagctccgtgtacagaagaacgatacaacgaagtcatggccggggttactcgcttatattatgcctaccagtatgatagaggtgattttccttgcaaatacacagtcactcaaactcacgtcaaataccctgtaattggcgatgttgctgtggaacctgaagaatgcaaaaatccgatctgcaacagttacccaccagtctacagtgctctgatctccatccagaaattcagcacctgggctcgtctcatgtgtcatgatattctcaagcgagtcttcaatcactgtcgtgactgtgaccatcttaactgcaagatctcacggcaactcatgcgcttcaagaatccactttccaacattcagccagtcgcatacactaaactacacgaagatcgtcacctaatccgtgaccgtctcacacacatggacttcacctcaggtcaagagttctacgctactgaattcatcaatgagttcaatgacgtcgaattcaagacactgaacggtgata

SEQ ID NO: 18 - DNA, 5001 bp, Yellow-head Virus
  source 1..5001: mol_type = genomic DNA
atgcaatgttcgcgatcattcaccatgcacttctcatcctttcgctcccagccattttcatcttctactgggctgcggggcttatttctgaccttcctcttatctactttactctctggttctggggttcatggcttcttgctacaatttgctactccctcacattctctggacgcaggcgcaacaaggacaaattctattattctatcaacgtgcaacaacacaatccgtacaataaaagaatcttgtcatctaagttcgcttaagtctgaggtctcctgcttttacgatgaacacattcatgacatttctgcaccaggctcgcatatcatttatacaattggttttgcttacctgaaaacagattatgaatgtaaagaaggatatcctcccgccaacaccgtccaacgatacttccttgctattttataccttagcatctgttctcttctagcctacattttcactaaaatcactcctatcgctaaatccttcatctcttcatgctttcaaactgaatatatcatggaaacgatcgaaactgaaaaaggccaagtgactgttgcccatgatagacataagctcacaccaacaagttgccattctattctcaactccactttcaatcttctagttattggatgtttcatatttctatgtttcttcatcactcctgctttcgccacgattctaagtggaattcctgaaaaagacaagtcagtcctcatggctccccacgtgctttttgaggccggtcaaccaactgagctttcagactgcatccattgggctgctaatggtgactgtttctgcaactctaccaactgcgactggagtgaacacgttcaaactctttgtcctcaaacctgtaacacatcatctcccactacaacttcttctactacacaatctcttccgtcttccactccttctagtgacgccgataatccttgtgttgcacaggacgatgccggttgttacagctatcttaatgactatgacgaatcaaaacgtacacaagccattaaatacacatacacactcagcacaaagaaaacaccacatatgaatgcaatcctaactactgagcaatttgaagaaatgtctttagatgaagcaagatactctgacattgtatctttataccgaatcaataacataacatcagtccctggctgtatgtacaatccagtatcatactatctccatggcgactctgttcctgttacttgtccatcaacaccaagatctttcggtacaacctacaatcatcaaatcagtagtcaaattttgtataatcacaaaatggtaaatgttacagtagatcaacgttgcaaaacacattctgataactgttgggcttattataacaaggcatcgaaaagcatttttatccaattccatcccagctatgctcagaagtatcataatagaatattagagcccactacattaattattccattctatcctcccagagacactaaaacactagccacacatcttggtcctcgcgttcttcgtaatgccggtgattatcaaattttccttgaacctggctggctcggtagaacataccttgacggttattcttatcatgaaatttatgcatccacacgtcacgattgccgttgcaacatgatgtccggcgacaataagtatggtattaacctcggagatgacgtccttcatgagaccattccaactccccgcggttatactcctagcgttgttgtctgtggcactacatttacatattataaattacatgacgcagttaaactcccatgggaaagcgttcaatataccgatatagaggacattcctgccggtttccgtgatccctatgacttctccgtcgatactccatctgggcctgtaactatcagtgttctggaagagtatcatgacggagattccatacaggagacggctcctaagcgtttcttcatctactatcggataatgacagcccgtcttacaccctcacaagtcgaacatcttaatctatctacacacgccacaagttcctgggcagctgaaaattacataagtaactgctacgttgttagacagcaattcgtcagaaacactcacccattctcatttgctctttcttacattgactacaatgtaacagctggtagtgttgttcgatgcaacgaattcaacatacaaatggatcttcttcttgcaacctttggtatagcaacacgtacgtgggcggctgagtatcgtcatctgccacattttctcaccaagcgcggtttctatcccctcgaacctgttaccggttctgcaatagattacttaatagtcgaatacaacgcccatgcttcacgttactcacatcaggcaacttaccatcaatttggacatccagttgctaaagctcaaacccgtcccggcgtctgtcccacccctcgttccatacgctatcaaggtctttgttatgaagtagactggtctgtccgctctccaacaccacctattagcggttatcctgatattggcacctacacttctggctacatcttcagagactatgactattatagattcaaacctaagttcggtaacggtctatatttaggcaaagtttctgctgctgcttccatcggcacttattctaagtgcggtaaagctcaatccatcagtccataccatgatcacggtattaatactgaccttggcacaccggtttatgatagcgcttgtgactccgcagcttatacaatcccagtagtcaaatataatggaccatactctcttggcgttccagacgttagttgcgaaattcacgatgaaaccttaacatgtggcactaacagcacttttagattttcaatttgttcccataaaatcccttatgacggtccccattcagtcacatgtatcaattctaaagataacaaagtccatattgtgaaacagcccggatattcctactatatcgctggtgacccaggtgcacttcatatctctcacaacaagcataaaccttacacctccattctcaaagatcaaatcaacctcttccacttctcctatctctaccaggcagtagctatgctgtttggaagtctcggttatttcatcttcggtctctatgttactcttttcatcctcaccactctctgggctaacatcaagtatatcttctacgctaaaacaacctaccttggttataccgtgcctcaacgctttatggctggtaagaccactggttgtaagatgtgtggtcttgacactaaacatctcaaggtccatgctcgccatcacaagatctacatccacagtcctatgctcggtcgaactttcttcctctggtctcctatctatgctattctccttctatccattatttctccagcttcagctctcgctccacgacaggcacgtgttagaggctctcctggcaccttcaaacaagaacccgtcatcaaatcaacatcatgctctggtacacaatgcaaagttgaccttgagtatacaggtgttattcccatctatgacggtgctcagtttacagccgatcttaacatcgagggttatcttccaaccacaactagttacatagttcgtgaccccacatacacctcatcctgtgcctatctctacacttctcttcctcctaaactctgcgacgctcgcatcaattggtcttgccttcatactggttcttgcaaaaatagctccgaatatcttttcaagcctttaggtcaacacacctccaacgattacattgttgctaatccatctaatcctcttcaatgcggtctctgtccccattcagctgattgtgatactaaatttgcacatctcaaccacggttgcgctaccatcaacgatggtcacgctgcaggtgccgtttggatttctggcgccgttgacagtgacatgattgctgtcttcgaatgctcagtttccaacatagccttccagatctgtgaccctgccaccaatgaatgcgcatccatcacaacagattatagaaattacaccgcagacttcaagacaattagttttaacatcgctcatccccaagtagctaagacaacatttaaagttggtgcgcttttcacccagagtacaacccatcccaagcatctcctctacgatgttcctggcaagtacgatgctccacctggttctttctttagctatcaagctgagactgttcccaccggtgcattttgtaacaagaacgcatggatgtcccctggcctagcatcaacagacattaaatacaatggcttcccaacattagattataaactaaattttgcaactataaatcaagccattcgcagctacgaccccttaggagctatagttcaatgtaattacgatcagacatatatttccactatcacagcccgtcagcaacgttcacttcttctaaacggagttacttacacagatgaggtagatgctctttcgtatagtctcctcctgaatccgcatcactgcgatttcggagccgtgaacatacacttcgcatattccactactgctcatgtttctctaatagaaggtgaccctgaccaactctctttcgactgtacaggctgtcttttcactaacaaccaaatgcaatgttccatcaaaggtattgactctcactcttatcaaattactgattctcttaatactttcggcgctgcaacatgctctcatactaaagccgatcatctctgtaatttcactgcttccgcatctgagttcgatcttaaagttaatggtaaagcggtcactattactccagttatcacacaatgtgacgtcgggtctatatccgacaccattgttggtgctgcaggtaatgatggctatggtaccttcactcctttcgcattcggcggcaagacatgggattacattctcaagtatattctatatggcatcggcacacctgtctttctcttcgctctaatttgcttgctacagttgctatcccacttatgcacgtccatgagaacgaaagccaaacgatcctag

SEQ ID NO: 19 - DNA, 3815 bp, Decapod penstylhamaparvovirus 1
  source 1..3815: mol_type = genomic DNA
gcttcgcaggaaaccgttacaacatatgacgtcataggtcctatataagagatgacggactcaccggtctcccagtctctaactgacgagtgaagaggctattccaagtgactaaggacaattttggaacatggaagatacgaataaccacccgtggcagtccatacctagaccaacgttatttggatcttcacgtgacagtgaaccaacagaagtctttgaaaacgtattcggagaaagacaagccccaggaaaccaatgtaactactgatgactgactaggtgactatccataacttttctaacagacgcaagtgacgaactttgtcgtcacttacaaaagacgtaaccgctttcgtccgtcactcatataaatctttctctgtctttcagacgacataccccaacaaatatcactgcgctactgtccagatcacattctaccgtggtgcttcatagggaacagacccgttctctactgcctctgcaacgagtgtttcatagacaatctcaatgtcaacggacagtgtcaacactgtcatcccgacgacgaagaatggacagaaaatatggccaaggacatactgcattcacgtcagggcgaaccagaatcacttagtgaattgcttcgagaacgcacgaacgaaactcactccagccagggaatttctccaagccttctcaccccaggtccaaatcaagagcctagacccactaccgaacaacttcttaatatgtctcaagaactgttccagttttcagacgaggaagacaactctcaaactcctccaagaacttcaacaccagaacaaactgatcctaaggtctgcgtggataacctgggaattcgagagggaacaggaaacggaacaattcaacttggaagtgaatcagaaaactcccttggaagtgttggaaacagtgatgacaggggtcaaaagagacagagaggaattacttacatcagtgacacatcagattcctctggatcagatgacgaaaatctggatacaccacatagaaataaaagaaccagaaactccaacaccacaaaagagacaagcggaggaaacggcggaggagatcaggaaagcgatcatggaagcaatggaaatcgactggaacctaccaatggaggagagagcaatagtagcggaacacaacccgactttattgaagggactcccgacggaccggacgaaatggacggaaggcgactggaagagagtgagattgataaacaagtggaaagtgcaacatggtacaccttcgtcatcagagaaaaaccacaaccaagaagactctccggacgcacacaccaaacttcaccattacagatcatggtgaccactggcacatcacatactccggacacccaaccaataagaccagacatagagctacaatcctcgcctatctgggagttacctttgctgccagagccgaagctgaagcgactacggtacttgttagagatatcaagagatggatactctatcttatcagatacggtattgaacggctttcgtattttggtcttggccacgccatttttaaacgaatcatcaaatacttccaacgatacaaaagagacgaagacgcagtagacggaccatgtccatatatgactactacaagagaagacagagctgaagaaaaacctaaagaaaacagtgcagaatatgactacctccaacacttagtcaaaaccaaatctgcaagaacagtccaagaacttgtcaataaacttgacgatgaggaatacaaacagctatggacccgcaccagaggacaatataaagacaaactcaggggaatactaacttactacaacaacaagaaaaagtcaaaccaaagccaactgtcactcattacaaacctgcaaaatatatcaaaaaggaaaccagactacgacaacatgcagtggataaagtacatgttagccaacaacgacatccgtgtaccagaaatcttagcttggataatcatcgtagcagacaaaaaactggacaaaatcaacactcttgtactccaaggaccaacaggaacaggaaaatctcttaccatcggtgcactactgggaaaactgaacacaggactagtaacaagaacaggagactccaacaccttccatctacaaaacctaatcggcaagtcctacgctctcttcgaagaacccagaatcagtcaaataacagtagacgacttcaaactccttttcgaaggatcagacctagaagtaaacataaaacaccaagagtcagaaattatgggacgaatcccaatcttcatatcaacaaacaaggacatagactactgggtaccaccagctgatggtaaagctctacaaacaagaacaaaaaccttccacctaacaagacagataaaaggtctgtcagacaggatgaacagccagtacgacatcaaccctccaccagacaagatcaccagcgacgacttcctaggcctcttccaagaatacgaaaaggaaatcgacgacatcatcgacatcatgtgcgccgattcaacaagatcaagcccaaggtaaaagatccaggagggatgcacataatgaagacgaagaacacgccgagggatcaagtggaccagacccacacagatgtctacaattcaatacgggagactcaatacatattactttccaaacaagaagatacttcgaattcgacgctgccaatgatggaaacttcgacggaaaaaacttatactgcctcccactacattggatgaacttatatctctatggactgaaaagcagtgacagttcagcaacagaaacacagcgatataagatggtaaagtcaatgatgaaaacctacggatggaaagttcacaaagcaggcgtcgtaatgcactcaatggtaccccttatgaaagacttaaaggtatcaggaggaacatcatttgaaactctcacatttacagacaccccatatttagaaatatttaaggatactactggactacataatcaactagcaactaaggaagccgacgtaacattagcaaaatggatacaaaatccgcaacttgtgacagtacaatcaacagcagcaaactatgaagacccaatccaacaatttggattcatggaacaaatgcgaaccggtgacagaaaagcctatacaatccatggtgacactagaaattggtatggcggagaaataccaacaaccggacccaccttcatcccaaaatggggtggtcaattaaaatgggacaaaccatcccttggaaacctagtctacccagcagaccaccatacaaacgactggcaacagatcttcatgagaatgtcaacaatcaaaggaccaaatggagacgaacttaaacttggctgcagagtacaagccgacttcttcctacacctagaagtacgactcccaccacaaggatgtgtagcaagtttggggatgttacaatatcttcacgcaccatgtactggacaacttaacaaatgttatattatgcatactaactaaatatattcgatgtgcaatatatacccacttatatcgcgcttaagccaataaacctatataatctattactatctatacctaccctctacacaaaccagctacccaggcaaggtgggactccggctacccaggcatggtgggacacttctctactattgacgacgtacttcgtcacatacgtcacttacaaaagactaaaatccctatcgtcagtcagtcatttagagtcagggatattgtccgccgtcacttagagcgcgaagcgcgagtatccatcatttaaattagtggtatgacgtcacatattaagta

SEQ ID NO: 20 - DNA, 8226 bp, Decapod penstylhamaparvovirus 1
  source 1..8226: mol_type = genomic DNA
attttctacatctggccaaggaaaatctcttcgtaggcaagaacgaagtggctatggggtttgaggtttagccgaaagagatctcgacaatctttagtaggatacacttcttacactcctggaaaagaaaaacattatctgtccagcaatgatacacgggtacgtcctccattagctgttgttttcttttcctcggcaggtcctacagtgtcggagcgcaggaacaggacgaacccacgagtcgcatccttacgagaagtgcagtaggttggcgctgttgagagcgggtgatctgggtaatgtccgtcattacgggtagcattacttggcgcatgcagtgggaagtcatggcccacgtcaccttaacaaaagactagtcccactgtaacctcggcgtatagcgaaagcggttagataacattaaataccggcttgtatgctagaaactggactgtcaatataccattgaatgtctcaaacaaagaataaagaacctgttgttgaactgcaaaatcaacaaaccttcaaccaaaaagaccacaatgcgctagtgattgaacacaaaactagtgcaacaacgagcgcccaaggcattcccgttcgtccttcacgtaagaaggatgattatgtggacatgacacagggcaatttcaacctaattctaaaactgttaccaacaatttctgaactacagaagaggcatattcttcatctacttcgagaagaagtagaaggaaagaaagtttgtttcgtagagcgagaaaaacagaatccacttatggctataaacgaactagccgttaaagttggaaagaagcctaaatacacatcgacgaaaaccggagctgaccacattccaagctggactgtattggtcgagttcgcaggttttagcgaagcagcaacatgtgacacagttaaaaacgcaaaaatgattgctgcttacaaattagttaaaagattttgtaaatgggacccaacctacattgaaatttctgattgtatgctgccacctccagaccttacatcgtgcggggacgttgagagtaatcctggacctatcatacatagcgttgcatttgcaagaactggttcagtatggacacctgccaactttactttcaataccacatcatccccgggtagactgcaagtacaaatgtcatccagcgacaatagatatgggctcaattctgttttacttgcagcaggtggtacaacatggggcacagcttatttttcacaacgctggaacagtgactttggattacagccagatctaactataactgtaacaccacaactaactggagaagaacttggttcaatttattttgatgctgttgaaactacaaatgcagaggaagctatagaggcaacaattataaatactactccaattgatgtaatggtagacacaacagcaggccctgtaccaattgaaggtgatttcaaacccacaaatgaaactcccacgtgggtaagtaacacaaagcccattgatgtagcgacacataagactcgtgactacgcaaatcggtcagtactgactccacaattgatcacaaaaatgaaggaccatgtctatgccttacaaaaggatgaagttaaagatgtaacacttgcctcactcgatttcggcttaaatccatcaagtgtactcggaagatggttgaaatggttaactggcatagacatgagactccatcttggaaagaaagtcacaaaccataaagttagaatgtttgtgcaaaaaacaaggtttccactaaagacagcaattgaggaacttaacaatggtactattccacatagattaaggagggatgttcgttacattgaaaagccgttcgataaagaggaacatactgacatactgttatctggtgacattgaggaaaaccctgggcctgaagggatggaacaggtatcaataacaccagaacaacttcaaatgattctggcaatgcaaaaatcaccaccaaagccaaagatcaaagaactaagtgaaagggaaattcagttaaggctccaaggaaatgaagcaacccgcagacatcaaagatttaaaggtgagcccattgatccagttctaactagagaagatataattagaaagcacaatcagcagaatggaatattgcctgatgaaaaagaacagccggtagtggtaaataacgtacaccctttggagaaggagtattccattgaaaaggaaggaatggtgtgggacgaagaacaatttttgacgtatatacatgacaagtcatcaagcaacgatcactatgcctgcatctataacgttattacacataacagattcggtgtacttgaaatgccagaagatccacttgaaatgattgatcattatgagccagaggaagaaccaaaaacaaaacctaaaactaaaagtggtaacaagagagaactaaatgatgaaaccttttacagaaagaagaagacaaaaacaacaaaagagccaaaaacacaagaacaaaagaaaattgaccacgataacatgttttcacgattgctgagaaggcttgacaaaccacaaattgtagcagtttggctaaacaacagaccatcacgtaaattggtcgaaaaattagctgagagtaaattcggaattggatggcaagcaaaagaagagtacacaaccagcatggtaattgtatctggatatatcaattgtgaaccattacccctaatcgtagataagcttctctccgtcgataacaactatgacatgtggcaacagactgacaagtattttaacaatttaatcacactgtgtaaccgcgtcagtgacgttacctacactagcgctcagctgtgtagagatgcatctatcttgaataacaagaagatgcatgttgaaaatggaaacattgtttcaatggaaaatcagtctgaaattgatagtcaaactaaattcttttcactgttagaagatgataacaaacttccaattgtagatgagctaagagtattagctgatatgacggcacaaagaagcaacgtaaacactgctggtaatcatcttcgtgataatgactctattagggccgacgctgttctagctaacaatacagtacgtaacaattgtcaaattccaattcctgtaacaacactaataccaagacaaattcgtggattgaatggtgtacttgtaaatcagcaattacggttacagggaattgaaacacacattacagacagttatatttcaaaagctgagccatctgactattcgaaacaactgtctgaaatggttaatgctcaaaaaacatcaacttggcgagcaaacaatatcgcatcacaggggtgggacatgtttgatactgtacagttaaatacaaacatatcgcaaaaagatctttcaatggacactgctttgacaaagcttatgttgttgtaccagctaacaacacaaaatctgccagcaacacaattaccatcaagcatttattctgcatttgattcaagaacacagcccactttacaggatggaatttggggtataaataatggtgctaatatatttggtgaacaatgcggtggattagccgcgccagtctttccatttagtgggggcaccggagaaattactttccatcttactttacaatctgttccacaggaatttcaagaatcagcaattttcgtaccagcaactgcactacaagctgcaaaagagggtgctcgaacattggcaatgtatgttttaatgtttgcagaatggccatttggtatgtatacaaaaactaaacaaacaacagacaatgctggtaataatcaatcagatcaaattttcattcactccgaatctactgtacatattccaggacaaaaacaaatgcatattgtgctgccaagaaaagtgaacatggtgaaccccactacaattgcagaagcaaatgcacgtgtagtaattcaaccaacatacggtacagtggcagctggagcaggtgtcgcaaatggtaatattaacgtagctgctattggtgtggccttgccaactgtaaatttgactgactatcttgtatcctgggcaaccgatttcacacttggcgacataaaacaattgattgaaagaatgaaaacaacactgccaattagtcgagacttgatggcagcacgtcaaaatgctatgttattgagtactctatttcctccactaattcagagcaatgtggcttcagacacaaaggaagtcccaggaacagctggagcatacactgcatgtcttgcaaacttaggtattcctgaaacactaacagttaactggggagaagatataaatgttcagccattgtatcagctacttgaaacggacatcacagcccacaatcggtacgtattaaacctgtttaaaagagaagaagtggtagctggtgcatatgaatttggatggttaggacacatggccagttatatgatgggactccttctaacaatgaatatatcatcagtgtttaacgtctggtattcaacaagacgtatttcaacaaaggcgtgggacacagcatatgatagtaacatccaagcatatcaggacatgcattatcaaatgttttcgtggagttcaatgcaaggtagtattgcaccagcaatggtggacgaaattcttcataacctttgtggccaaatgtttgggttcagcttaccattgagacaagtcttatttaacgcattgccaatcactttttcatcgtttggaagttggatgtcgcctagagtttctgatggtttccaaactgtaaggtattatgatataggtccaccagtcattaatgcaaaacgtgatggggaagtaccagtaagtatgattgacgcatggacttataaatttacagaaaaattgcccaaaagttttttgccatggccaatgccagaaggaaaggacagtacaatgggatatgatccggaaaaagaaccagcactaattgataattcaaatgagacaggcaatgtattcagaccatttatggcaagaaatggcaacaattctaattatttgccaaccaactacacaattgacgtatcacagaatggtcatgacgagagttgtattaatgttgacctttttaacaatgttgcgggagtaacactaacaaattatgatggaaccgcaacaaacgcagacgtcgtaccaacaggatcatacattaagcagagagcaatgcctatcgatgcaaatgcggtacgaccaactgaaacactcgacgctgctaaccatacaaaaccttttgctattgaaggaggaagactcgtatatttgggtggaacaattgcaaatacaaccaatgtggtaaacgcaatgcagaggaaacaaaggctttcaaaaccagcattcaagtgggcacatgctcagagacaacgtgtatatgacagcagtcgtccagggatggacccaatcacaaagttgtgtgcacgaaagtcgggttttatgaatgcccgttccacagcaatgatggcacccaagactggactcagcgctgttatagatcaagcaccaaatacatctcaagacttgatcgaacaaccgagtcagcaagaggttatggatatgcaagcgacagcaacagtataaatcagatatatcaaattgcattgcatagaaaggcaaaacatataacagcaaagaaatggcaagaattaacaaaaggtatttataatgcatctaccctgacaccgaagatagttgaccaaattataaaggatgaaggaagcgggaccgataagacaaaatatgtaaatgttcctaaaataattactgacaaagaattacaaacattctatgtaccaagaagcaacgcagatctagttataagaagaatacgtttaatcgacctttggcgtaacctaaaaccagatcaaatggacgagattcgtaattacactcatctagattatatctttgtacaaaacatttgtatctatatgttagtatttggaatagacacagttaaacactttagacaaataggtctattcaatgaaaggaatgaatttattgaaattgcaaaacagttatcaaccaaagggaaaagatttgttgatgatgttgataatatgaaacaaaaggtatgtgaaatagctactattgttggttatatggacccaaatgttgataaaatagatgtaatggaagaagtcaactcactcgcagctgaaggtaatgaacatggtatagatagagataattggaatgatttgtttacaaaaacatgcaaagaagtaatgacatggtacaaaggacatgaatttattagttttgatgattacataaaggaaggaatgtggttaacaagtggaagttcaagtattgggaaagtggattggacaaaagatggagaaaatgggaaatttaaagcaaggaaaaatatgttgttacaaatttatacaccgcaagaattggccaacattgtttatgcttgggatggaaagttacattcacgtgtctttattaaaaacgaaatgagtaaattaagacttgctgtggcatctaacatcgaagcatatattcatgaatcttatatgcttttcctatatggtcatggttttaaagaatactttggagtgacgcttgacgaaaaaccagatcaacagcatcagagagaaattgaaatgattgagaaactacaagctggatactttggattaccatttgactatgcatcatttgatcatcagccaacaactttcgaagttaagacaatggtgagaagagttggagaaattgtagttagtcaagtacctaagaattattactatcagacacaaatgctagtcaacaagattgttaatgcatatgataaaagttatctgtctggaaatattaaaaatacaaaatttgaaaatataaaagtcaaaggtggagtaccatcaggagtgagaataacgagtttgttgggtaacatgtggaacgctataattacaaagatcgcaataaataatgttattggaattattggatacgacccaatctcccaaatctcgttacgcggagacgacgttgctatattaagtaaagatccagcagctctttatttacttagactatcatatgctgcaattaatgcaattggcaaagatagtaagttaggtatatctccaaaagtatgtgaatttttacggaatgagatatcagtgacaggagtacgcggttggacatgtagaggaataggtggcataagtcaacgaaaaccatggaatccacaaccttggagtccaaatgatgaagtcgaaacaaatgcaagcaacatttcattattagaaagacgagcaggaattgaacttcaacaattacaccacataaacaaagttaaatggtcgagacacgtcagacaaagttataaatatctagaattgccaaaacgacttggtggttttggaatctatcgatttcaggggtggttacctaacggcaaattaccacttgccaaaaagcccttggttaacgtagaggacatacatccaagccaagagctgtttttgcctttaagtgaacaacagaaaaagatcttagcacaggttgaaatgacaaacaaaatgcaaacagatgatatacctgggacacaaaaattattttcaaaggaatggatacagaaagtgcgtgcaaaaaagatcatatggagtagaaatcagacaataccaatccatactgatcacacagtacgtataccaagatgggacgagaaaatcaaattcccaaggtataaatcacaatatatattaaataataaaatcaacttaacaatggagcaagtgttaagacagtataatctcctaaaagaagtggaacggtatgataaagatctaaaagtgccaaagttattagacattttggacaaatggttcccggtccaaagtagtaaaattaaaacatatgaaagtcaaggttttcatagaacagacgcaattaatcttgctgttggtgaaattccaactgaaccagcagttaaaataaatcctatactaataaactttgttaagttacatcttgaaagacaaggtattagacatcagagaggtagaaataagatcgccaaatttatttaccaaaaaacaaaacaagcagagaacatgatactgcaaagtagtttacaacaaatgtataggtactaaattaagggaccaataaagaaacttcgagtttcctataacacattcccagttgggttttgtggccagccatgcggttggccctaggttatagtc

SEQ ID NO: 21 - DNA, 2731 bp, Infectious pancreatic necrosis virus
  source 1..2731: mol_type = genomic DNA
acccgacataccacgactgtttatgtatgcacgcaagtgccccctaacaaatctctatacacacaactcatgatatgtcggacatcttcaactcaccacagaacaaggcttctatcttgagcgctctcatgaagagcacaacaggagatgtagaagacgtgctgatacccaagcgcttcaggccagccaaggatccccttgacagcccgcaagccgcagcacagttcctaaaggacaacaagtatcggatacttaggccgcgagccattccgaccatggtcgaactagagacagatgccgctctgcctcgactgcgacaaatggtagacgatggcaagcttaaggacacggtaagcgtcccagagggaaccactgcattttacccaaaatactacccattccacaagccagatcatgacgaagttgggacgttcggggctccggacatcacacttctgaagcaactcaccttcttcctgttggaaaatgacttccccacaggaccggagacactcaggcaagtccgcgaggccatagccacactccagtatggctcaggcagctactccggacaactgaacaggctcctagcaatgaaaggagttgctactggcaggaatccaaacaagactccaaaaacagtaggctacaccaacgagcagctggccaaactgctggagcaaaccctaccgatcaacactccaaaacatgaggaccccgacctccggtgggcccccagctggttgatcaactacaccggagatctgagcacagacaagtcatacctgccacatgtgaccataaagtcctcagccggcctaccgtacataggcaaaaccaaaggagacacgactgccgaagcgctcgtactggccgactccttcatacgtgaccttggaaaagcagcaacgtcagcggacccagaagctggggtgaagaagacaatcaccgacttctggtatctaagctgtgggctgctattcccaaagggcgagagatacacacagatcgactgggacaagaagaccagaaacatctggagtgcgccctacccaacacacctactactatcaatggtgtcgtcccctgtgatggacgagtcaaaactcaacatcaccaacacccagaccccatctctgtacgggttctcaccattccacggagggatggacaggatcatggcaatcatcagagacagcctggacaacgacgaggacctagtgatgatatatgcagacaacatctacatcctgcaagacaacacgtggtactcgatagacctggagaagggcgaggccaactgcaccccacaacacatgcaggccatgatgtactacctcctgacaagggggtggacgaacgaggacggctctccacggtacaacccgacgtgggcaaccttcgccatgaacgttgcaccgtcgatggtcgtggactcatcctgcctgctaatgaatcttcagctgaagacctacggccagggcagtgggaacgcctttaccttcctgaacaatcacctcatgtccacgattgtcgtggctgagtgggtaaaagcaggaaaaccaaaccccatgacaaaagggttcatggacctcgaggaaaagacggggattaacttcaagattgagcgagagctaaagaaccttaaggagaccatcatcgaggccgtggaaacggctccccaggatggctacctgcgcgatggctccgacctaccaccgcacaggccaggaaaagcagtagagcttgacctgctgggctggtcggccatctacagccgccaaatggagatgttcgtcccagtcctcgagaacgaacgactaattgcatcagccgcccacccaaaagggcttgagaacaagacccttgcccggaaacccggggccgagattgcatatcaaatagtaaggtacgaagcaatcaggatggtgggcggctggaacaatccactgctggaaaccgcagcaaaacacatgtccctcgacaagaggaagagactggaggtgaaggggatcgacgtgacagggttcctcgacgactggaacaacatgtcagagttcggaggagacctagaaggaatcacactatccgaacccctcacaaaccagaccctgatagatattaacacaccactggagagcttcgacccaaaggcgagaccacaaacacccaggtctccaaagaaaaccctagacgaggtaacagccgccataacatcggggacctataaggaccccaagagcgcagtgtggcgactgcttgaccaaagaaccaaactccgggtcagcacactgcgagatcaggcctcggcgctgaaaccggcatcatcctcagtagacaactgggccgaagccacggaggaactagcggagcagcaacaacttctcatgaaggccaacaacctgctgaagagcagcctgacggagaccagagaagcactggaaaccatccagtccgacaaaatcatcgctggaaaatccaatcccgaaaagaacccgggaacagcagccaacccagtggttggctatggggaattcagcgaaaagattcctctgactcccacgcaaaagaagaatgccaagcggagggagaagcagagacgaaaccagtgagaagaacaaaccggggagaatccgaaatgacccagctggactcatatgcaagctccgcgccgcacggcaagctgaacaaaagtagtgacccgacaacgtgccaccaacatgaccccaga

SEQ ID NO: 22 - DNA, 1180 bp, Infectious pancreatic necrosis virus
  source 1..1180: mol_type = genomic DNA
tgagatccattatgcttccagagactggaccagcaagcatcccggacgacataacggagagacacatcttaaaacaagagacctcgtcatacaacttagaggtctccgactcaggaagtggcattcttgtttgtttccctggagcaccaggatcaagggtcggtgctcactacagatggaatgcgaaccagacggagctggagttcgaccagtggctggagacgtcgcaggacctgaagaaagccttcaactatggaaggttgatctcaaggaaatacgacatccaaagctcaacgctgccggccggcctctatgcactgaacgggacgctcaacgctgctacattcgaaggcagtctatctgaggttgagagcctgacctacaacagcctgatgtctctaacaacgaacccccaggacaaagtcaacaaccaactggtgacaaaaggagtcacagtcctgaacctaccaacagggttcgacaaaccatacgtccgcctagaggacgagacaccccagggtcttcagtcaatgaacggggcaaagatgaggtgcacagctgcaattgcaccgcggaggtatgagatcgacctcccatcccaacgcctaccacccgtcccagcgactggaaccctcaccacaatctacgaggggaacgcagacatcgtcaactccacaacagtgacgggagacatcaacttcagtctggcagacaatccccccgcagacatcaggttcgacttccagctggaattcctgggccttgacaatgacaccccagtggtcaccgtggtcagctccgtgctggcaacacccgacaactatcgaggcgtgtcagccaagatgacccagtccataccgaccgagaacatcaccaaaccggtcacgagagtcaagctgtcatacaaagtcaaccaacaggctgccatcggcaacgtcgccaccctgggcgcactggggcccgcgtccgtgtccttctcatcaggaaacggaaacgtgcctggcgtgctcagaccaatcacactggtggcctatgagaaaatgacaccactgtccatcctgaccgtagcaggagtgtccaactacgagctgatcccaaacccagaactcctgaagaacatggtgacacgctatggcaaatatgacccagaaggtctcaactatgccaagatgatcctgtc

SEQ ID NO: 23 - DNA, 852 bp, Infectious pancreatic necrosis virus
  source 1..852: mol_type = genomic DNA
agaggaattcgaaaagtcgcagctcctgtactgtcaacgctgtttccaatggcagcaccactcataggaatggcagaccaattcattggagatctcaccaagaccaacgcagcaggcggaaggtaccactccatggccgcaggagggcgctacaaagacgtgctcgagtcctgggcaagcggagggcccgagggaaaattctcccgagccctcaagagcaggctggagtccgccaactacgaggaagtcgagcttccgcccccctcaaaaggagtcatcgtccctgtggtgcacacagccaagagtgcaccaggcgaggcattcgggtccctggcaattataattccaggggagtaccccgagcttctagatgccaaccagcaggtcctatcccacttcgcaaacgacaccgggagcgtgtggggcataggagaggacatacccttcgagggagacaacatgtgctacactgcactcccactcaaggagatcaagagaaacgggaacatagtagtcgagaagatctttgctgggccaattatgggtccctccgctcaactaggactgtccctacttgtgaacgacatcgaggacggagttccaaggatggtgttcaccggcgagatcgccgacgatgaggagacaatcataccaatccgcggtgtagacatcaaagccatcgcagcccacgaacaagggctgccactcatcggcaaccaaccaggagtggacgaggaggtgcgaaacacatccctggccgcacacctgatccaggccgggaccctgcccgtacaacgcgcacagggctccaacaagaggatcaagtacctgggagagctgatggcatcaaat

SEQ ID NO: 24 - DNA, 1734 bp, Infectious pancreatic necrosis virus
  source 1..1734: mol_type = genomic DNA
ggaaagagagtttcaacgttagtggtaacccacgggcggagagctcttacggaggagctctccgtcgatggcgaaagccctttctaacaaacaaacacaattaatatcaatgcaagatgagcacaaacaaagcaaccgcaacctacttgaagtccattatgcttccagagactggaccttccagcattccggacgacataacggagagacacatcttaaaacaagagacttcgtcctacaacttagaggtctcggactcaggaagtggtgttcttgtttgtttccctggagcaccaggatcaagggtaggtgctcactacagatggaatgtgaaccagacggaactagagttcgaccagtggctggagacgtcacaggacctgaagaaagctttcaactacgggaggctgatatccaggaaatacgacgttcaaagctccacgctgccggctgggctctatgccctgaatgggaccatcaacgccgccaccttcgaaggcagtctgtctgaggttgagagcctgtcctacaacagcctgatgtctctgacaacgaacccccaggacaaagtcaacaaccagctcgtgaccaaaggagtcacggtcctgaacctgccaacagggttcgacaaaccatacgtccgactagaggacgagacaccccagggtctccagtcaatgaacggggccaagatgaggtgcacagctgcaaccgcaccggggaggtacgagatcgacctcccatcccaaagactgccaaccgtccctgcgactggaaccctcaccacgatctacgaggggaatgccgacatcgtcaactcgacgacagtcactggagacattagcttcagcctcgcaaacaaccccaccgcagacatcaagttcgacttccagctggacttcctcggtctcgacaacgacgtcccggttgtcacggtgaccagctccgtgctggtaaacgcagacaactacagaggcgcgtcagccaagatgacgatgtccatacccaccgagaacatcacgaagccgatcacaagagtcaagctgtcctacaaagtcaaccagcagacagcgatagccaacccagccaccctggggacactaggtccagggtccgtctccttttcttcaggaaacggcaatgtccccggtgtcctgagacccatcacactggtggcctatgagaaaatgacaccccagtccatcctaactgtagctggagtgtccaactacgagctgatccccaacccagaactcttgaagaacatggtgacacgctatggcaagtatgaccccgaagggctcaactatgccaagatgatcctgtcccacagggaggagctggacataaggacagtctggaagactgaggagtacaaggagcggacaagagtcttcaacgagatcaccgacttctccagtgacctgcccacgtcaaaggcatggggctggagggacatagtcagagggatccggaaagtcgccgccccagtactgtcaacgctgtttccgatggcagcaccactcattggagtggcagaccaactcatcggagatctcaccaacaccaacgcagcaggcggaaggtaccgctccatggccgcaggaggacgctacaaggatgtaatggactcctgggccagcggcggacccgacgggaagttctcccaggctctaaagaacagactggagtctgccaactacgaggaagtcgagcttcctcccccttcaaaag

SEQ ID NO: 25 - DNA, 767 bp, Litopenaeus vannamei
  source 1..767: mol_type = genomic DNA
ggactgcgcagtgacatcgccatattgactggaagcgccgtgctgstggctcggatttctcccgcttatttccttcccaaaatacatcatggcatctcgcaagaagattctcctgaaggtgatcmtccttggagactcaggtgtaggcaaaacatcccttatgaaccagtttgttaacaagaaattcagcaaccagtataaggcaaccattggagcagatttcctcacaaaggaggtcatggttgatgacagattggttaccatgcagatctgggatacagctggtcaagaaagatttcaatcgttaggtgttgcattctatcgaggagctgattgttgtgttctcgtctatgatgttacatctcccaataccttcaagtctctcgattcatggcgtgacgagtttctaattcaagcctcaccaagggaccctgaccacttcccatttgttgtcctgrgtaacaagattgatctggagaatagggcggtatcgacgaagcgagcacaacaatggtgtcatagtaaaaatgaarttccctactttgaaactagtgcaaaggaagctattaatgtggagctagctttccagaccattgcycgcaatgctcttgctcaggagtcagaggtggagctgtacaatgagtttccagaccagatcaaattgaccaatgacaacaaggctaaacaggatgcgtgctcttgctaatgatcactctgtaatgattttctagtacggaaaaaaaaaaaaaaaaaaaaaaaaaaaaaaa

SEQ ID NO: 26 - DNA, 34 bp, synthetic construct
  misc_feature 1..34: note = Single strand DNA oligonucleotide
  source 1..34: mol_type = other DNA
gaaactctagatgggtaacaagattgatctggag

SEQ ID NO: 27 - DNA, 20 bp, synthetic construct
  misc_feature 1..20: note = Single strand DNA oligonucleotide
  source 1..20: mol_type = other DNA
agccggatcctagcttacga

SEQ ID NO: 28 - DNA, 34 bp, synthetic construct
  misc_feature 1..34: note = Single strand DNA oligonucleotide
  source 1..34: mol_type = other DNA
gcataggatcctgggtaacaagattgatctggag

SEQ ID NO: 29 - DNA, 34 bp, synthetic construct
  misc_feature 1..34: note = Single strand DNA oligonucleotide
  source 1..34: mol_type = other DNA
gagtactgcagcatcctgtttagccttgttgtca

SEQ ID NO: 30 - DNA, 31 bp, synthetic construct
  misc_feature 1..31: note = Single strand DNA oligonucleotide
  source 1..31: mol_type = other DNA
gatctctagagaggtggagctgtacaatgag

SEQ ID NO: 31 - DNA, 19 bp, synthetic construct
  misc_feature 1..19: note = Single strand DNA oligonucleotide
  source 1..19: mol_type = other DNA
cgtctgcagcgcatcttgc

SEQ ID NO: 32 - DNA, 31 bp, synthetic construct
  misc_feature 1..31: note = Single strand DNA oligonucleotide
  source 1..31: mol_type = other DNA
gatcggatccgaggtggagctgtacaatgag

SEQ ID NO: 33 - DNA, 33 bp, synthetic construct
  misc_feature 1..33: note = Single strand DNA oligonucleotide
  source 1..33: mol_type = other DNA
gactactgcagcatcctgtttagccttgttgtc

SEQ ID NO: 34 - DNA, 33 bp, synthetic construct
  misc_feature 1..33: note = Single strand DNA oligonucleotide
  source 1..33: mol_type = other DNA
gaatcggatcctaatgtggagctagctttccag

SEQ ID NO: 35 - DNA, 34 bp, synthetic construct; identical to SEQ ID NO: 29
  misc_feature 1..34: note = Single strand DNA oligonucleotide
  source 1..34: mol_type = other DNA

SEQ ID NO: 36 - DNA, 20 bp, synthetic construct
  misc_feature 1..20: note = Single strand DNA oligonucleotide
  source 1..20: mol_type = other DNA
tacgtctgcagcgcatcttg

SEQ ID NO: 37 - DNA, 33 bp, synthetic construct
  misc_feature 1..33: note = Single strand DNA oligonucleotide
  source 1..33: mol_type = other DNA
gaatctctagataatgtggagctagctttccag

SEQ ID NO: 38 - DNA, 21 bp, synthetic construct
  misc_feature 1..21: note = Single strand DNA oligonucleotide
  source 1..21: mol_type = other DNA
gggatacagctggtcaagaaa

SEQ ID NO: 39 - DNA, 22 bp, synthetic construct
  misc_feature 1..22: note = Single strand DNA oligonucleotide
  source 1..22: mol_type = other DNA
cgagagacttgaaggtattggg

SEQ ID NO: 40 - DNA, 26 bp, synthetic construct
  misc_feature 1..26: note = Single strand DNA oligonucleotide
  misc_feature 1: note = 5' 6-FAM
  source 1..26: mol_type = other DNA
cgaggagctgattgttgtgttctcgt

SEQ ID NO: 41 - DNA, 22 bp, synthetic construct
  misc_feature 1..22: note = Single strand DNA oligonucleotide
  source 1..22: mol_type = other DNA
gtggagaccttccaacagtatg

SEQ ID NO: 42 - DNA, 22 bp, synthetic construct
  misc_feature 1..22: note = Single strand DNA oligonucleotide
  source 1..22: mol_type = other DNA
ccttcttgttgacctccttgat

SEQ ID NO: 43 - DNA, 21 bp, synthetic construct
  misc_feature 1..21: note = Single strand DNA oligonucleotide
  misc_feature 1: note = 5' 6-FAM
  source 1..21: mol_type = other DNA
tgcgtgacatgaagcagacgg

SEQ ID NO: 44-45 - skipped (2 entries)

SEQ ID NO: 46 - DNA, 614 bp, synthetic construct
  misc_feature 1..614: note = Rab7-fragment plus non-coding addition for the loop used as template for amplification
  source 1..614: mol_type = other DNA
ggcaatagcatgccgtcctctagattacatctcccaataccttcaagtctctcgattcatggcgtgacgagtttctaattcaagcctcaccaagggaccctgaccacttcccatttgttgtcctgggtaacaagattgatctggagaatagggcggtatcgacgaagcgagcacaacaatggtgtcatagtaaaaatgaagttccctactttgaaactagtgcaaaggaagctattaatgtggagctagctttccagaccattgctcgcaatgctcttgctcaggagtcagaggtggagctgtacaatgagtttccagaccagatcaaattgaccaatgacaacaaggctaaacaggatgccatggcccaaagacttaagagtctatcactcctaggcacctttcccggatataaacgccaggttgaatccgcattaggagctacgatggatgagtctgggtggagcgcgctcgatttataccgtgagtaggctcgtgcaagaaccgcaagatgcgctgcagacgtaaccgtaatcgtaagctaggatccacgctagcataaccccttggggcctctaaacgggtcttgaggggttttttggcatatgaaagca

SEQ ID NO: 47 - DNA, 336 bp, synthetic construct
  misc_feature 1..336: note = Rab7 coding sequence
  source 1..336: mol_type = other DNA
ttacatctcccaataccttcaagtctctcgattcatggcgtgacgagtttctaattcaagcctcaccaagggaccctgaccacttcccatttgttgtcctgggtaacaagattgatctggagaatagggcggtatcgacgaagcgagcacaacaatggtgtcatagtaaaaatgaagttccctactttgaaactagtgcaaaggaagctattaatgtggagctagctttccagaccattgctcgcaatgctcttgctcaggagtcagaggtggagctgtacaatgagtttccagaccagatcaaattgaccaatgacaacaaggctaaacaggatg

SEQ ID NO: 48 - DNA, 162 bp, synthetic construct
  misc_feature 1..162: note = Loop noncoding sequence
  source 1..162: mol_type = other DNA
ccatggcccaaagacttaagagtctatcactcctaggcacctttcccggatataaacgccaggttgaatccgcattaggagctacgatggatgagtctgggtggagcgcgctcgatttataccgtgagtaggctcgtgcaagaaccgcaagatgcgctgcag

SEQ ID NO: 49 - DNA, 48 bp, synthetic construct
  misc_feature 1..48: note = T7 terminator
  source 1..48: mol_type = other DNA
ctagcataaccccttggggcctctaaacgggtcttgaggggttttttg

SEQ ID NO: 50 - DNA, 22 bp, synthetic construct
  misc_feature 1..22: note = Single strand DNA oligonucleotide
  source 1..22: mol_type = other DNA
catcctgtttagccttgttgtc

SEQ ID NO: 51 - DNA, 23 bp, synthetic construct
  misc_feature 1..23: note = Single strand DNA oligonucleotide
  source 1..23: mol_type = other DNA
tgggtaacaagattgatctggag

SEQ ID NO: 52 - DNA, 47 bp, synthetic construct
  misc_feature 1..47: note = Single strand DNA oligonucleotide
  source 1..47: mol_type = other DNA
ggatcctaatacgactcactataggcatcctgtttagccttgttgtc

SEQ ID NO: 53 - DNA, 237 bp, synthetic construct
  misc_feature 1..237: note = Single strand DNA oligonucleotide
  source 1..237: mol_type = other DNA
tgggtaacaagattgatctggagaatagggcggtatcgacgaagcgagcacaacaatggtgtcatagtaaaaatgaagttccctactttgaaactagtgcaaaggaagctattaatgtggagctagctttccagaccattgctcgcaatgctcttgctcaggagtcagaggtggagctgtacaatgagtttccagaccagatcaaattgaccaatgacaacaaggctaaacaggatg

SEQ ID NO: 54 - DNA, 237 bp, synthetic construct
  misc_feature 1..237: note = Single strand DNA oligonucleotide
  source 1..237: mol_type = other DNA
catcctgtttagccttgttgtcattggtcaatttgatctggtctggaaactcattgtacagctccacctctgactcctgagcaagagcattgcgagcaatggtctggaaagctagctccacattaatagcttcctttgcactagtttcaaagtagggaacttcatttttactatgacaccattgttgtgctcgcttcgtcgataccgccctattctccagatcaatcttgttaccca

SEQ ID NO: 55 - DNA, 595 bp, synthetic construct
  misc_feature 1..595: note = 250 VP28 RNAi fragment- synthetic template
  source 1..595: mol_type = other DNA
ccatgggcagcagccatcatcatcatcatcacggatccggcaggtatcacaacactgtgaccaagaccatcgaaacccacacagacaatatcgagacaaacatggatgaaaacctccgcattcctgtgactgctgaggttggatcaggctacttcaagatgactgatgtgtcctttgacagcgacaccttgggcaaaatcaagatccgcaatggaaagtctgatgcacagatgaaggaagaagatgcggatcttgtcatcactcccgtggagggccgagcactcgaagtgactgtggggcagaatctcacctttgagggaacattcaaggtgtggaacaacacatcaagaaagatcaacatcactggtatgcagatggtgccaaagattaacccatcaaaggcctttgtcggtagctccaacacctcctccttcacccccgtctctattgatgaggatgaagttggcacctttgtgtgtggtaccacctttggcgcaccaattgcagctaccgccggtggaaatcttttcgacatgtacgtgcacgtcacctactctggcactgagaccgagtaagcggccgcaactcgagaacg

SEQ ID NO: 56 - DNA, 33 bp, synthetic construct
  misc_feature 1..33: note = Single strand DNA oligonucleotide
  source 1..33: mol_type = other DNA
gactatctagacattcaaggtgtggaacaacac

SEQ ID NO: 57 - DNA, 31 bp, synthetic construct
  misc_feature 1..31: note = Single strand DNA oligonucleotide
  source 1..31: mol_type = other DNA
acgtaccatggctcggtctcagtgccagagt

SEQ ID NO: 58 - DNA, 31 bp, synthetic construct
  misc_feature 1..31: note = Single strand DNA oligonucleotide
  source 1..31: mol_type = other DNA
acagactgcagctcggtctcagtgccagagt

SEQ ID NO: 59 - DNA, 250 bp, synthetic construct
  misc_feature 1..250: note = VP28 PCR product
  source 1..250: mol_type = other DNA
attcaaggtgtggaacaacacatcaagaaagatcaacatcactggtatgcagatggtgccaaagattaacccatcaaaggcctttgtcggtagctccaacacctcctccttcacccccgtctctattgatgaggatgaagttggcacctttgtgtgtggtaccacctttggcgcaccaattgcagctaccgccggtggaaatcttttcgacatgtacgtgcacgtcacctactctggcactgagaccgag

SEQ ID NO: 60 - DNA, 46 bp, synthetic construct
  misc_feature 1..46: note = Single strand DNA oligonucleotide
  source 1..46: mol_type = other DNA
ggatcctaatacgactcactataggggcagaatctcacctttgagg

SEQ ID NO: 61 - DNA, 20 bp, synthetic construct
  misc_feature 1..20: note = Single strand DNA oligonucleotide
  source 1..20: mol_type = other DNA
gttggagctaccgacaaagg

SEQ ID NO: 62 - DNA, 21 bp, synthetic construct
  misc_feature 1..21: note = Single strand DNA oligonucleotide
  source 1..21: mol_type = other DNA
ggcagaatctcacctttgagg

SEQ ID NO: 63 - DNA, 45 bp, synthetic construct
  misc_feature 1..45: note = Single strand DNA oligonucleotide
  source 1..45: mol_type = other DNA
ggatcctaatacgactcactatagggttggagctaccgacaaagg

SEQ ID NO: 64 - DNA, 125 bp, synthetic construct
  misc_feature 1..125: note = Single strand DNA oligonucleotide
  source 1..125: mol_type = other DNA
ggcagaatctcacctttgagggaacattcaaggtgtggaacaacacatcaagaaagatcaacatcactggtatgcagatggtgccaaagattaacccatcaaaggcctttgtcggtagctccaac

SEQ ID NO: 65 - DNA, 125 bp, synthetic construct
  misc_feature 1..125: note = Single strand DNA oligonucleotide
  source 1..125: mol_type = other DNA
gttggagctaccgacaaaggcctttgatgggttaatctttggcaccatctgcataccagtgatgttgatctttcttgatgtgttgttccacaccttgaatgttccctcaaaggtgagattctgcc

SEQ ID NO: 66 - DNA, 925 bp, synthetic construct
  misc_feature 1..925: note = Synthetic DNA template for production of a mix of four WSSV gene fragments VP28, VP19, rr2 and wsv477
  source 1..925: mol_type = other DNA
gacagatatggccaccacgactaacactcttcctttcggcaggaccggagcccaggccgctggtccttcttacaccatggaagatcttgaaggctccatgtctatggctcgcatgggtctctttttgatcgttgctatctcaattggtatcctcgtcctggccgtcatgaatgtatggatgggaccaaagaaggacagcgattctgacactgataaggacaccgatgatgatgacgacactgccaacgataacgatgatgaggacaaatataagaacaggaccagggatatgatgcttctggctgggtccgctcttctgttcctcgtttccgccgccaccgtttttatgtcttaccccaagaggaggcagtaaggcagaatctcacctttgagggaacattcaaggtgtggaacaacacatcaagaaagatcaacatcactggtatgcagatggtgccaaagattaacccatcaaaggcctttgtcggtagctccaacacctcctccttcacccccgtctctattgatgaggatgaagttggcacctttgtgtgtggtaccacctttggcgcaccaattgcagctaccgccggtggaaatcttttcgacatgtacgtgcacgtcacctactctggcactgagaccgagctagaaaagcactataatgttaccaaccccttcccattcatggacaatatttccctcgagaataagaccaacttttttgaaaagagagtcgccgagtatcaacgtgcccaggtcatggcttctatcaataagatcaagaaggaccaacaaacccaagaaactggttctcctctcccaattctgactgcacctcctccagtctcttcctcatcatccgaacaagaagatgttgaagacggcgtcggggactacatcagttatgacgattttatcagtt

SEQ ID NO: 67 - DNA, 26 bp, synthetic construct
  misc_feature 1..26: note = Single strand DNA oligonucleotide
  source 1..26: mol_type = other DNA
ataacgatgatgaggacaaatataag

SEQ ID NO: 68 - DNA, 51 bp, synthetic construct
  misc_feature 1..51: note = Single strand DNA oligonucleotide
  source 1..51: mol_type = other DNA
ggatcctaatacgactcactataggataacgatgatgaggacaaatataag

SEQ ID NO: 69 - DNA, 21 bp, synthetic construct
  misc_feature 1..21: note = Single strand DNA oligonucleotide
  source 1..21: mol_type = other DNA
gttaccaaccccttcccattc

SEQ ID NO: 70 - DNA, 46 bp, synthetic construct
  misc_feature 1..46: note = Single strand DNA oligonucleotide
  source 1..46: mol_type = other DNA
ggatcctaatacgactcactatagggttaccaaccccttcccattc

SEQ ID NO: 71 - DNA, 23 bp, synthetic construct
  misc_feature 1..23: note = Single strand DNA oligonucleotide
  source 1..23: mol_type = other DNA
aatcgtcataactgatgtagtcc

SEQ ID NO: 72 - DNA, 48 bp, synthetic construct
  misc_feature 1..48: note = Single strand DNA oligonucleotide
  source 1..48: mol_type = other DNA
ggatcctaatacgactcactataggaatcgtcataactgatgtagtcc

SEQ ID NO: 73 - DNA, 48 bp, synthetic construct
  misc_feature 1..48: note = Single strand DNA oligonucleotide
  source 1..48: mol_type = other DNA
ggatcctaatacgactcactataggtatggatactgcaaccaagtgga

SEQ ID NO: 74 - DNA, 23 bp, synthetic construct
  misc_feature 1..23: note = Single strand DNA oligonucleotide
  source 1..23: mol_type = other DNA
ctcgaaaacacgttccttaaacc

SEQ ID NO: 75 - DNA, 23 bp, synthetic construct
  misc_feature 1..23: note = Single strand DNA oligonucleotide
  source 1..23: mol_type = other DNA
tatggatactgcaaccaagtgga

SEQ ID NO: 76 - DNA, 48 bp, synthetic construct
  misc_feature 1..48: note = Single strand DNA oligonucleotide
  source 1..48: mol_type = other DNA
ggatcctaatacgactcactataggctcgaaaacacgttccttaaacc

SEQ ID NO: 77 - DNA, 250 bp, synthetic construct
  misc_feature 1..250: note = VP19-VP28 fusion PCR product
  source 1..250: mol_type = other DNA
ataacgatgatgaggacaaatataagaacaggaccagggatatgatgcttctggctgggtccgctcttctgttcctcgtttccgccgccaccgtttttatgtcttaccccaagaggaggcagtaaggcagaatctcacctttgagggaacattcaaggtgtggaacaacacatcaagaaagatcaacatcactggtatgcagatggtgccaaagattaacccatcaaaggcctttgtcggtagctccaac

SEQ ID NO: 78 - DNA, 250 bp, synthetic construct
  misc_feature 1..250: note = Rr2 PCR product
  source 1..250: mol_type = other DNA
gttaccaaccccttcccattcatggacaatatttccctcgagaataagaccaacttttttgaaaagagagtcgccgagtatcaacgtgcccaggtcatggcttctatcaataagatcaagaaggaccaacaaacccaagaaactggttctcctctcccaattctgactgcacctcctccagtctcttcctcatcatccgaacaagaagatgttgaagacggcgtcggggactacatcagttatgacgatt

SEQ ID NO: 79 - DNA, 250 bp, synthetic construct
  misc_feature 1..250: note = Wsv477 PCR product
  source 1..250: mol_type = other DNA
tatggatactgcaaccaagtggatggctgaaattattagagagaagaggggcaatattcaagaaataaaagtgacccctagagtagtcttcaatggcaatggttgtagtgcatgtttctctaacactaagagaaacttgtataactttggaacaaactataacaatgttgtacattgtgatttgttgtgcccttttgcaaggcataggattgtacatttcttataatggtttaaggaacgtgttttcgag

SEQ ID NO: 80 - DNA, 48 bp, synthetic construct
  misc_feature 1..48: note = Single strand DNA oligonucleotide
  source 1..48: mol_type = other DNA
ggatcctaatacgactcactataggtgggtaacaagattgatctggag

SEQ ID NO: 81 - DNA, 33 bp, synthetic construct
  misc_feature 1..33: note = Single strand DNA oligonucleotide
  source 1..33: mol_type = other DNA
acataggatcccattcaaggtgtggaacaacac

SEQ ID NO: 82 - DNA, 251 bp, synthetic construct
  misc_feature 1..251: note = synthetic DNA sequence encoding the VP28-RNAi polypeptide
  source 1..251: mol_type = other DNA
cattcaaggtgtggaacaacacatcaagaaagatcaacatcactggtatgcagatggtgccaaagattaacccatcaaaggcctttgtcggtagctccaacacctcctccttcacccccgtctctattgatgaggatgaagttggcacctttgtgtgtggtaccacctttggcgcaccaattgcagctaccgccggtggaaatcttttcgacatgtacgtgcacgtcacctactctggcactgagaccgag

SEQ ID NO: 83 - DNA, 33 bp, synthetic construct; identical to SEQ ID NO: 56
  misc_feature 1..33: note = Single strand DNA oligonucleotide
  source 1..33: mol_type = other DNA

SEQ ID NO: 84 - DNA, 31 bp, synthetic construct; identical to SEQ ID NO: 57
  misc_feature 1..31: note = Single strand DNA oligonucleotide
  source 1..31: mol_type = other DNA

SEQ ID NO: 85 - DNA, 33 bp, synthetic construct; identical to SEQ ID NO: 81
  misc_feature 1..33: note = Single strand DNA oligonucleotide
  source 1..33: mol_type = other DNA

SEQ ID NO: 86 - DNA, 31 bp, synthetic construct; identical to SEQ ID NO: 58
  misc_feature 1..31: note = Single strand DNA oligonucleotide
  source 1..31: mol_type = other DNA

SEQ ID NO: 87 - DNA, 676 bp, synthetic construct
  misc_feature 1..676: note = sequence of the complete VP28 RNAi fragment (Stem and loop)
  source 1..676: mol_type = other DNA
ggatcccattcaaggtgtggaacaacacatcaagaaagatcaacatcactggtatgcagatggtgccaaagattaacccatcaaaggcctttgtcggtagctccaacacctcctccttcacccccgtctctattgatgaggatgaagttggcacctttgtgtgtggtaccacctttggcgcaccaattgcagctaccgccggtggaaatcttttcgacatgtacgtgcacgtcacctactctggcactgagaccgagctgcagcgcatcttgcggttcttgcacgagcctactcacggtataaatcgagcgcgctccacccagactcatccatcgtagctcctaatgcggattcaacctggcgtttatatccgggaaaggtgcctaggagtgatagactcttaagtctttgggccatggctcggtctcagtgccagagtaggtgacgtgcacgtacatgtcgaaaagatttccaccggcggtagctgcaattggtgcgccaaaggtggtaccacacacaaaggtgccaacttcatcctcatcaatagagacgggggtgaaggaggaggtgttggagctaccgacaaaggcctttgatgggttaatctttggcaccatctgcataccagtgatgttgatctttcttgatgtgttgttccacaccttgaatgtctaga

SEQ ID NO: 88 - DNA, 21 bp, synthetic construct
  misc_feature 1..21: note = Single strand DNA oligonucleotide
  source 1..21: mol_type = other DNA
gggatacagctggtcaagaaa

SEQ ID NO: 89 - DNA, 22 bp, synthetic construct
  misc_feature 1..22: note = Single strand DNA oligonucleotide
  source 1..22: mol_type = other DNA
cgagagacttgaaggtattggg

SEQ ID NO: 90 - DNA, 26 bp, synthetic construct
  misc_feature 1..26: note = Single strand DNA oligonucleotide
  misc_feature 1..26: note = FAM labeled probe
  source 1..26: mol_type = other DNA
cgaggagctgattgttgtgttctcgt

SEQ ID NO: 91 - DNA, 22 bp, synthetic construct
  misc_feature 1..22: note = Single strand DNA oligonucleotide
  source 1..22: mol_type = other DNA
gtggagaccttccaacagtatg

SEQ ID NO: 92 - DNA, 22 bp, synthetic construct
  misc_feature 1..22: note = Single strand DNA oligonucleotide
  source 1..22: mol_type = other DNA
ccttcttgttgacctccttgat

SEQ ID NO: 93 - DNA, 21 bp, synthetic construct
  misc_feature 1..21: note = Single strand DNA oligonucleotide
  misc_feature 1..21: note = FAM labeled probe
  source 1..21: mol_type = other DNA
tgcgtgacatgaagcagacgg

SEQ ID NO: 94 - RNA, 588 nt, Penaeid shrimp infectious myonecrosis virus
  source 1..588: mol_type = genomic RNA; note = IMNV partial residue 473-1060 (dsRNA binding motif)
atgtctcaaacaaagaataaagaacctgttgttgaacagcaaaatcaacaaaccttcaaccaaaaagaccacaatgcgctagtgattgaacacaaaactagtgcaacaacgagcgcccaaagcattcccgttcgtccttcacgtaagaaggatgactatgtggacatgacacagggcaatttcaacctaattctaaaactgttaccaacaatttctgaactacagaagaggcatattcttcatctacttcgagaagaagtagaaggaaagaaagtttgtttcgtaaagcgagaaaaacagaatccacttatggctataaacgaactagccgttaaagttggagagaagcctaaatacacatcgacgaaaaccggagctgaccacattccaagctggactgtattggttgagttcgcaggttttagcgaagcagcgacatgtgacacagttaaaaacgcaaaaatgattgctgcttacaaattagttaaaagattttgtaaatgggacccaacctacattgaaatttctgattgtatgctgccacctccagaccttacatcgtgcggggacgttgagagtaatcctgga

(19) United States
(12) Patent Application Publication
(10) Pub. No.: US 2025/0108067 A1
(43) Pub. Date: Apr. 3, 2025

(54) COMPOSITIONS FOR AQUACULTURING

(71) Applicant: ViAqua Therapeutics Ltd., Haifa (IL)

(72) Inventors: Shai UFAZ, Givat Ada (IL); Ami SCHLESINGER, Kibbutz MaAgan Michael (IL); Ori KOSHET, Kibbutz Kfar HaMaccabi (IL); Margarita KANTEEV, Tirat Carmel (IL)

(73) Assignee: ViAqua Therapeutics Ltd., Haifa (IL)

(21) Appl. No.: 18/708,618

(22) PCT Filed: Nov. 8, 2022
(86) PCT No.: PCT/IL2022/051185

§ 371 Date: May 9, 2024

Related U.S. Application Data
(60) Provisional application No. 63/277,176, filed on Nov. 9, 2021.

(51) Int. Cl. A61K 31/713; A61K 9/00; A61K 9/14; A61K 31/7105; C12N 15/113

(52) U.S. Cl. CPC A61K 31/713; A61K 9/0056; A61K 9/146; A61K 31/7105; C12N 15/113; C12N 2310/14

(57) ABSTRACT

Particulate compositions comprising low molecular weight chitosan and ss or dsRNA partially complementary to, binding to or at least 90% identical to mRNA targets of viruses pathogenic in farmed crustaceans, compositions and farmed aquatic crustaceans comprising the same, and methods for their use in treating or preventing viral infection in aquaculture are provided.

### RELATED APPLICATION

This application claims the benefit of priority of U.S. Provisional Patent Application No. 63/277,176 filed on 9 Nov. 2021, the contents of which are incorporated herein by reference in their entirety.

### SEQUENCE LISTING STATEMENT

The Sequence listing file, entitled 94063.xml, created on Nov. 8, 2022, comprising 159,744 bytes, submitted concurrently with the filing of this application is incorporated herein by reference.

### FIELD AND BACKGROUND OF THE INVENTION

The most significant diseases injurious to the aquaculture industry are caused by infectious agents. Of these, the majority are viral diseases. The most common viral diseases affecting shrimp aquaculture include White Spot Syndrome Virus (WSSV), YellowHead Disease Virus (YHV), Taura Syndrome Virus (TSV), Infectious Hypodermal and Hematopoietic Necrosis Virus (IHHNV) and Monodon Baculovirus Disease (MBV), causing estimated billions of dollars in losses worldwide. Despite the urgent industry need for them, anti-viral methods for controlling shrimp viral pathogens are mostly unavailable due, in part, to lack of an adaptive immune response in crustaceans that renders conventional vaccination methods ineffective

RNAi gene silencing methods were first described for successful protection of shrimp against white spot disease (WSD) by injecting dsRNA specific to genes of white spot syndrome virus (WSSV) into shrimp in the laboratory as early as 2005.

Despite the proven efficacy of anti-viral RNAi technology, to date no method for use of dsRNA in shrimp farms has seen widespread acceptance in the industry. Most likely this is the result of the lack of simple and cost-effective delivery methods for farm-scale anti-viral RNAi methodologies. Recent studies on use and delivery of dsRNA to shrimp via injection and oral routes in hatcheries and on farms, have employed oral delivery using dsRNA-expressing bacteria as a component of dry feed pellets or use of living brine shrimp (Artemia) pre-fed with dsRNA before they are fed to shrimp. Also tested have been dsRNA enclosed in the chitosan-RNA particles including chitosan, liposomes and viral-like particles (VLP, empty, non-infective viral capsids) for direct injection or use as components of feed pellets for hatchery or pond-reared shrimp. Objective challenges to effective delivery of RNAi agents in feed include vulnerability to chemical and enzymatic degradation, leakage to the aqueous environment, and its opposite concern: limited bioavailability of encapsulated RNAi agents, as well as palatability, simplicity of operation and cost-effectiveness.

US Patent Publication No. 2014/0371295 to Loy et al teaches the delivery of dsRNA targeting organisms pathogenic to shrimp and other aquatic invertebrates (specifically the Myonecrosis virus) by feeding, injection, biolistic delivery, immersion, poration, liposomes, alphavirus replicon particles and various forms of encapsulation. US Patent Publication No. 2005/0080032 to Gross et al teaches delivery of dsRNA targeting pathogenic/parasitic microorganisms of marine invertebrates by injection, ingestion, immersion, encapsulation, specifically by microbial biodelivery of genetically engineered microorganisms expressing the dsRNA. US20200032267—to Sayre et al teaches RNAi strategies for biocontrol of aquatic pathogens in aquatic organisms using paratransgenic probiotic bacteria for RNAi delivery. US20150240236—to Brown et al teaches the use of viral-derived double-stranded RNA particles for delivery of RNAi targeting aquatic pathogens, and in particular, viral pathogens of salmonid fish and penaeid shrimp. Additional relevant publications include Itsathitphaisarn et al, J. Invert. Pathol, 2017, 147:76-85, US 20190175518 to Ufaz et al, US 2013/0245091 and U.S. Pat. No. 9,011,919 to Rozema et al, US 2014/0335192 to Ward et al, US 2011/0033547 to Kjems et al., PCT Publication No: WO 2008/003329 to Besenbacher et al, US Patent Publication 20080194504 to Kyle et al, EP Patent No. 2397123 to Aarhus Univ, US201020295355-A1 to Baker Shenda et al, US20110064664-A1 to Lopez-Berestein et al, US20120238735-A1 to McManus et al; Sarathi et al, Marine Biotech 2008 10.3:242-249; Feroskhan et al, Current Nanoscience 2014, 10.3:453-464.

### SUMMARY OF THE INVENTION

According to an aspect of some embodiments of the present invention there is provided a particulate composition comprising chitosan and at least one type of RNA molecule comprising a nucleic acid sequence at least 50 bases in length, wherein the chitosan is 50-100% deacetylated low molecular weight chitosan, wherein the chitosan:RNA ratio (w/w) of the particle is in the range of 0.3-0.9, and wherein the RNA molecule is capable of silencing expression of a gene when administered to an organism expressing the gene.

According to an aspect of some embodiments of the present invention there is provided a method for producing a particulate chitosan-RNA composition comprising:
i) rapidly adding an RNA solution to a volume of 50-100% deacetylated low molecular weight chitosan solution, wherein the chitosan solution comprises 2-20 mg/ml low molecular weight chitosan in 0.01-0.1 M organic acid,
ii) desalting and concentrating the chitosan-RNA complex solution, and
iii) drying the desalted, concentrated chitosan-RNA complex solution,
thereby producing the particulate chitosan-RNA composition.

According to an aspect of some embodiments of the present invention there is provided a nutriceutical composition comprising farmed crustacean food and the particulate composition described herein.

According to an aspect of some embodiments of the present invention there is provided a farmed crustacean comprising the particulate composition as described herein or the nutriceutical composition described herein.

According to an aspect of some embodiments of the present invention there is provided a farmed crustacean comprising the particulate composition as described herein.

According to an aspect of some embodiments of the present invention there is provided a farmed crustacean comprising the nutriceutical composition as described herein.

According to an aspect of some embodiments of the present invention there is provided a method of treatment or prevention of a disease or condition in a farmed crustacean associated with a pathogenic virus, the method comprising feeding the farmed crustacean the particulate composition described herein or the nutriceutical composition described herein, wherein the at least one type of RNA molecule is targeted to a gene product of the pathological virus, and wherein ingestion of the particulate composition by the farmed crustacean results in reduction in the level of a pathological virus in the farmed crustacean, compared to the same farmed crustacean ingesting feed devoid of RNA targeted to a gene product of the pathological virus.

According to some embodiments of the present invention the chitosan solution of (i) comprises 2-20 mg/ml low molecular weight chitosan in 0.02 M organic acid.

According to some embodiments of the present invention the RNA solution comprises 0.2-10 mg/ml RNA in water.

According to some embodiments of the present invention the RNA solution is added to the chitosan solution in a ration in the range of 1.0:0.14 to 1:1 volumes RNA:chitosan.

According to some embodiments of the present invention the RNA solution is added to the chitosan solution in a ratio of 1:1 volumes RNA:chitosan.

According to some embodiments of the present invention the organic acid is selected from the group consisting of acetic acid, lactic acid, propionic acid and citric acid.

According to some embodiments of the present invention the organic acid is citric acid.

According to some embodiments of the present invention the RNA solution comprises 3 mg/ml RNA in water.

According to some embodiments of the present invention the chitosan solution comprises 3 mg/ml low molecular weight chitosan in 0.02 M organic acid.

According to some embodiments of the present invention the chitosan:RNA ratio of the particulate composition is in the range of 0.4-0.8 (w/w).

According to some embodiments of the present invention the chitosan:RNA ratio of the particulate composition is in the range of 0.5-0.9 (w/w).

According to some embodiments of the present invention the chitosan:RNA ratio of the particulate composition is in the range of 0.5 (w/w).

According to some embodiments of the present invention the molecular weight of the chitosan of the particulate composition is in the range of 10-50 kDa.

According to some embodiments of the present invention the molecular weight of the chitosan of the particulate composition is about of 30 kDa.

According to some embodiments of the present invention the chitosan:RNA ratio of the particulate composition is in the range of 0.45-0.55 and the molecular weight of the chitosan of the particulate composition is in the range of 20-30 kDa.

According to some embodiments of the present invention the particulate composition comprises >50% particles in the range of 10-300 μm.

According to some embodiments of the present invention the particulate composition has a Gaussian particle size distribution.

According to some embodiments of the present invention the at least one type of RNA molecule is a single stranded RNA (ssRNA).

According to some embodiments of the present invention the at least one type of RNA molecule is a double-stranded RNA (dsRNA).

According to some embodiments of the present invention the particulate composition provides enhanced survival of shrimp upon feeding of the particulate composition.

According to some embodiments of the present invention the RNA content (w/w) of the nutriceutical composition is in the range of 0.01-10%.

According to some embodiments of the present invention the RNA content (w/w) of the nutriceutical composition is in the range of 0.05-1.0%.

According to some embodiments of the present invention the RNA content (w/w) of the nutriceutical composition is in the range of 0.1-0.5%.

According to some embodiments of the present invention the organic acid is selected from the group consisting of acetic acid, lactic acid and propionic acid.

According to some embodiments of the present invention the RNA solution comprises 3 mg/ml RNA in water.

According to some embodiments of the present invention the method employs chitosan-RNA particles of the particulate composition as described herein.

According to some embodiments of the present invention the nutriceutical compositions are prepared according to the method described herein.

According to some embodiments of the present invention, ingestion of the particulate composition or nutriceutical composition by the farmed crustacean results in increased survival, yield, growth rate, vigor, biomass, feed conversion, size, quality of taste and odor or stress tolerance of the farmed crustacean, compared to the same farmed crustacean ingesting feed devoid of RNA targeted to a gene product of the pathological virus. According to some embodiments of the present invention the farmed crustaceans are selected from the group consisting of Shrimp, Prawns, Crabs, Lobsters and Crayfishes. In some embodiments the farmed crustaceans are shrimp or prawns.

According to some embodiments of the present invention the virus is selected from the group consisting of White Spot Syndrome Virus (WSSV, Accession No. AF332093), Taura syndrome virus (TSV, Accession No. NC_003005), Yellow head virus (YHV, Accession No. FJ848673.1), Gill-associated virus (GAV, Accession No. NC_010306.1), Infectious hypodermal and hematopoietic necrosis virus (IHHNV, Accession No. NC_002190), Infectious myonecrosis virus (IMNV, Accession No. KR815474.1), White Tail Disease (Macrobrachium rosenbergii nodavirus, MrNV, Accession No. NC_005094.1 (segment 1) and NC_005095.1 (segment 2)), Infectious pancreatic necrosis virus (IPNV, Accession No. NC_001915.1 (Segment A) and NC_001916.1 (Segment B)) and Decapod iridescent virus 1 (DIV1) Accession No. MF599468.

According to some embodiments of the present invention the at least one type of RNA molecule comprises one or more of the following:
(a) at least one sequence capable of binding through complementary base pairing to a target mRNA molecule of a virus pathogenic in farmed crustaceans;
(b) at least one sequence having at least 90% sequence identity to a target mRNA molecule of a virus pathogenic in farmed crustaceans;
(c) at least one sequence at least partially complementary to a target mRNA molecule of a virus pathogenic in farmed crustaceans;
(d) at least one sequence identical to at least 21 contiguous bases of a target mRNA molecule of a virus pathogenic in farmed crustaceans;
(e) at least one sequence capable of binding through complementary base pairing to a target mRNA molecule of a farmed crustacean;
(f) at least one sequence identical to at least 21 contiguous bases of a target mRNA molecule of a farmed crustacean;
(g) at least one sequence at least partially complementary to a target mRNA molecule of a farmed crustacean.

According to some embodiments of the present invention the partially complementary sequence or sequence capable of binding through complementary base pairing to the target nucleic acid molecule of the nucleotide sequence is complementary to a sequence of the target nucleic acid molecule at least 50, at least 75, at least 100, at least 150, at least 200, at least 250, at least 400, at least 500, at least 600, at least 750 bases in length.

According to some embodiments of the present invention the at least one RNA molecule comprising at least one sequence identical to at least 21 contiguous bases of a target mRNA molecule of the pathogenic virus in farmed crustaceans is identical to at least 50, at least 75, at least 100, at least 150, at least 200, at least 250, at least 400, at least 500, at least 600, at least 750 bases of the target mRNA molecule.

According to some embodiments of the present invention the RNA molecule comprising at least one sequence capable of binding through complementary base pairing to a target mRNA molecule of a virus pathogenic in farmed crustaceans or comprising at least one sequence at least partially complementary to a target mRNA molecule of a virus pathogenic in farmed crustaceans comprises:
(a) a nucleic acid sequence complementary to a nucleic acid sequence selected from the group consisting of SEQ ID NO: 1, 2, 3 and 5, or
(b) a nucleic acid sequence complementary to a nucleic acid sequence selected from the group consisting of SEQ ID NOs: 1, 2, 3 and 5.

According to some embodiments of the present invention the RNA molecule comprising at least one sequence having at least 90% sequence identity to a target mRNA molecule of a virus pathogenic in farmed crustaceans or at least one sequence identical to at least 21 contiguous bases of a target mRNA molecule of a virus pathogenic in farmed crustaceans comprises:
(a) a nucleic acid sequence identical to at least 21 contiguous bases of a nucleic acid sequence selected from the group consisting of SEQ ID NO: 59, 64, 65, 77, 78 and 79, or
(b) a nucleic acid sequence complementary to a nucleic acid sequence selected from the group consisting of SEQ ID NOs: 59, 64, 65, 77, 78 and 79.

According to some embodiments of the present invention the particulate composition, nutriceutical composition or method as described herein comprises at least one additional RNA sequence capable of directing cleavage of a target mRNA molecule of a virus pathogenic in farmed crustaceans.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a typical chromatogram of dsRNA targeting the Rab7 gene, extracted from the bacterial synthesis according to the methods of the invention;

FIG. 2 is a histogram showing Rab7 silencing by feeding of Rab7-targeted chitosan:dsRNA particles, using a 30 kDa molecular weight chitosan polymer, chitosan:RNA mass ratios (1:1 to 1:2), prepared with feed in an RNA content of 2% of the feed volume, dried at high temperatures (80-120° C.) and provided as feed to the shrimp. RNA was extracted from gills of shrimp after 4 days feeding with the feed-chitosan:RNA complex. Rab7 expression was measured by quantitative PCR after extraction and purification, and expressed as mRNA level relative to shrimps fed on feed without dsRNA (negative control). Note the superior silencing of Rab7 expression by feeding the chitosan-RNA particles, compared to injected dsRNA, and thus enhanced stability of particles with the lower (1:2) chitosan:RNA ratio at all temperatures. Injection of 1 μg dsRNA per 1 gram shrimp served as a positive control;

FIG. 3 is a histogram showing Rab7 silencing by feeding of Rab7-targeted chitosan:dsRNA particles prepared with different organic acids. The chitosan:dsRNA complexes were prepared at a ratio of 1:1 with acetic, lactic, and citric acids. Shrimp feed containing 2% dsRNA was provided as feed to the shrimp. RNA was extracted from gills of shrimp after 4 days feeding with the feed-chitosan:RNA complex. Rab7 expression was measured by quantitative PCR after extraction and purification, and expressed as mRNA level relative to shrimps fed on feed without dsRNA (negative control). Note the superior silencing with the chitosan:dsRNA particles prepared with lactic and citric acid, compared to those prepared with acetic acid;

FIG. 4 is a gel showing electrophoretic analysis of integrity and stability of chitosan:dsRNA particles prepared at different chitosan:dsRNA ratios. Chitosan:dsRNA particles of different ratios were prepared and separated on the gel to visualize dsRNA retention. Non-complexed dsRNA was evaluated by centrifuging the complexes and visualizing the dsRNA released to the supernatant. Lanes 1 and 6: DNA ladder 100 bp. Lanes 2-5: chitosan:dsRNA complexes at ratios of 0.25:1, 0.5:1, 1:1 and 4:1 (w:w) respectively. Lanes 7-10: supernatant of chitosan:dsRNA complexes at ratios of 0.25:1, 0.5:1, 1:1 and 4:1 (w:w) respectively. Note superior retention of dsRNA with higher chitosan:dsRNA rations (lanes 4 and 5), and the absence of free dsRNA detected in the supernatants (Lanes 7-10);

FIGS. 5A and 5B are gels showing electrophoretic analysis of resistance of chitosan:dsRNA particles prepared at different chitosan:dsRNA ratios to RNase digestion. FIG. 5A: Chitosan:dsRNA particles of different chitosan:dsRNA ratios were treated with RNase and analyzed on agarose gels to visualize degradation of RNA. FIG. 5A and FIG. 5B: Lane 1: DNA ladder 100 bp, lane 2—untreated naked dsRNA, lane 3—naked dsRNA, lanes 4-7: complexes at chitosan:dsRNA ratio of 0.25:1, 0.5:1, 1:1 and 4:1 (w:w) respectively. Separation of complexed RNA extracted from the RNase-treated chitosan:dsRNA particles (FIG. 5B) shows enhanced protection with higher chitosan:dsRNA rations (lanes 5, 6 and 7);

FIG. 6A is a chart showing the particle size distribution for chitosan:RNA particles at 1:1 chitosan:RNA mass ratio, measured by the Mastersizer 2000 Laser Diffraction Particle Size Analyzer (Malvernpananalytical, Malvern, UK). Note the Gaussian size distribution, with an average mean of 57.8 μm;

FIG. 6B is a chart showing the particle size distribution for chitosan:RNA particles at 1:2 chitosan:RNA mass ratio, measured by the Mastersizer 2000 Laser Diffraction Particle Size Analyzer (Malvernpananalytical, Malvern, UK). Note the Gaussian size distribution, with an average mean of 49.0 μm;

FIG. 7A is a chart showing the particle charge for chitosan:RNA particles at 1:1 chitosan:RNA mass ratio (zeta potential of 28.7 mV), measured by the DLS ZETASIZER NANO ZSP Analyzer (Malvernpananalytical, Malvern, UK);

FIG. 7B is a chart showing the particle size distribution for chitosan:RNA particles at 1:2 chitosan:RNA mass ratio (zeta potential of −16.4 mV), measured by the DLS ZETASIZER NANO ZSP Analyzer (Malvernpananalytical, Malvern, UK);

FIG. 8 is a histogram showing the effect of chitosan molecular weight on Rab7 silencing by feeding of Rab7-targeted chitosan:dsRNA particles. The chitosan:dsRNA complexes were prepared at a ratio of 1:1, 1:2, 2:1 and 4:1 with either 30 KDa or 200 KDa chitosan. Shrimp feed containing 5% dsRNA was provided as feed to the shrimp. RNA was extracted from gills of shrimp after 5 days feeding with the feed-chitosan:RNA complex. Rab7 expression was measured by quantitative PCR after extraction and purification, and expressed as mRNA level relative to shrimps fed on feed without dsRNA (negative control). Note the superior bioavailability with particles of 1:2 and 1:1 chitosan:dsRNA mass ratio, negatively affected by the increased molecular weight chitosan particles prepared with lactic and citric acid, compared to those prepared with acetic acid;

### DESCRIPTION OF SPECIFIC EMBODIMENTS OF THE INVENTION

The present invention, in some embodiments thereof, relates to methods for producing chitosan-RNA particles comprising ssRNA and dsRNA and, more particularly, but not exclusively, to the use of same for increasing resistance of farmed aquatic crustaceans to viruses. In particular, the present invention provides modified chitin-RNA particles and methods for enhancing resistance to infection by viral pathogens of farmed aquatic crustaceans, such as shrimp and prawns.

The principles and operation of the present invention may be better understood with reference to the drawings and accompanying descriptions.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Application of gene silencing technology to commercial farming has generated great interest, providing a possible means of compensating for the loss of genetic variation which is common to wild species, but greatly diminished in the process of domestication and inbreeding of commercial species. However, development of means for efficient and cost-effective delivery of effective amounts of RNA silencing agents to host organisms has proven elusive.

Aquaculture, or farming or culturing of aquatic species, is an age-old but also fast-growing industry, of particular importance recently in light of dwindling numbers of wild fresh and salt water stocks, and the increased prominence of fish and seafood in the Western diet. Commonly cultured aquatic species now include fish, crustaceans, mollusks and even seaweed and echinoderms (sea cucumbers).

Along with the advantages of farming, high-density culture of aquatic populations, and the limited genetic variability of the farmed populations have created an opportunity for pathogenic organisms, and particularly viral pathogens, to flourish and sometimes reach epidemic proportions, resulting in loss of income and significant reduction in stock populations.

While gene silencing by RNAi may be an attractive solution to the challenges of providing therapeutic treatment in an aquaculture environment (e.g. dsRNA can be amplified within the host organism), methods for effective and cost-effective delivery of RNAi agents to cultured aquatic species are yet to be developed. In vivo gene silencing studies with chitosan-RNA particles as the delivery platform have met with little success.

Whilst reducing the present invention to practice, the present inventors have developed chitin-based compositions capable of efficiently binding and delivering RNAi species. The inventors have identified, through rigorous experimentation, specific modifications of the chitosan-RNA particle, and processes for its production, which can be used to produce chitosan-RNA particles comprising double stranded RNA (dsRNA) and/or single stranded RNA (ssRNA) which, when ingested by or administered to shrimp, effectively deliver the RNA to the cells of the host organism (silencing the RNA-targeted gene expression) (see Example I, FIGS. 2, 3 and 8), thus providing an efficient means for silencing viral and/or host genes. Specifically, the instant inventors have uncovered that selecting low-molecular weight, highly deacetylated chitosan, greatly reducing the chitosan:RNA ratio and unique conditions for chitosan-RNA processing significantly enhances the formation of chitosan-RNA particles bearing greater amounts of RNAi, results in particle preparations exhibiting a surprising uniformity of size; particles which are stable and effectively shield the RNA from degradation in aqueous environments. Surprisingly, the chitosan-RNA particles provided enhanced bioavailability of the RNAi payload, and were equally effective in delivering dsRNA to host organisms orally, when provided in feed, or through direct administration (e.g. injection).

Thus, according to some embodiments of aspects of the present invention there is provided a particulate composition comprising chitosan and at least one type of RNA molecule comprising a nucleic acid sequence at least 50 bases in length, wherein the chitosan is 50-100% deacetylated low molecular weight chitosan, wherein the chitosan:RNA ratio (w/w) of said particle is in the range of 0.3-0.9, and wherein the RNA molecule is capable of silencing expression of a gene when administered to an organism expressing the gene.

The term “chitin”, as used herein refers to a long-chain biopolymer of N-acetylglucosamine [i.e. 2-(acetylamino)-2-deoxy-D-glucose] in beta-1,4 linkage [poly (N-acetyl-1,4-beta-D-glucopyranosamine)]. Chitin is ubiquitous in insect exoskeletons and fungi and bacterial cell walls, and abundantly available commercially (see, for example, Sigma-Aldrich product No. C7170, Sigma-Aldrich, St Louis, MO). A distinction is made between alpha, beta and gamma chitin, which differ in the arrangement of their polymer chains (and, subsequently, in their mechanical properties): alpha chitin has alternating antiparallel arrangement of the chains (most common in crustaceans); beta chitin has a parallel arrangement of the polysaccharide chains (common in squids) and gamma chitin has two parallel chains statistically alternating with an antiparallel chain (common in fungi). The term “chitin” is synonymous with any one of alpha, beta or gamma chitin, unless otherwise indicated. Chitin can be natural chitin, from naturally chitin-containing organisms, recombinant chitin from organisms genetically engineered to produce chitin or synthetic, chemically synthesized chitin from polymerization of mixtures of glucosamine monomers or oligomers. Different types of chitin polymer arrangements are distinguishable, for example, by proton nuclear magnetic resonance (1H NMR) spectroscopy and matrix-assisted laser desorption ionization time-of-flight mass spectrometry (MALDI-TOF MS).

Chitin is fairly insoluble, dissolving only in strong solvent systems.

As used herein, the term “chitosan” refers to a deacetylated chitin polymer, which, in contrast to chitin, has free amino groups. Chitosan can be obtained by alkaline deacetylation of chitin, producing a chitin polymer with randomly acetylated amine groups, with a variety of properties depending on the degree of deacetylation and chain length. Chitosan is safe, non-toxic, easily dissolved in weak acidic solutions and can interact with polyanions to form complexes and gels. Chitosan can be characterized, inter alia, by the ratio of acetylated to non-acetylated (deacetylated) amine groups in the polymer, and by the average chain length of the polymers. Commercially available chitosan is usually provided as a powder, in alpha, beta or gamma orientation (according to the source of the chitin), with the degree of deacetylation (% DD) between 60 and 100%, and a molecular weight between about 4 to greater than 250 kDa, and in various degrees of purity. As used herein, the term “partially deacetylated chitosan” refers to chitosan which has been treated to acetylate a portion of the deacetylated, free amino groups. As in commercially available chitosan, partially deacetylated chitosan is characterized by the degree of deacetylation (% DD). The degree of deacetylation of chitosan can be determined by a variety of methods, usually classified into three categories: (1) spectroscopy (IR, (1)H NMR, (13)C NMR, (15)N NMR, and UV); (2) conventional (various types of titration, conductometry, potentiometry, ninhydrin assay, adsorption of free amino groups of chitosan by pictric acid) and (3) destructive (elemental analysis, acid or enzymatic hydrolysis of chitin/chitosan and followed by the DA measurement by colorimetry or high performance liquid chromatography, pyrolysis-gas chromatography, and thermal analysis using differential scanning calorimetry) methods.

According to some aspects of the invention, the particulate composition comprises untreated chitosan, i.e. chitosan which has not been treated to reduce the degree of deacetylation. It will be appreciated that the degree of deacetylation of a chitosan preparation relates to the distribution of chitosan molecules within the population of chitosan being used for the methods and compositions of the invention. Thus, the chitosan of the particulate composition of the invention can be characterized as being deacetylated within a range of values for degree of deacetylation. In particular embodiments, the particulate composition comprises chitosan having a degree of deacetylation in the range of 65-100%, in the range of 70-100%, in the range of 75-100%, in the range of 80-100%, in the range of 85-100%, 90-100%, 95-100%, in the range of 65-95%, 71-91%, 75-89% or 78-85%. In other embodiments the particulate composition comprises chitosan having a degree of deacetylation of 65%, 67%, 70%, 72%, 74%, 76%, 78%, 80%, 82%, 84%, 86%, 88%, 90%, 91%, 92%, 93%, 94%, 95%, 96%, 97%, 98%, 99% or 100%. In specific embodiments of the invention the particulate composition comprises chitosan having a degree of deacetylation of 75-100%. In other embodiments, the particulate composition comprises chitosan having a degree of deacetylation of 80-95%. In further embodiments, the particulate composition comprises chitosan having a degree of deacetylation of 90%.

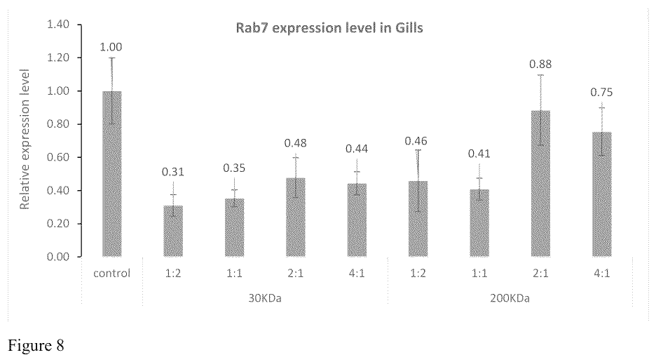

While many methods using chitosan for delivering RNAi operate in a range of chitosan:RNA ratio of 8, 10, 20, 40 or greater, the present inventors have shown that formulating the particulate composition comprising the chitosan-RNAi using a uniquely low chitosan:RNA mass ratio (<1.0) results in a particulate composition comprising chitosan-RNAi particles extremely effective in host gene silencing (see FIGS. 2 and 8), as compared with particulate compositions comprising chitosan-RNAi particles having a higher (>1.0) chitosan:RNA mass ratio (see FIG. 8). While not wishing to be limited to a single hypothesis, the present inventors believe that the superior effectiveness of particles having lower chitosan:RNA mass ratio not only increases the amount of RNAi fed per mass of particles in the feed, but also improves the palatability, to the shrimp, of the particle-containing feed.

Further, the inventors have shown that particulate compositions comprising the chitosan-RNAi using the low chitosan:RNA mass ratio (<1.0; 0.5) results in a particulate composition comprising chitosan-RNAi particles with enhanced thermal stability (see FIG. 2). Thus, in some embodiments, the RNAi of the particulate composition comprising chitosan-RNAi particles having a low (<1.0) chitosan:RNA mass ratio has improved resistance to degradation compared to particulate compositions comprising chitosan-RNAi particles of a high (>1.0) chitosan:RNA mass ratio. In some embodiments, the RNA of the low chitosan:RNA mass ratio particles are 10, 15, 20, 25, 30, 35, 40, 45, 50, 55, 60% or more resistant to thermal degradation than that of the higher (>1.5) chitosan:RNA mass ratio particles.

Thus, in some embodiments of some aspects of the invention, the particulate composition comprises particles having a chitosan:RNA ratio (w/w) in the range of 0.2-0.95. In some embodiments, the chitosan:RNA ratio is in the range of 0.25-0.9, 0.3-0.85, 0.35-0.80, 0.4-0.9, 0.3-0.8, 0.2-0.75 and 0.25-0.5. In other embodiments, the chitosan:RNA ratio of the particles is about 0.2, 0.25, 0.30, 0.35, 0.40, 0.45, 0.50, 0.55, 0.60, 0.65, 0.70, 0.75, 0.80, 0.85, 0.90, 0.95. In specific embodiments, the particulate composition comprises particles having a chitosan:RNA ratio (w/w) of 0.45-0.55, and, in particular, chitosan:RNA ratio (w/w) of 0.5. In other specific embodiments, the chitosan is 75-100% deacetylated, low molecular weight chitosan, and the chitosan:RNA mass ratio (w/w) of the particle is in the range of 0.3-0.9. In further embodiments, the chitosan is 80-95% deacetylated, low molecular weight chitosan, and the chitosan:RNA mass ratio (w/w) of the particle is in the range of 0.45-0.55.

While reducing the invention to practice, the inventors have uncovered that particulate compositions comprising chitosan-RNAi particles formulated from low molecular weight chitosan provide significantly increased bioavailability of the RNAi payload, while complexing RNAi in particles with higher molecular weight chitosan impaired the bioavailability of the RNAi (FIG. 8). It will be appreciated that the molecular weight of a chitosan preparation relates to the distribution of molecular weights of the chitosan molecules within the population of chitosan being used for the methods and compositions of the invention.

Thus, in some embodiments, the molecular weight of the chitosan in the particulate composition is in the range of 5-100 kDa. In other embodiments, the molecular weight of the chitosan in the particulate composition is in the range of 10-85 kDa, in the range of 12-90 kDa, in the range of 15-75 kDa, 17-65 kDa, 20-60 kDa, 25-55 kDa, 20-50 kDa, 20-40 kDa, 23-38 kDa, 25-35 kDa, or 20-30 kDa. In still other embodiments, the chitosan is a low molecular weight chitosan, having a degree of deacetylation of about 10, 12, 14, 16, 18, 20, 22, 24, 26, 28, 30, 32, 34, 36, 38, 40, 42, 44, 46, 48, 50, 52, 55, 58, 60, 62, 65, 68, 70, 72, 75, 78, 80, 82, 85, 88, 90, 92, 95, 98, 99 or 100 kDa. In particular embodiments the the molecular weight of the chitosan in the particulate composition is in the range of 10-50 kDa, 20-30 kDa or 30 kDa.

In specific embodiments, the particulate composition of the invention comprises chitosan:RNA particles comprising 75-100% deacetylated chitosan, and having a chitosan:RNA ratio in the range of 0.45-0.55, or 0.5 and chitosan of a molecular weight in the range of 20-30 kDa, in some embodiments 30 kDa.

According to some aspects of the invention, the chitosan-RNA particles are particles, i.e. having a particle size within the micrometer (μm) rather than within the nanometer (nm) range. Particle size may be measured by z-average diameter, for example, using the Mastersizer (Malvernpanalytical, Ltd). Alternatively, particle size can be measured using microscopic techniques with microscopic resolution above 1 micron.

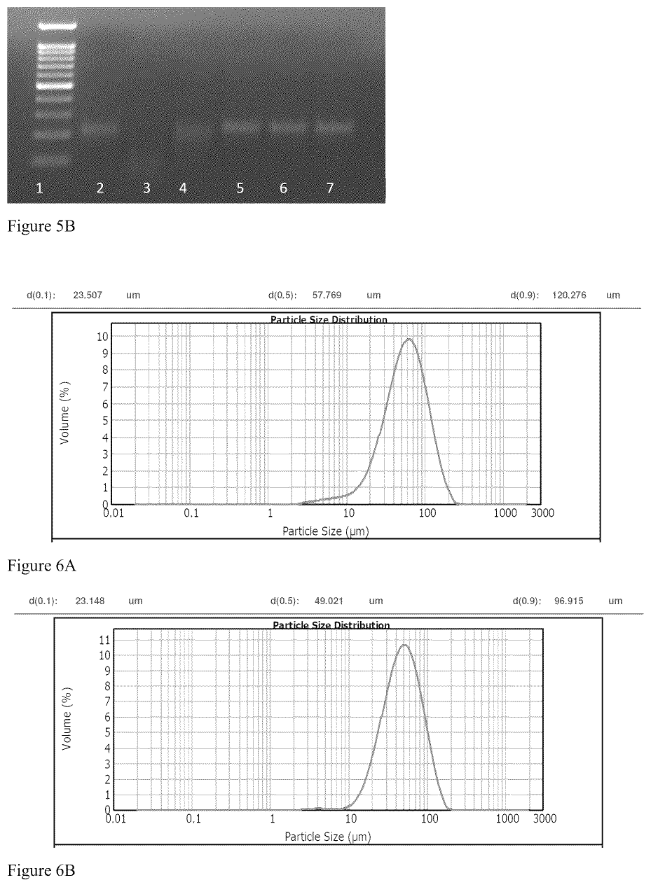

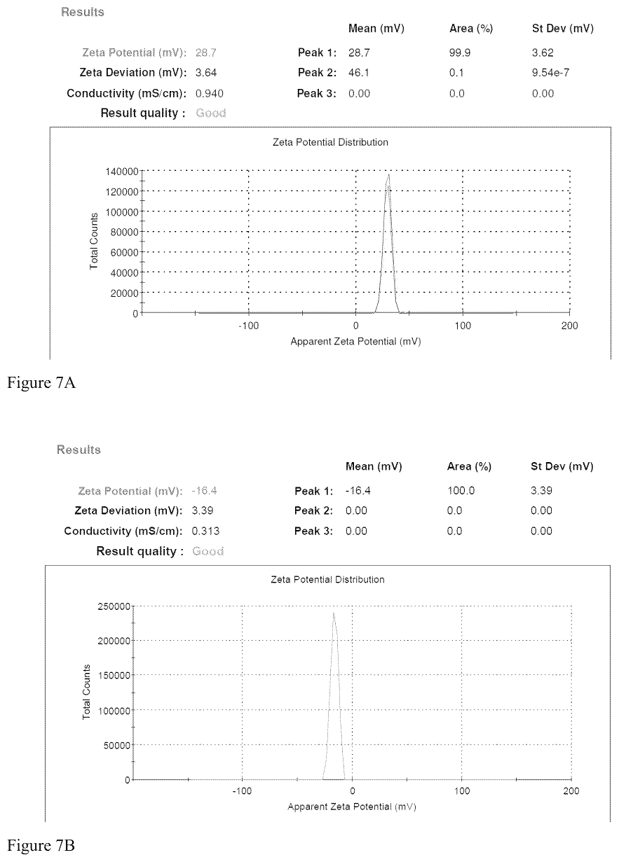

The present inventors have uncovered a correlation between the chitosan:RNA (w/w) mass ratio of the particles and the mean particle size of the particulate composition of the invention (see FIGS. 6A, 6B), with the mean particle size decreasing with reduction in the chitosan:RNA (w/w) mass ratio. Thus, in some embodiments, the particulate composition comprises greater than 50% of the particles in the range of 10-300 μm. In other embodiments, the chitosan-RNA particles of the composition have mean particle size of about 10, about 15, about 18, about 20, about 27, about 30, about 32, about 35, about 38, about 40, about 43, about 45, about 47, about 50, about 55, about 60, about 65, about 70, about 75, about 80, about 85, about 90, about 95, about 100, about 110, about 120, about 130, about 140, about 150, about 160, about 170, about 180, about 190, about 200, about 210, about 220, about 230, about 240, about 250, about 260, about 270, about 280, about 290 or about 300 μm.

The present inventors have noted that chitosan-RNA populations of the particulate composition of the invention, having the features as described herein and prepared according to the methods of the invention, exhibit a Gaussian, rather than a polyphasic particle size distribution (see FIGS. 6A, 6B). The uniformity of particle size likely contributes to the increased bioavailability, as well as the suitability for oral administration (in the feed) to shrimps. Thus, in some embodiments, the particulate composition of the invention has a Gaussian particle size distribution.

According to some embodiments of the invention the chitosan-RNA particle comprises at least one type of RNA. In some embodiments, the at least one type of RNA is a double stranded RNA (dsRNA). As used herein the term “dsRNA” relates to two strands of anti-parallel polyribonucleic acids held together by base pairing. “dsRNA” may also be described as an “dsRNA duplex” or “RNA duplex” or “duplex RNA”. The two strands can be of identical length or of different lengths provided there is enough sequence homology between the two strands that a double stranded structure is formed with at least 80%, 90%, 95% or 100% complementarity over the entire length. The level of complementation between the strands of dsRNAs suitable for use with the invention as described herein is selected to provide at least sufficient complementation to affect gene silencing of the target RNA molecules of interest.

In other specific embodiments of the invention, the chitosan-RNA particle comprises at least one single stranded RNA (ssRNA). As used herein the term “ssRNA” relates to a single strand of polyribonucleic acid. When the single stranded RNA is a silencing RNA directed to a specific target sequence in a target organism (e.g. shrimp sequence such as Rab7, or pathogen sequence such as WSSV VP28, VP19 or Rr2, etc), the ssRNA can be either identical to the target RNA sequence or a portion thereof (“Sense ssRNA”), or, alternatively, complementary to the target RNA sequence or a portion thereof (“Anti-sense ssRNA”). In some embodiments, the particles can comprise a combination of sense- and antisense ssRNA.

It will be noted that some single stranded RNA sequences may comprise self-complementary portions (e.g. “pallindromic” or “sub-sequences”) which, under certain conditions, can form segments of internal base pairing, resulting in partial double stranded RNA formation from the single-stranded RNA of the particles of the invention. It will be noted that the ss or dsRNA can be defined in terms of the nucleic acid sequence of the DNA encoding the target gene transcript, and it is understood that an antisense ss or dsRNA sequence corresponding to the coding sequence of a gene comprises an RNA complement of the gene's coding sequence, or other sequence of the gene which is transcribed into RNA.

Ss or dsRNA can be used for downregulation of gene expression by RNA silencing. As used herein, the phrase “RNA silencing” refers to a group of regulatory mechanisms [e.g. RNA interference (RNAi), transcriptional gene silencing (TGS), post-transcriptional gene silencing (PTGS), quelling, co-suppression, and translational repression] mediated by RNA molecules which result in the inhibition or “silencing” of the expression of a corresponding protein-coding gene, or, in some cases, gene encoding a functional RNA product. RNA silencing has been observed in many types of organisms, including plants, animals (vertebrates and invertebrates), and fungi.

The RNA can be a ss or dsRNA which is capable of specifically inhibiting or “silencing” the expression of a target gene. In some embodiments the RNA is capable of preventing complete processing (e.g. the full translation and/or expression) of an mRNA molecule through a post-transcriptional silencing mechanism. When used for gene silencing, RNA include noncoding RNA molecules, for example RNA duplexes comprising paired strands, as well as precursor RNAs from which such small non-coding RNAs can be generated. Exemplary RNA that can be used for RNA silencing include siRNA duplexes, miRNAs and shRNAs. In one embodiment, the RNA is capable of inducing RNA interference. In another embodiment, the RNA is capable of mediating translational repression. In yet another embodiment, the RNA is capable of directing cleavage of a target RNA. Cleavage of such a target RNA can be effected via an RNAi pathway, as described in detail hereinbelow.

The inhibitory RNA sequence of the RNA can be greater than 90% identical, or even 100% identical, to the portion of the target gene transcript. In some embodiments, the RNA, for example, the duplex region of the dsRNA may be defined functionally as a nucleotide sequence that is capable of binding through complementary base pairing to a target mRNA molecule of the virus—e.g. hybridizing with a portion of the target gene transcript under stringent conditions (e.g., 400 mM NaCl, 40 mM PIPES pH 6.4, 1 mM EDTA, 60 degrees C. hybridization for 12-16 hours; followed by washing).

In some embodiments, the inhibitory RNA sequence of the particle is a single stranded, sense RNA sequence, comprising at least one sequence having at least 90% sequence identity to the target gene transcript (e.g. target mRNA molecule).

In other embodiments, the inhibitory RNA sequence of the particle is a double stranded, sense RNA and complementary sequence, comprising at least one sequence having at least 90% sequence identity to the target gene transcript (e.g. target mRNA molecule).

The length of the double-stranded or single stranded nucleotide sequences complementary or identical to the target gene transcript may be at least about 18, 19, 20, 21, 25, 40, 50, 75, 100, 150, 200, 250, 300, 400, 491, 500, 600 or at least 750 or more bases (or base pairs, in the case of dsRNA). In some embodiments, the double-stranded or single stranded nucleotide sequences complementary or identical to the target gene transcript comprise at least 20, at least about 21, 25, 40, 50, 75, 100, 125, 150, 200, 250, 300, 400, 491, 500, 600 or at least 750 or more contiguous bases. In some embodiments, the length of the targeted mRNA sequence can be at least 20, at least 40, at least 50, at least 75, at least 100, at least 150, at least 200, at least 250, at least 400, at least 500, at least 600, at least 750 bases in length. It will be appreciated that each of the lengths represents a separate embodiment.

In some embodiments of some aspects of the invention, the length of the single stranded or double-stranded nucleotide sequence is approximately from about 40 to about 450, about 50 to about 450, about 60 to about 350, about 60 to about 300 and about 80 to about 250 nucleotides in length for genes of viruses of farmed aquatic crustaceans thereof. In some embodiments of some aspects of the invention, the length of the single or double-stranded nucleotide sequence is approximately from 50 to 350 nucleotides in length for genes of viruses of farmed aquatic crustaceans thereof. In specific embodiments, the length of the single or double-stranded nucleotide sequence is at least 50 nucleotides. In specific embodiments, the length of the single or double-stranded nucleotide sequence is 125 or 250. It will be appreciated that each individual range represents a distinct and separate embodiment.

The term “corresponds to” as used herein means a polynucleotide sequence possessing homology (or identity, for example, 100% homology) to all or a portion of a reference polynucleotide sequence. In contradistinction, the term “complementary to” is used herein to mean that the complementary sequence is homologous (or identical, i.e. 100% homologous) to all or a portion of the complement of a reference polynucleotide sequence. For example, the nucleotide sequence “TATAC” corresponds to a reference sequence “TATAC” and is complementary to a reference sequence “GTATA”.

The present teachings relate to various lengths of ss or dsRNA, whereby the shorter version (shorter or equal to 50 bp (e.g., 17-50)), is referred to as siRNA or miRNA. Longer ss or dsRNA molecules of 51-600 are referred to herein as ss or dsRNA, which can be further processed to siRNA molecules. According to some embodiments, the nucleic acid sequence of the ss or dsRNA is greater than 15 bases or base pairs in length. According to yet other embodiments, the nucleic acid sequence of the ss or dsRNA is 19-25 bases or base pairs in length, 30-100 bases or base pairs in length, 100-250 bases or base pairs in length or 100-500 bases or base pairs in length. According to still other embodiments, the ss or dsRNA is 300-600 bases or base pairs in length, 350-500 bases or base pairs in length or 400-450 bases or base pairs in length. In some embodiments, the ss or dsRNA is 400 bases or base pairs in length. It will be appreciated that each individual range of RNA lengths represents a separate and distinct embodiment.

The use of long ss or dsRNAs (i.e. RNA greater than 50 bp) has been very limited owing to the belief that these longer regions of RNA will result in the induction of non specific response. However, the use of long RNAs can provide numerous advantages in that the cell machinery can select the optimal silencing sequence alleviating the need to test numerous siRNAs; long ss or dsRNAs will allow for silencing libraries to have less complexity than would be necessary for siRNAs; and, perhaps most importantly, long ss or dsRNA could prevent viral escape mutations when used as therapeutics.

Various studies demonstrate that long RNAs can be used to silence gene expression without inducing the stress response or causing significant off-target effects—see for example [Strat et al., Nucleic Acids Research, 2006, Vol. 34, No. 13 3803-3810; Bhargava A et al. Brain Res. Protoc. 2004; 13:115-125; Diallo M., et al., Oligonucleotides. 2003; 13:381-392; Paddison P. J., et al., Proc. Natl Acad. Sci. USA. 2002; 99:1443-1448; Tran N., et al., FEBS Lett. 2004; 573:127-134].

In some embodiments, the dsRNA can comprise siRNA duplexes. The term “siRNA” refers to small inhibitory RNA duplexes (generally between 17-30 basepairs, but also longer e.g., 31-50 bp) that induce the RNA interference (RNAi) pathway. Typically, siRNAs are chemically synthesized as 21mers with a central 19 bp duplex region and symmetric 2-base 3′-overhangs on the termini, although it has been recently described that chemically synthesized RNA duplexes of 25-30 base length can have as much as a 100-fold increase in potency compared with 21mers at the same location. The observed increased potency obtained using longer RNAs in triggering RNAi is theorized to result from providing Dicer with a substrate (27mer) instead of a product (21mer) and that this improves the rate or efficiency of entry of the siRNA duplex into RISC.

It has been found that position of the 3′-overhang influences potency of an siRNA and asymmetric duplexes having a 3′-overhang on the antisense strand are generally more potent than those with the 3′-overhang on the sense strand (Rose et al., 2005). This can be attributed to asymmetrical strand loading into RISC, as the opposite efficacy patterns are observed when targeting the antisense transcript.

The strands of a double-stranded interfering RNA (e.g., an siRNA) may be connected to form a hairpin or stem-loop structure (e.g., an shRNA). Thus, as mentioned the dsRNA of some embodiments of the invention may also be a short hairpin RNA (shRNA). A short hairpin RNA (shRNA) can also result from internal stem- and loop base-pairing within a single stranded RNA of the invention.

The term “shRNA”, as used herein, refers to an RNA agent having a stem-loop structure, comprising a first and second region of complementary sequence, the degree of complementarity and orientation of the regions being sufficient such that base pairing occurs between the regions, the first and second regions being joined by a loop region, the loop resulting from a lack of base pairing between nucleotides (or nucleotide analogs) within the loop region. The number of nucleotides in the loop is a number between and including 3 to 23, or 5 to 15, or 7 to 13, or 4 to 9, or 9 to 11. It will be appreciated that each of the ranges noted here represents a separate embodiment of the invention. Some of the nucleotides in the loop can be involved in base-pair interactions with other nucleotides in the loop. Examples of oligonucleotide sequences that can be used to form the loop include 5′-UUCAAGAGA-3′ (SEQ ID NO: 44) (Brummelkamp, T. R. et al. (2002) Science 296: 550) and 5′-UUUGUGUAG-3′ (SEQ ID NO: 45) (Castanotto, D. et al. (2002) RNA 8:1454). It will be recognized by one of skill in the art that the resulting single chain oligonucleotide forms a stem-loop or hairpin structure comprising a double-stranded region capable of interacting with the RNAi machinery.

According to the present teachings, the ss or dsRNA molecules may be naturally occurring or synthetic.

Synthesis of RNA suitable for use with some embodiments of the invention can be affected as follows. First, the target mRNA (e.g. mRNA of a virus pathogenic to farmed crustaceans) sequence is scanned downstream of the AUG start codon for AA dinucleotide sequences. Occurrence of each AA and the 3′ adjacent 19 nucleotides is recorded as potential siRNA target sites. Preferably, siRNA target sites are selected from the open reading frame, as untranslated regions (UTRs) are richer in regulatory protein binding sites. UTR-binding proteins and/or translation initiation complexes may interfere with binding of the siRNA endonuclease complex [Tuschl ChemBiochem. 2:239-245]. It will be appreciated though, that siRNAs directed at untranslated regions may also be effective, as demonstrated for GAPDH wherein siRNA directed at the 5′ UTR mediated about 90% decrease in cellular GAPDH mRNA and completely abolished protein level (see Ambion website—techlib 91/912).

Second, potential target sites are compared to an appropriate genomic database (e.g., crustacean) using any sequence alignment software, such as the BLAST software available from the NCBI server (www(dot)ncbi(dot)nlm(dot)nih(dot)gov/BLAST/). Putative target sites which exhibit significant homology to other coding sequences are identified and filtered out.

Thus, in some embodiments the at least one RNA which is at least partially complementary to the target nucleic acid molecule, or binds to the target molecule through complementary base pairing, or has at least one sequence identical to at least 20 contiguous bases of the target mRNA molecule does not have any significant homology to any of the farmed aquatic crustacean host gene sequences.

Qualifying target sequences are selected as template for siRNA synthesis. Preferred sequences are those including low G/C content as these have proven to be more effective in mediating gene silencing as compared to those with G/C content higher than 55%. Several target sites are preferably selected along the length of the target gene for evaluation. For better evaluation of the selected siRNAs, a negative control is preferably used in conjunction. Negative control siRNA can include the same nucleotide composition as the siRNAs but lack significant homology to the genome. Thus, a scrambled nucleotide sequence of the siRNA is often used, provided it does not display any significant homology to any other gene.

The ss or dsRNA may be synthesized using any method known in the art, including either enzymatic syntheses or solid-phase syntheses. These are especially useful in the case of short polynucleotide sequences with or without modifications as explained above. Equipment and reagents for executing solid-phase synthesis are commercially available from, for example, Applied Biosystems. Any other means for such synthesis may also be employed; the actual synthesis of the oligonucleotides is well within the capabilities of one skilled in the art and can be accomplished via established methodologies as detailed in, for example: Sambrook, J. and Russell, D. W. (2001), “Molecular Cloning: A Laboratory Manual”; Ausubel, R. M. et al., eds. (1994, 1989), “Current Protocols in Molecular Biology,” Volumes I-III, John Wiley & Sons, Baltimore, Maryland; Perbal, B. (1988), “A Practical Guide to Molecular Cloning,” John Wiley & Sons, New York; and Gait, M. J., ed. (1984), “Oligonucleotide Synthesis”; utilizing solid-phase chemistry, e.g. cyanoethyl phosphoramidite followed by deprotection, desalting, and purification by, for example, an automated trityl-on method or HPLC.

The ss or dsRNA can be a mixture of long and short dsRNA molecules such as, dsRNA, siRNA, siRNA+dsRNA, siRNA+miRNA, hpRNA or a combination of same. According to a specific embodiment, the dsRNA is an siRNA (100%). The chitosan-RNA particles can also comprise a mixture of sense and anti-sense single stranded RNA.

It will be appreciated that the ss or dsRNA of some embodiments of the invention need not be limited to those molecules containing only RNA, but further encompasses chemically-modified nucleotides and non-nucleotides.

In some embodiments, the RNA provided herein can be functionally associated with a cell-penetrating peptide. As used herein, a “cell-penetrating peptide” is a peptide that comprises a short (about 12-30 residues) amino acid sequence or functional motif that confers the energy-independent (i.e., non-endocytotic) translocation properties associated with transport of the membrane-permeable complex across the plasma and/or nuclear membranes of a cell. The cell-penetrating peptide used in the membrane-permeable complex of some embodiments of the invention preferably comprises at least one non-functional cysteine residue, which is either free or derivatized to form a disulfide link with a double-stranded ribonucleic acid that has been modified for such linkage. Representative amino acid motifs conferring such properties are listed in U.S. Pat. No. 6,348,185, the contents of which are expressly incorporated herein by reference. The cell-penetrating peptides of some embodiments of the invention preferably include, but are not limited to, penetratin, transportan, pIsl, TAT(48-60), pVEC, MTS, and MAP. On the other hand, in some embodiments, the use of a cell penetrating peptide or transformation agent is undesirable-thus, in some embodiments, the dsRNA is provided lacking, devoid of a cell penetrating peptide or any other form of cell penetrating agents, transformation enhancing agents. It will be noted that, in the context of the present invention, cell penetrating peptide, cell penetrating agents and transformation enhancing agents do not include chitosan or a polymer such as polyethylene glycol (PEG).

According to another embodiment the ss or dsRNA may be a microRNA precursor, or “pri-miRNA”.

The term “microRNA”, “miRNA”, and “miR” are synonymous and refer to a collection of non-coding single-stranded RNA molecules of about 19-28 nucleotides in length, which regulate gene expression. miRNAs are found in a wide range of organisms (viruses.fwdarw.humans) and have been shown to play a role in development, homeostasis, and disease etiology.

Below is a brief description of the mechanism of miRNA activity.

Genes coding for miRNAs are transcribed leading to production of an miRNA precursor “the pri-miRNA”. The pri-miRNA is typically part of a polycistronic RNA comprising multiple pri-miRNAs. The pri-miRNA may form a hairpin with a stem and loop. The stem may comprise mismatched bases.

The hairpin structure of the pri-miRNA is recognized by Drosha, which is an RNase III endonuclease. Drosha typically recognizes terminal loops in the pri-miRNA and cleaves approximately two helical turns into the stem to produce a 60-70 nucleotide precursor known as the pre-miRNA. Drosha cleaves the pri-miRNA with a staggered cut typical of RNase III endonucleases yielding a pre-miRNA stem loop with a 5′ phosphate and ˜2 nucleotide 3′ overhang. It is estimated that approximately one helical turn of stem (˜10 nucleotides) extending beyond the Drosha cleavage site is essential for efficient processing. The pre-miRNA is then actively transported from the nucleus to the cytoplasm by Ran-GTP and the export receptor Ex-portin-5.

The double-stranded stem of the pre-miRNA is then recognized by Dicer, which is also an RNase III endonuclease. Dicer may also recognize the 5′ phosphate and 3′ overhang at the base of the stem loop. Dicer then cleaves off the terminal loop two helical turns away from the base of the stem loop leaving an additional 5′ phosphate and ˜2 nucleotide 3′ overhang. The resulting siRNA-like duplex, which may comprise mismatches, comprises the mature miRNA and a similar-sized fragment known as the miRNA*. The miRNA and miRNA* may be derived from opposing arms of the pri-miRNA and pre-miRNA. MiRNA* sequences may be found in libraries of cloned miRNAs but typically at lower frequency than the miRNAs.

Although initially present as a double-stranded species with miRNA*, the miRNA eventually become incorporated as a single-stranded RNA into a ribonucleoprotein complex known as the RNA-induced silencing complex (RISC). Various proteins can form the RISC, which can lead to variability in specificity for miRNA/miRNA* duplexes, binding site of the target gene, activity of miRNA (repress or activate), and which strand of the miRNA/miRNA* duplex is loaded in to the RISC.

When the miRNA strand of the miRNA:miRNA* duplex is loaded into the RISC, the miRNA* is removed and degraded. The strand of the miRNA:miRNA* duplex that is loaded into the RISC is the strand whose 5′ end is less tightly paired. In cases where both ends of the miRNA:miRNA* have roughly equivalent 5′ pairing, both miRNA and miRNA* may have gene silencing activity.

The RISC identifies target nucleic acids based on high levels of complementarity between the miRNA and the mRNA, especially by nucleotides 2-7 of the miRNA.

A number of studies have looked at the base-pairing requirement between miRNA and its mRNA target for achieving efficient inhibition of translation (reviewed by Bartel 2004, Cell 116-281). In mammalian cells, the first 8 nucleotides of the miRNA may be important (Doench & Sharp 2004 GenesDev 2004-504). However, other parts of the microRNA may also participate in mRNA binding. Moreover, sufficient base pairing at the 3′ can compensate for insufficient pairing at the 5′ (Brennecke et al, 2005 PLoS 3-e85). Computation studies, analyzing miRNA binding on whole genomes have suggested a specific role for bases 2-7 at the 5′ of the miRNA in target binding but the role of the first nucleotide, found usually to be “A” was also recognized (Lewis et at 2005 Cell 120-15). Similarly, nucleotides 1-7 or 2-8 were used to identify and validate targets by Krek et al (2005, Nat Genet 37-495).

The target sites in the mRNA may be in the 5′ UTR, the 3′ UTR or in the coding region. Interestingly, multiple miRNAs may regulate the same mRNA target by recognizing the same or multiple sites. The presence of multiple miRNA binding sites in most genetically identified targets may indicate that the cooperative action of multiple RISCs provides the most efficient translational inhibition.

MiRNAs may direct the RISC to downregulate gene expression by either of two mechanisms: mRNA cleavage or translational repression. The miRNA may specify cleavage off the mRNA if the mRNA has a certain degree of complementarity to the miRNA. When a miRNA guides cleavage, the cut is typically between the nucleotides pairing to residues 10 and 11 of the miRNA. Alternatively, the miRNA may repress translation if the miRNA does not have the requisite degree of complementarity to the miRNA. Translational repression may be more prevalent in animals since animals may have a lower degree of complementarity between the miRNA and binding site.

It should be noted that there may be variability in the 5′ and 3′ ends of any pair of miRNA and miRNA*. This variability may be due to variability in the enzymatic processing of Drosha and Dicer with respect to the site of cleavage. Variability at the 5′ and 3′ ends of miRNA and miRNA* may also be due to mismatches in the stem structures of the pri-miRNA and pre-miRNA. The mismatches of the stem strands may lead to a population of different hairpin structures. Variability in the stem structures may also lead to variability in the products of cleavage by Drosha and Dicer.

Thus, according to some embodiments, the chitosan-RNA particle comprises at least one RNA sequence capable of binding through complementary base pairing to a target mRNA molecule of the virus pathogenic in farmed crustaceans, and particularly, farmed aquatic crustaceans. The target mRNA can be a transcript of any gene sequences of the pathogenic virus. Genes from the pathogenic viruses having sequences complementary to such target mRNA sequences are considered target genes, provided that the target sequences have no substantial homology to the host gene.

In another embodiment, the chitosan-dsRNA particle comprises at least one RNA sequence at least partially complementary to a target mRNA of a virus pathogenic in farmed crustaceans, and particularly, farmed aquatic crustaceans.

In yet another embodiment, the chitosan-dsRNA particle comprises at least one RNA sequence at least 90% identical to a target mRNA of a virus pathogenic in farmed crustaceans, and particularly, farmed aquatic crustaceans.

As used herein, the phrase “pathogen target gene” or “viral target gene” is defined as a viral gene, the expression of which is essential to the virulence, growth or pathogenicity of the virus. The term “gene” is used broadly to refer to any segment of the viral nucleic acid associated with a biological function. Thus, genes include coding sequences and/or the regulatory sequences required for their expression. For example, gene refers to a nucleic acid fragment that expresses mRNA or functional RNA, and optionally encodes a specific protein.

Viral pathogen target genes include, but are not limited to structural genes for virion proteins, genes for functional proteins (e.g. genes associated with replication) and other sequences associated with disease or disease symptoms in the host crustacean.

As used herein, the phrase “viral pathogen gene product” refers to a product of the expression of a viral pathogen gene—including, but not limited to the RNA transcript of the viral pathogen gene and a peptide or polypeptide encoded by a sequence of a viral pathogen gene.

Treating or preventing the viral pathogen infection is achieved by reducing the expression of a viral pathogen gene. Thus, in some embodiments, the at least one viral pathogen gene is a viral gene whose expression is initiated or increased during the course of the viral pathogen infection. For example, Tsai et al (J Virol 204, 78:11360-370) discloses White Spot Syndrome Virus genes, the expression of which can be altered (e.g. decreased), during the viral pathogen infection.

Table I provides a non-limiting list of viral diseases and viral genomes associated with diseases of farmed crustaceans, which comprise sequences which can be targets for reduction in expression by the chitosan-dsRNA particle of the invention.

TABLE I
Diseases and Pathogenic Viruses in Aquaculture

| Host | | | Genome |
|---|---|---|---|
| Organism | Disease | Pathogen (type of organism) | Accession # |
| Crustacean | Infectious pancreatic | IPN virus | NC 001915.1(A) |
| | necrosis (IPN). | | NC 001916.1(B) |
| Crustacean | gill- associated virus | GAV virus | NC_010306.1 |
| | (GAV) | | |
| Crustacean | Infectious Hypodermal | IHHN Virus | NC_002190 |
| | and Haematopoietic | | |
| | Necrosis (IHHN) | | |
| Crustacean | Infectious Myonecrosis | IMN Virus | KR815474.1 |
| | (IMN) | | |
| Crustacean | Infectious haematopoietic | IHNV virus | X89213 |
| | necrosis virus (IHNV) | | |
| Crustacean | Taura Syndrome | Taura Syndrome Virus | NC_003005 |
| Crustacean | White Spot Syndrome | WSS Virus (DNA virus) | AF332093 |
| Crustacean | White Tail Disease (aka | Macrobracium rosenbergii | NC_005094.1(1) |
| | White Muscle Disease) | nodavirus (MrN Virus) | NC-005095.1(2) |
| Crustacean | Yellowhead Disease | YH Virus (ssRNA virus) | FJ848673.1 |
| Crustacean | White Head disease | Decapod Iridescent Virus 1 (DIV1) | MF599468 |

In one embodiment, the at least one viral pathogen gene is selected from the group consisting of coat (or capsid) proteins, enzymes of viral replication (e.g. viral RNA polymerases, helicases), viral proteases and viral chitin-binding proteins. Thus the chitosan-dsRNA particle can comprise at least one RNA sequence complementary to or capable of binding through complementary base pairing to an mRNA sequence of at least one viral coat or capsid, and/or at least one viral enzyme of viral replication, and/or at least one viral protease and/or at least one viral chitin binding protein.

Table II provides a non-exhaustive list of some genes (and their category) of the pathogen viruses of Table I, which can be targets (e.g. targeted gene products) for reduction of expression by the chitosan-dsRNA particle described herein.

TABLE II
PARTIAL LIST OF VIRAL PATHOGEN
GENES FROM CRUSTACEAN VIRUSES

| | GENE | | |
|---|---|---|---|
| Seq ID NO: | SYMBOL | GENE | CATEGORY |
| 1 | DQ681069 | Viral capsid protein | Coat (capsid) |
| | | 28 (VP28) | |
| 2 | DQ681071 | Viral capsid protein | Coat (capsid) |
| | | 19 (VP19) | |
| 3 | AF144620 | Ribonucleotide | Undefined |
| | | reductase2 | |
| 4 | FJ756475 | Thymidine kinase | Undefined |
| | | thymidylate kinase | |
| 5 | DQ121373 | WSV477 | Undefined |
| 6 | AY220746.1 | VP26 | Coat (capsid) |
| 7 | AF510518 | Taura syndrome virus | Coat (capsid) |
| | | coat protein 1 (VP1) | |
| 8 | JQ356865 | Taura syndrome virus | Coat (capsid) |
| | | capsid protein 2 (CP2) | |
| 9 | EU977579 | YHV RNA-dependent | Enzyme of viral |
| | | RNA polymerase | replication |
| 10 | EU977580 | YHV Helicase | Enzyme of viral |
| | | | replication |
| 11 | EU977577 | YHV protease | Viral protease |
| 12 | EU784973 | GAV pplab gene | Enzyme of viral |
| | | | replication |
| 13 | KT316281 | GAV polyprotein pp3 | Coat (capsid) |
| 14 | KF278563 | GAV replicase | Enzyme of viral |
| | | polyprotein 1b | replication |
| 15 | DQ978362 | GAV glycoprotein | Coat (capsid) |
| | | 64 gene | |
| 16 | EU977581 | GAV nucleocapsid gene | Coat (capsid) |
| 17 | EU977578 | GAV YHV-PmA | Enzyme of viral |
| | | replicase | |
| | | polyprotein 1ab | replication |
| 18 | KT316282 | GAV envelope structural | Coat (capsid) |
| 19 | JN616415 | glycoprotein IHHNV | Coat (capsid) |
| | | NS1, IHHNV NS2, | |
| | | Capsid protein | |
| 20 | KJ636783 | Penaeid shrimp | Coat (capsid) and |
| | | infectious myonecrosis | enzymes of viral |
| | | ORF1/ORF2 | replication |
| 21 | D26527 | IPNV RNA-dependent | Enzyme of viral |
| | | RNA polymerase | replication |
| 22 | JX083063 | IPNV Capsid | Coat (capsid) |
| | | protein VP2 | |
| 23 | KC710379 | IPNV VP4 | Viral protease |
| 24 | L40580 | IPNV VP5 | Viral protease |
| 94 | KJ636782.2 | IMNV partial residue | dsRNA binding |
| | | 473-1060 | motif |

It will be appreciated that each gene or sequence disclosed in Table II represents a separate embodiment of the invention.

In some embodiments, the RNA comprises a nucleic acid sequence which specifically reduces the gene products of a gene selected from the group consisting of a viral capsid protein 28 (VP28) gene, a viral capsid protein 19 (VP 19) gene, a ribonucleotide reductase 2 (rr2) gene and a White Spot Virus WSV477 gene. In some embodiments, the WSSV viral capsid protein 28 gene product is encoded by SEQ ID NO: 1, or a portion thereof, the WSSV viral capsid protein 19 gene product is encoded by SEQ ID NO: 2, or a portion thereof, the WSSV ribonucleotide reductase 2 (rr2) protein gene product is encoded by SEQ ID NO: 3, or a portion thereof and the WSV477 gene product is encoded by SEQ ID NO: 5, or a portion thereof. For example, a ss or dsRNA targeted to a viral pathogen of farmed crustaceans can be a WSSV-specific ss or dsRNA corresponding to WSSV sequences SEQ ID NOs: 1 and 2. Additional suitable ss or dsRNA targeted to viral pathogens can be designed according to sequences from any virus (or viruses) pathogenic in farmed crustaceans, for example, the sequences detailed in Table II.

The chitosan-ss or dsRNA particle of the invention can also be used to silence expression of endogenous host genes of the farmed crustaceans. Thus, in some embodiments, the at least one RNA comprises at least one sequence binding to, at least partially complementary to or at least 90% identical to the target mRNA molecule of the farmed crustacean or farmed aquatic crustacean. Endogenous mRNAs of the farmed aquatic crustacean to be targeted using dsRNA can include, but are not limited to, those whose expression is correlated with an undesired phenotypic trait, or those whose expression is associated with susceptibility to, response to or resistance to infection by any of the viruses pathological in farmed crustaceans. Exemplary mRNAs that may be targeted are those encoding viral recognition moieties, whose silencing would not be detrimental to the host crustacean. In other embodiments, the dsRNA is directed to a target mRNA of the farmed aquatic crustacean which constitutes a regulatory step of a desired process or pathway, which silencing thereof increases the activity of the pathway.

Such targets could include mRNA of negative regulators such as transcription factors and signaling pathways, mRNA of receptors responsible for downregulation of desired pathways, etc. Other desirable endogenous targets for inhibition of expression could include, but are not limited to genes and pathways responsible for enhanced growth, enhanced growth rate, improved feed conversion, targeting sex determination during development, influencing flavor, stress (temperature, salinity, toxicity, low Oxygen tension, disease, parasites, etc) resistance or tolerance, breeding behavior and even domestication of species not yet easily cultured. In one specific embodiment, the endogenous host gene target is the shrimp Rab7 gene, encoding a Ras-related viral protein VP-28-binding protein that has been implicated in White Spot Syndrome Virus (WSSV). In some embodiments, the chitosan-RNA particle comprises at least one sequence complementary to or binding with an mRNA sequence of the Litopenaeus vannamei Ras-related protein (Rab7, accession #JQ581679, SEQ ID NO: 25). In further embodiments, the chitosan-dsRNA particle comprises a dsRNA comprising a sequence binding to, at least partially complementary to or at least 90% identical to SEQ ID NO: 47 of the Rab7 mRNA.

Also contemplated are chitosan-RNA particles comprising multiple RNAs. Thus, in some embodiments the dsRNA of the chitosan-dsRNA particle can be homogeneous, i.e. all complementary to, binding to or at least 90% identical to the same sequence of the mRNA target molecule of the viral pathogen. In addition, the ss or dsRNA of the chitosan-RNA particle can be heterogeneous—e.g. complementary to, binding to or at least 90% identical to two or more sequences of the same mRNA target molecule of the viral pathogen. In other embodiments, the ss or dsRNA can comprise sequences complementary to, binding to or at least 90% identical to different mRNA target molecules of the same viral pathogen. For example, in one specific embodiment the RNA of the chitosan-RNA particle described herein comprises sequences complementary to, binding to or at least 90% identical to any one, two, three or more sequences of the WSSV. In some embodiments, the RNA comprises sequences complementary to, binding to or at least 90% identical to any one or more of SEQ ID Nos. 1, 2, 3 and 5. The additional sequences can thus also be complementary to, binding to or at least 90% identical to the same target mRNA, but from different segments thereof, or the additional sequences can be complementary to or binding to the different and distinct target mRNAs, from the same viral pathogen.

Also contemplated are chitosan-RNA particles wherein the dsRNA of the chitosan-RNA particle comprises ss or dsRNA complementary to, binding to or at least 90% identical to one or more each of nucleotide sequences of mRNA target molecules of two or more distinct viral pathogens. For example, in one specific embodiment, the RNA of the chitosan-dsRNA particle described herein comprises sequences complementary to, binding to or at least 90% identical to at least one each of sequences from the WSSV, GAV, INPV, IMNV or any other virus pathogenic in farmed crustaceans. It will be appreciated that the ss or dsRNA of the particle as described herein can include sequences complementary to, binding to or at least 90% identical to all matter of combinations of target mRNAs—e.g. one or more sequences from the same target mRNA along with one or more sequences from one or more different target mRNAs. Also contemplated are chitosan-RNA particles comprising RNA sequences directed towards multiple targets. Such dsRNA directed towards multiple targets includes contiguous dsRNA sequences designed complementary to, binding to or at least 90% identical to more than one target sequence—e.g. all of SEQ ID Nos. 1, 2, 3 of the WSSV comprised on the same ss or dsRNA polynucleotide, which, when processed, can produce siRNAs complementary to, binding to or at least 90% identical to multiple targets of the WSSV. The advantage of such combinations as described herein is, inter alia, in the economy of production and the simplicity of formulation of the chitosan-RNA using the single ss or dsRNA.

Further, also contemplated are chitosan-RNA particles comprising ss or dsRNA complementary to, binding to or at least 90% identical to target mRNA of one or more viral pathogens of more than one species of farmed crustaceans. Thus, the chitosan-dsRNA particles can comprise, for example, dsRNA complementary to, binding to or at least 90% identical to target mRNA of viruses pathological to any one or more of Litopanaeus vannamei, Panaeus monodon, Penaeus japonicus and/or Macrobrachium rosenbergii. Still further, dsRNA may be complementary to, bind to or at least 90% identical to viral pathogens of different classes of farmed crustaceans, for example, ss or dsRNA targeting viruses of crabs as well as viruses of lobsters. Such multiple-targeted chitosan-RNA particles can be particularly effective in an integrated, multitrophic aquaculture system, where more than one crustacean species is farmed together.

Thus, in some embodiments, the RNA comprises a nucleic acid sequence complementary to, binding to or at least 90% identical to a nucleic acid sequence of White Spot Syndrome Virus viral capsid protein 28 (VP28) gene, a viral capsid protein 19 (VP 19) gene, a ribonucleotide reductase 2 (rr2) gene and a White Spot Virus WSV477 gene. In specific embodiments, the RNA targeting WSSV comprises a ss or ds RNA complementary to, binding to or at least 90% identical to a WSSV RNA sequence selected from the group consisting of VP28 sequence SEQ ID NO: 59, VP28 sequence SEQ ID NO: 64 or 65, Rr2 sequence SEQ ID NO: 78 and Wsv477 sequence SEQ ID NO: 79.

In some embodiments, the RNA targeting WSSV comprises a ss or ds RNA complementary to or at least 90% identical to more than one WSSV RNA sequence, for example, to a combination of two or more WSSV sequences (e.g. VP28, VP19, Rr2, Wsv477). In specific embodiments, the RNA targeting WSSV comprises a ss or ds RNA complementary to or at least 90% identical to a portion of a mix of WSSV sequences (for example SEQ ID NO: 66). In a specific embodiment, the RNA targeting WSSV comprises an ss or ds RNA complementary to or at least 90% identical to VP28-VP19 fusion sequence SEQ ID NO: 77.

In yet further embodiments, the chitosan-RNA particle comprises at least one additional ss or dsRNA comprising at least one additional sequence capable of binding through complementary base pairing or at least 90% identical to a target mRNA molecule of the farmed crustaceans. In other embodiments, the chitosan-RNA particle comprises at least one additional ss or dsRNA comprising at least one additional sequence at least partially complementary to or at least 90% identical to a target mRNA molecule of the farmed crustaceans.

According to yet another embodiment of the present invention, synthesis of ss or dsRNA suitable for use with the present invention can be effected according to viral pathogen target sequences known to integrate into the host genome, target sequences suspected associated with resistance to a viral pathogen infection, target sequences representing intergenic regions of the viral pathogen genome and pathogen-specific sequences shown to be critical for pathogen growth and/or replication. It will be appreciated that, in a further embodiment of the present invention, ss or dsRNA targeted to sequences having a conserved homology between different strains of the viral pathogen, or even between diverse viral pathogens, once such sequences are identified, can be effective against more than one strain of the viral pathogen, or even against different viruses.

In yet further embodiments, the chitosan-RNA particle comprises at least one ss or dsRNA comprising at least one sequence capable of binding through complementary base pairing or at least 90% identical to a target mRNA molecule of a farmed crustaceans and at least one ss or dsRNA comprising at least one sequence capable of binding through complementary base pairing to, binding to or at least 90% identical to target mRNA of one or more viral pathogens of one or more species of farmed crustaceans.

It will be appreciated from the description provided herein above, that contacting aquatic crustacean cells with a miRNA may be affected in a number of ways:
1. Transiently transfecting the host cells with the mature double stranded miRNA;
2. Stably, or transiently transfecting the host cells with an expression vector which encodes the mature miRNA;
3. Stably, or transiently transfecting the host cells with an expression vector which encodes the pre-miRNA. The pre-miRNA sequence may comprise from 45-90, 60-80 or 60-70 nucleotides. The sequence of the pre-miRNA may comprise a miRNA and a miRNA* as set forth herein. The sequence of the pre-miRNA may also be that of a pri-miRNA excluding from 0-160 nucleotides from the 5′ and 3′ ends of the pri-miRNA. The sequence of the pre-miRNA may comprise the sequence of SEQ ID NOS: 5-10 or variants thereof;
4. Stably, or transiently transfecting the host cells with an expression vector which encodes the pri-miRNA The pri-miRNA sequence may comprise from 45-30,000, 50-25,000, 100-20,000, 1,000-1,500 or 80-100 nucleotides. The sequence of the pri-miRNA may comprise a pre-miRNA, miRNA and miRNA*, as set forth herein, and variants thereof.

According to some aspects of the invention, there is provided a method for producing a particulate chitosan-RNA composition, comprising mixing the chitosan and intended RNA payload. The present inventors have uncovered that attention to certain aspects of the mixing and processing of the chitosan-RNA composition can result in particulate compositions with advantageous characteristics.

In some embodiments, the mixing of the chitosan and RNA comprises rapidly adding RNA and chitosan solutions while rapidly agitating (e.g. approximately 1500 rpm), and rapidly agitating the mixture for 1-10 minutes (e.g. approximately 1500 rpm). In some embodiments, the mixture is agitated for 1-5 minutes. In other embodiments, the mixture can be agitated for 1, 2, 3, 4, 5, 6, 7, 8, 9, or 10 minutes. In particular embodiments, the chitosan solution comprises 2-20 mg/ml low molecular weight chitosan in mild acidic buffer. In particular embodiments, the chitosan solution is 3 mg/ml. In specific embodiments, the buffer is acidified with an organic acid. In some embodiments, the organic acid is selected from acetic acid, lactic acid, propionic acid and citric acid. In specific embodiments, the chitosan solution is 3 mg/ml low molecular weight chitosan in 0.2 M citric acid buffer, pH 4-5. In other embodiments, the chitosan solution is 3 mg/ml low molecular weight chitosan in 0.02 M citric acid buffer, pH 4-5.

In some embodiments, the RNA solution comprises 1-5 mg/ml RNA in water. In specific embodiments, the RNA and chitosan are mixed at a ratio (RNA:chitosan) in the range of 1:0.14 to 1:1, volume/volume. In specific embodiments, the RNA solution is 3 mg/ml RNA in water, and the RNA:chitosan ratio when mixing is 1:1, volume/volume In still further embodiments, the RNA is provided in a solution of a sodium salt such as sodium chloride, sodium sulfate or sodium citrate. Inasmuch as the presence of chloride ions is less desirable for scaled-up industrial processes, in some embodiments the sodium salt is a non-chloride sodium salt, e.g. sodium citrate or sodium sulfate.

In some embodiments, the pH of the chitosan-RNA complex is pH 3.5-5.0. In other embodiments, the chitosan-RNA particle has a pH of 3.5, 3.6, 3.7, 3.8, 3.9, 4.0, 4.1, 4.2, 4.3, 4.4, 4.5, 4.6, 4.7, 4.8, 4.9 or 5.0.

In some embodiments, following agitation, the mixture is desalted and concentrated. In some embodiments, following desalting and concentration, the chitosan-RNA complex solution is further dried to produce the particulate chitosan-RNA composition. In some some embodiments the chitosan-RNA complex solution is dried using for example, but not exclusively, a spray dryer, a belt dryer, a drum filter, a Nutche filter and the like. Following the drying step, in some embodiments the dried chitodan-RNA composition is ground to approximate the size of shrimp feed particles (micron grade). In some embodiments, the size of the dried particle can be varied, ranging in size from 20 microns to millimeters, according to need (size of the animal to be treated, type of feed, etc). In some embodiments, the dried particle is 20, 30, 40, 50, 60, 70, 100, 120, 140, 160, 180, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000 microns, 1.25, 1.5, 1.75, 2.0, 2.25, 2.5, 2.75, 3.0, 3.5, 4.0, 4.5, 5.0-10 millimeters in size.

Thus, in specific embodiments, there is provided a method for producing a particulate chitosan-RNA composition comprising:
i) rapidly adding an RNA solution to 50-100% deacetylated low molecular weight chitosan solution, wherein said chitosan solution comprises 3 mg/ml low molecular weight chitosan in 0.1-0.01 M organic acid, in a ratio of 1:1 volumes of chitosan to RNA,
(ii) desalting and concentrating the chitosan-RNA complex solution, and
(iii) drying the desalted, concentrated chitosan-RNA complex solution,
thereby producing the particulate chitosan-RNA composition.

Following drying, the particulate chitosan-RNA composition can be used immediately, or stored until use. In some embodiments, the dried particulate chitosan-RNA composition can be stored until mixing with other components to form a pre-mix which can be provided to a feed production facility, for preparation of the feed comprising the particulate chitosan-RNA composition or nutriceutical.

Chemical modifications of chitosan have been pursued to enhance its gene transfection efficiency. Most commonly, chitosan can be structurally modified with hydrophobic, hydrophilic, amphiphilic, CPPs, and cell specific ligands using the reactive amino group (C2 position) and hydroxyl groups (C6 and C3 positions).

Thus, the chitosan-RNA particles of the invention can also be prepared with chemical modifications (in addition to reduction of degree of deacetylation described above), for example, to enhance the effectiveness of binding and delivery of the ss or dsRNA at the target cell. Thus, according to some embodiments, the chitosan-RNA particle further comprises a polymer. A non-limiting list of polymers suitable for addition to the chitosan-RNA particle of the invention includes poly(lysine) (PLL), linear polyethyleneimine (1-PEI), branched polyethyleneimine (b-PEI), poly(ethylene glycol) (PEG), G3 dendritic poly(amido amine) (PAMAM), linear poly(amino amine) (PAA), poly(lactide-co-glycolide) (PLGA) and poly (beta-amino ester) (PBAE).

The ability of the particles to maintain their structural integrity is critical to their efficacy as delivery vehicles for the RNA “payload”. It will be appreciated that complexing of the RNA into the chitosan-RNA particle enhances the stability of the nucleic acid, as shown for particulate compositions of chitosan-dsRNA particles (see Example I, “RNase tolerance”). Thus, in some embodiments, the chitosan-RNA particle is a stable particle.

As used herein, the term “stable chitosan-RNA particle” is defined as a particle which maintains a significant portion of the RNA “payload”, even under difficult conditions. One suitable measure of stability is the ability to maintain a majority of the initial RNA “payload”. Thus, in some embodiments, the chitosan-RNA particle is a stable particle, retaining at least 60% of the RNA of the complex when exposed to an aqueous environment (seawater, freshwater, brackish water, etc). In other embodiments, the chitosan-RNA particle retains at least 60%, at least 70%, at least 80%, at least 90% or more of the RNA of the complex when exposed to an aqueous environment or medium. In specific embodiments, the chitosan-RNA particle retains at least 60%, at least 70%, at least 80%, at least 90% or more of the RNA of the complex when exposed to an aqueous environment at a pH range of 4.2-8.0. In some embodiments, the pH of the aqueous environment or medium is in the range of 6.5-7.8, or, more specifically, pH 7.5.

The particulate compositions and/or chitosan-dsRNA particles as disclosed can be used for treating or preventing disease in farmed crustaceans. The disclosed particles, or compositions comprising the particles can be provided or administered to the farmed crustaceans in a variety of methods of administration, including feeding, contact with gill tissue, via the eyes, via the skin or cuticle (particularly important during a molt) and via external wounds (cuts or abrasions). Parenteral administration of the compositions comprising the particles as disclosed is also contemplated.

In particular embodiments, the disclosed particulate compositions and/or chitosan-RNA particle can be provided (e.g. administered) as a nutriceutical composition, comprising farmed crustacean food and the particulate compositions or chitosan-RNA particles disclosed herein. In some embodiments, the nutriceutical composition comprises particles comprising ss or dsRNA of a single sequence, i.e. all directed to a single target RNA sequence. In other embodiments, the nutriceutical comprises chitosan-RNA particles comprising ss or dsRNA directed to more than one target mRNA sequence, as detailed herein. In all, it will be noted that the nutriceutical can comprise any particulate compositions, chitosan-RNA particle, or any combination thereof described or suggested herein.

As used herein, the term “nutriceutical composition” is a combination of “nutritional composition” and “pharmaceutical composition” and refers to an ingestible substance that has one or more beneficial effects on an organism such as a farmed crustacean. The term “nutriceutical” can also refer to one or more compounds which are present in an ingestible substance. Ingestible substances include, but not limited to dietary supplements, foods and the like. The terms “nutriceutical” and “nutritional supplement” may be used interchangeably. A substance (e.g., a food product, a nutriceutical product or a pharmaceutical) having beneficial biological, nutriceutical or medicinal properties refers to the ability of the substance to provide an individual one or more health benefits as described herein (e.g., in the resistance to, prevention, reduction and/or cure of one or more diseases and/or disorders described herein).

The nutriceutical composition can be administered to treat a disease or silence a gene, and examples of farmed crustaceans to be treated and diseases that can be prevented or cured in the treated crustaceans are described herein. Depending on the farmed crustacean to be treated the formulations can be administered either by providing the chitosan-RNA particles with the feed of the crustacean or, in the case of some shrimp or other crustaceans, by providing it in the water in which the animal lives. It will be appreciated that chitosan is derived from chitin, which constitutes a significant portion of the diet of many marine and fresh water species. Yet further, marine crustaceans are known to be cannibalistic, feeding off members of their own communities as well as non-related populations. Thus, the chitosan-RNA particles described herein may also be effective in delivering the ss or dsRNA to the farmed crustacean without combination with feed, provided in the water of the aquaculture environment in which the organism lives.

It will be appreciated that the therapeutic effects of the nutriceutical, when provided as feed, or along with feed in the water of a farmed crustacean subject, may be dependent upon ingestion by the crustacean subject, but may also be mediated by contact with the tissues of the subject crustacean, for example, contact with the gills, eyes or other surfaces which may allow transfer of the particulate compositions or particles described herein, e.g. whole particles, or even just the ss or dsRNA component of the particles to cells of the host crustacean subject.

The particles may be introduced into a crustacean, with a physiologically acceptable vehicle and/or adjuvant. Useful vehicles are well known in the art, and include, e.g., water, buffered water, saline, glycine, hyaluronic acid and the like. The resulting aqueous solutions may be packaged for use as is, or lyophilized, the lyophilized preparation being rehydrated prior to administration, as mentioned above. The compositions may contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, and the like. In one embodiment of the invention, the particle is encapsulated such that the resulting composition is water resistant but amenable to release within the digestive system of the crustacean host. In another embodiment, the molecule is combined with a binder that assists in associating the molecule with feed, which is particularly useful for oral administration. Such a water resistant binding substance can be any substance having such properties. Examples include, without limitation, agarose or other sugar compounds, albumin, alginate or any similar composition.

The compositions of the invention can be provided in feed. Feeds for farmed aquatic species include fish oils and proteins as well as plant proteins, minerals, and vitamins that achieve the nutrition requirements of the fish or crustaceans and may also offer health benefits to humans (consuming the farmed species). Traditionally, diets for fish contained 30-50% fish meal and oil. Feed suitable for feeding farmed aquatic fish, crustaceans and mollusks can be similar or different in formulation. For example, fish can be fed various foodstuffs including but not limited to fishmeal, pea seed meal, wheat bran, wheat flour, blood meal, vitamins, corn gluten and wheat gluten.

Crustaceans can be fed various foodstuffs including but not limited to soy bean oil cake, fish meal, fish oil, wheat flour, soybean meal, squid oil, Brewer's yeast, shrimp meal, squid meal, alfalfa, wheat gluten, squid liver powder, yeast, shrimp head meal, shrimp shell meal and vitamins. These can be provided in many forms, for example, as feed pellets.

The composition of some embodiments of the invention may further comprise at least one of a surface-active agent, an inert carrier vehicle, a preservative, a feeding stimulant, an attractant, an encapsulating agent, a binder, an emulsifier, a dye, an ultra-violet protector, a buffer, a flow agent or micronutrient donors (e.g. vitamins), a drug or other preparations that influence the health or growth, well being or stress/disease tolerance of the farmed crustacean.

The particulate compositions and/or chitosan-RNA particles of the invention can be prepared as a nutriceutical for farmed crustaceans by mixing the particles with feed of the farmed crustaceans. The particulate compositions and/or chitosan-RNA particles of the invention can be mixed with the farmed crustacean food in different ways, for example, the particles can be added to the shrimp feed by mixing the formulation with readymade feed and air drying, then spraying the mixture with fish oil or gelatin. In some embodiments, the particulate compositions and/or chitosan-RNA particles can be mixed with the feed as a formulation (liquid) and then the feed top sprayed with oil or gelatin. The feed can also be top-sprayed with both gelatin and the liquid formulation of chitosan-RNA particles. Dry powder formulations of the particulate compositions or chitosan-RNA particles can also be used, for example, by spray drying the chitosan-RNA particles, mixing the spray dried chitosan-RNA with the feed ingredients and top spraying with oil or gelatin.

In some embodiments, the particles are mixed with the feed at a ratio of about 0.5-10% particles (w/w) in the feed. In some embodiments, the particles are mixed with feed at 0.75-7.5%, 1.0-5.0%, 1.5-4.5%, 2-8%, 1%, 2%, 3%, 4%, 5% or 6%. The particles can also be provided according to dosage of “payload” RNA per gram crustacean feed, for example, 210-10,000 μg ss or dsRNA per gram of shrimp and/or prawn feed, 50-8,000 μg ss or dsRNA per gram of shrimp and/or prawn feed, 100-5,000 μg ss or dsRNA per gram of shrimp and/or prawn feed, 200-1000 μg ss or dsRNA per gram of shrimp and/or prawn feed, 250-5,000 μg ss or dsRNA per gram of shrimp and/or prawn feed, and 500-5,000 μg ss or dsRNA per gram of shrimp and/or prawn feed. It will be appreciated that each individual range of particles or dsRNA per gram feed ratio represents a single, separate embodiment. In a specific embodiment, the particles are mixed with the feed at a ratio of 0.1 to 50 mg ss or dsRNA per gram of shrimp and/or prawn feed. In particular embodiments, the particles or particulate composition are mixed with the feed at a ratio of about 0.1 mg/g feed, 0.5 mg/g feed, 1.0 mg/g feed, 2.0 mg/g feed, 4.0 mg/g feed, 5.0 mg/g feed, 7.5 mg/g feed, 8 mg/g feed, 10 mg/g feed, 12 mg/g feed, 15 mg/g feed, 17.5 mg/g feed, 20 mg/g feed, 22 mg/g feed, 25 mg/g feed, 27.5 mg/g feed, 28 mg/g feed, 30 mg/g feed, 32 mg/g feed, 34 mg/g feed, 36 mg/g feed, 40 mg/g feed, 42 mg/g feed, 44 mg/g feed, 46 mg/g feed, 48 mg/g feed or 50 mg/g feed.

In some embodiments, the nutriceutical composition is defined according to the RNA content (w/w) of the composition. It will be appreciated that the RNA content of the nutriceutical relates to the % of the “payload” ds or ss RNA contributed by the chitosan:RNA particles, and does not include RNA contributed by the feed or any other component of the nutriceutical.

In particular embodiments, the RNA content of the nutriceutical composition is in the range of 0.01-10% RNA (w/w). In other embodiments, the RNA content is in the range of 0.05-5.0%, 0.1-3.0%, 0.25-2.5%, 0.5-2%, 0.1-0.5% (w/w) or intermediate ranges. In specific embodiments, the RNA (w/w) content of the nutriceutical is 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1.0, 1.25, 1.5, 1.75, 2.0 or 2.5%. In yet further embodiments the RNA content (w/w) of the nutriceutical composition is in the range of 0.1-0.5%.

In further embodiments, provision of the particle or nutriceutical composition may be varied throughout the duration of culturing of the crustacean, or during portions thereof. For example, in some embodiments, the particles or particulate composition is provided in the feed for 7 days at 50 mg ss or ds RNA per gram feed, or for 21 days at 0.1 to 1 mg ss or dsRNA per gram feed. It will be understood that regimens can vary over the life of the farmed crustacean, and that various RNA/feed ratios can be implemented at different times and for a variety of durations during the culturing of the farmed crustaceans. Likewise, in some embodiments, administration of the articulate composition or nutraceutical can be initiated upon detection of infection or risk of infection, and in other embodiments, administration can be prophylactic, anticipating and attempting to prevent or attenuate infection.

In some embodiments, wherein the crustacean is shrimp and/or prawns, the particulate composition and/or nutriceutical composition is provided in a dosage of 3-10% of the shrimp's body weight per daily feeding. In specific embodiments the particle or nutriceutical composition of the invention is provided in a dosage of 5% of the shrimp's body weight per feeding. It will be appreciated that the amount of nutriceutical composition provided to the shrimp can vary with the shrimp's body weight. For example, in some embodiments, younger shrimps (e.g. <0.1 g body weight), are fed up to 44% of the shrimp's body weight. In more mature shrimp (e.g. >25 g body weight), for example, feed is provided up to 2% of their body weight.

In other embodiments, some younger shrimps (e.g. <0.50 g body weight), are fed up to 400% of the shrimp's body weight. In more mature shrimp (e.g. >7 g body weight), for example, feed is provided up to 7% of their body weight.

Thus, in particular embodiments, young shrimp, having body mass (weight) between 3-30 milligrams are provided with feed (e.g. the nutriceutical) equaling 90-400% of the body mass per day. In some embodiments, young shrimp, having body mass (weight) between 3-30 milligrams are provided with feed (e.g. the nutriceutical) equaling 90, 100, 120, 140, 160, 180, 200, 220, 240, 260, 280, 300, 320, 340, 360, 380 or 400% of the body mass per day.

In particular embodiments, shrimp having body mass (weight) between 30-milligrams to 3 grams are provided with feed (e.g. the nutriceutical) equaling 7-90% of the body mass per day. In some embodiments shrimp, having body mass (weight) between 30 milligrams and 3 grams are provided with feed (e.g. the nutriceutical) equaling 7, 10, 15, 20, 25, 30, 35, 40, 45, 50, 55, 60, 65, 70, 75, 85 or 90% of the body mass per day.

In still other embodiments, grown shrimp having body mass (weight) between 3-7 grams or greater are provided with feed (e.g. the nutriceutical) equaling 3-7% of the body mass per day. In some embodiments shrimp, having body mass (weight) between 3-7 grams (or greater) are provided with feed (e.g. the nutriceutical) equaling 3, 3.5, 4, 4.5, 5, 5.5, 6, 6.5 or 7% of the body mass per day.

Frequency of provision of the particle or nutriceutical composition (regimen) will vary with the farmed aquatic species, and, in some cases, with the specific stages of the life cycle of the farmed organisms. Thus, in some embodiments, the farmed organisms is shrimp and/or prawn, and the particle or nutriceutical of the invention is provided in 1-10, 2-8, 3-6 or 1-3 feedings per life cycle of the shrimp and/or prawns. In a particular embodiment, the farmed crustacean is shrimp and/or prawns and the particle or nutriceutical of the invention is provided in 1-3 feeding per life cycle of the shrimp and/or prawns. In another embodiment, each of the feedings is at least once, twice, at least three times, at least four, five or more times per day, and each feeding period extends for at least two, at least three, at least four, five or more days. In some particular embodiments, each of the feedings is at least three times per day. In another embodiment, each feeding period extends for at least five days.

The invention therefore provides methods of treating or preventing a disease or silencing a gene by administering to a farmed crustacean the particulate composition, formulation or nutriceutical composition of the invention. The particulate composition, formulation or nutriceutical composition can be administered by any manner described herein. The particulate compositions, particles or nutriceutical of the invention are also useful in the manufacture of a medicament for the treatment of viral diseases or for silencing viral genes, as described herein. A non-limiting listing of viral diseases or conditions of farmed crustaceans suitable for treatment with the compositions and methods of the invention, and the pathogenic viruses associated therewith, is provided herein (see Table 1).

In some embodiments, ingestion of the particulate compositions, particle or nutriceutical composition of the invention by the farmed crustacean results in reduction in the level of the at least one gene product of the pathogenic virus in the farmed crustaceans, compared to the level of the pathogenic virus gene product in at least one of the same farmed crustaceans ingesting feed devoid of the ss or dsRNA targeted to the gene product of the pathogenic virus. In other embodiments, ingestion of the particulate composition, formulation or nutriceutical composition of the invention by the farmed crustacean results in reduction in the level of the pathogenic virus in the farmed crustaceans, compared to the level of the pathogenic organisms in at least one of the same farmed crustaceans ingesting feed devoid of the ss or dsRNA targeted to the gene product of the pathogenic virus.

With the particulate composition, formulation or nutriceutical compositions of the present invention, it is possible to achieve protection against viral disease in a farmed crustacean for long periods. Protection periods after at least one feeding with the particles or nutriceutical composition targeting the pathogenic virus have been achieved and protection of at least two weeks, at least 20 days, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 days or more, can been achieved using the invention. It will be appreciated that each individual period represents a single, separate embodiment.

In some embodiments, ingestion by, or contact with the farmed crustacean, of the particulate composition, formulation or nutriceutical composition results in increased survival, yield, growth rate, vigor, biomass, feed conversion, size, quality of taste and odor or stress tolerance of said farmed crustaceans, compared to said farmed crustaceans ingesting feed devoid of ss or dsRNA targeted to a gene product of said pathologenic virus.

In US US 20190175518 to Ufaz et al, the inventors have shown that administration of chitosan-ssRNA particles as well as chitosan-dsRNA particles comprising RNA sequences targeted to shrimp WSSV viral sequences, are effective in protecting shrimp against infection and the pathologies of lethal WSSV infection. The particulate composition or nutriceutical composition of the present invention have also been shown to be highly effective delivery vehicles for administering gene silencing RNA sequences to aquatic crustaceans. Thus, in some embodiments, ingestion by, or contact with the farmed crustacean, of the chitosan-RNA particulate composition or nutriceutical of the present invention results in increased survival of said farmed crustaceans, compared to said farmed crustaceans ingesting feed devoid of, or not coming in contact with ss or dsRNA targeted to a gene product of said pathologenic virus. As used herein, the term “survival” is defined as maintenance of viability. Lack of “survival” is indicated by death.

As used herein, the terms “viral pathology” or “pathogen viral infection” is defined as undesirable changes in the physiology, morphology, reproductive fitness, economic value, vigor, biomass, taste quality, odor, stress-tolerance of a farmed crustacean, directly or indirectly resulting from contact with a farmed crustacean pathogenic virus. According to one embodiment of the invention, the undesirable changes include, but are not limited to biomass and/or yield of the diseased or pathogen infected crustacean. According to another embodiment of the invention, change in yield includes, but is not limited to change in yield per volume of aquaculture, change in quality of the farmed crustaceans, taste quality and odor quality (e.g. of the flesh) and the like. In some embodiments, the host crustacean is a shrimp, prawn or crayfish and the pathogenic virus is White Spot Syndrome Virus. In some embodiments, the WSSV infection causes White Spot disease in the host crustacean.

Clinical signs of WSSV include a sudden reduction in food consumption, lethargy, loose cuticle and often reddish discolouration, and the presence of white spots of 0.5 to 2.0 mm in diameter on the inside surface of the carapace, appendages and cuticle over the abdominal segments. Histological changes are seen in the gill epithelium, antennal gland, haematopoietic tissue, nervous tissue, connective tissue and intestinal epithelial tissue. Infected cells have prominent intranuclear occlusions that initially stain eosinophilic, but become basophilic with age; hypertrophied nuclei with chromatin margination; and cytoplasmic clearing. Pathogenesis involves widespread tissue necrosis and disintegration.

White spots on the shell of infected shrimp under scanning electron microscope appear as large, dome-shaped spots on the carapace measuring 0.3 to 3 mm in diameter. Smaller white spots of 0.02 to 0.1 mm appear as linked spheres on the cuticle surface. Chemical composition of the spots is similar to the carapace, calcium forming 80-90% of the total material and it is suggested to have derived from abnormalities of the cuticular epidermis.

A number of biochemical changes have been reported after infection with this virus: glucose consumption and plasma lactate concentration increase, glucose 6 phosphate dehydrogenase activity increases and triglyceride concentration decreases. The voltage dependent anion channel of the mitochondrion is also up regulated.

As used herein, the phrase “stress tolerance” refers to both tolerance to biotic stress, and tolerance to abiotic stress. The phrase “abiotic stress” as used herein refers to any adverse effect on metabolism, growth, viability and/or reproduction of a host crustacean caused by a-biotic agents. Abiotic stress can be induced by any of suboptimal environmental growth conditions such as, for example, poor oxygenation, high concentrations of toxins, pollutants or waste in the water, low or high water temperature, heavy metal toxicity, high or low nutrient levels (e.g. nutrient deficiency), high or low salt levels (e.g. salinity), atmospheric pollution, high or low light intensities (e.g. insufficient light) or UV irradiation. Abiotic stress may be a short term effect (e.g. acute effect, e.g. lasting for about a week) or alternatively may be persistent (e.g. chronic effect, e.g. lasting for example 10 days or more). The present disclosure contemplates situations in which there is a single abiotic stress condition or alternatively situations in which two or more abiotic stresses occur.

As used herein the phrase “abiotic stress tolerance” refers to the ability of a farmed aquatic crustacean to endure an abiotic stress without exhibiting substantial physiological or physical damage (e.g. alteration in metabolism, growth, viability and/or reproducibility of the crustacean).

According to some embodiments, ingestion or contact with the particle or nutriceutical of the invention increases growth rate and feed conversion. Growth rate can be measured by biomass or yield, and can be used to feed conversion. As used herein, the phrase “feed conversion” refers to a measure of farmed crustacean production per unit of nutrient provided. Feed conversion efficiency is typically a result of an alteration in at least one of the uptake, spread, absorbance, accumulation, relocation (within the crustacean) and use of nutrients absorbed by the crustacean.

As used herein the term/phrase “biomass”, biomass of an aquatic farmed species or “host crustacean biomass” refers to the amount (e.g., measured in grams of air-dried or wet tissue) of a tissue produced from the host organism in a growing season.

As used herein the term/phrase “vigor” refers to the amount (e.g., measured by weight) of tissue produced by the farmed aquatic crustacean in a given time. Increased vigor could determine or affect the yield or the yield per growing time or growing area.

As used herein the term/phrase “yield” refers to the amount (e.g., as determined by weight or size) or quantity (e.g., numbers) of tissues or organs produced per individual farmed crustacean, a population of the farmed aquatic crustacean or per growing season. Increased yield can affect the economic benefit one can obtain from the aquaculture in a certain growing area and/or growing time.

According to one embodiment the yield is measured by protein content.

As used herein “biotic stress” refers stress that occurs as a result of damage done to the farmed crustaceans by other living organisms, such as bacteria, viruses, fungi, parasites, beneficial and harmful insects.

In some embodiments of the invention, ingestion of or contact with the nutriceutical or particular composition of the invention results in: improved tolerance of abiotic stress (e.g., tolerance of poor water quality, heat, cold, non-optimal nutrient or salt levels) or of biotic stress (e.g., crowding or wounding); a modified primary metabolite (e.g., fatty acid, oil, omega-3 oils, amino acid, protein, sugar, or carbohydrate) composition; a modified secondary metabolite (e.g., alkaloids, terpenoids, polyketides, non-ribosomal peptides, and secondary metabolites of mixed biosynthetic origin) composition; a modified trace element (e.g., iron, zinc), or vitamin (e.g., tocopherols) composition; improved yield (e.g., improved yield under non-stress conditions or improved yield under biotic or abiotic stress); improved ability to use nutrients; modified growth or reproductive characteristics; improved harvest, storage, or processing quality (e.g., improved harvest, storage, or processing quality), improved appeal to consumers; or any combination of these traits.

As used herein the term “improving” or “increasing” refers to at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90% or greater increase in any of the abovementioned parameters, as compared to the same or similar farmed crustaceans infected with the same pathogen or having the same disease, and not ingesting or contacting the nutriceutical or particulate composition (i.e., farmed aquatic crustacean not contacted with or ingesting the nutriceutical or particulate composition) of the disclosure.

As used herein the term “decreasing” refers to at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90% or greater decrease in disease or pathology signs such as white spots, necrosis and the like of the farmed crustacean, as compared to the same or similar untreated crustacean.

In some embodiments, the changes in the negative or positive parameters of the treated farmed crustaceans is measured at a time point 2-3 weeks post treatment, 3-4 weeks post treatment, 5-7 weeks post treatment, 1-2 months post treatment, 2-4 months post treatment, 4-6 months post treatment, 5-8 months post treatment and 5-12 months post treatment or more.

According to some embodiments of the invention, health/disease parameters are monitored in the treated crustaceans following ingestion or contact with the nutriceutical or particle. In some embodiments, monitoring of the parameters (of gene expression and/or tolerance to stress, growth rate, etc) can be used to determine regimen of treatment of the crustaceans, for example, additional introduction of the nutriceutical or particulate composition of the invention, augmentation of the treatment with other treatment modalities (e.g. pestcticide, antibiotics, additional species for multi-species aquaculture, etc). Selection of individuals for monitoring in a tank or pen can be random or systematic (for example, sentinel crustaceans can be pre-selected prior to the treatment).

Also provided by the present invention are farmed crustaceans comprising the particulate composition or the nutriceutical composition described herein. Any farmed crustaceans (e.g. shrimp or prawns) can comprise the particulate composition or nutriceutical composition, and in particular, any one of the group consisting of Shrimp, Prawns, Crabs, Lobsters and Crayfishes. In some specific embodiments the farmed crustacean is a shrimp or prawn. Suitable shrimps or prawns can be selected from the group consisting of Litopanaeus vannamei, Panaeus monodon, Penaeus japonicas and Macrobrachium rosenbergii.

Yet further, in some embodiments there is provided an aquaculture environment comprising the farmed crustacean comprising the particulate compositions, nutriceutical compositions or particles described herein. Such an aquaculture environment can be a pond, a pen or a tank. The pond, tank or pen can further be an open or a closed system aquaculture environment.

Also provided by the present invention are farmed fish or crustaceans comprising the chitosan-RNA particle or the nutriceutical composition described herein. Any farmed fish or crustaceans (e.g. shrimp or prawns) can comprise the particle or nutriceutical composition, and in particular, any one of the group consisting of Shrimp, Prawns, Crabs, Lobsters and Crayfishes. In some specific embodiments the farmed crustacean is a shrimp or prawn. Suitable shrimps or prawns can be selected from the group consisting of Litopanaeus vannamei, Panaeus monodon, Penaeus japonicas and Macrobrachium rosenbergii.

Yet further, in some embodiments there is provided an aquaculture environment comprising the farmed fish or crustacean comprising the particles or nutriceutical compositions described herein.

The term “farmed crustacean” or “farmed fish” as used herein refers to a fish or crustacean which is either strictly or partially aquatic (i.e. living at least a portion of the organism's life cycle in water), and which is cultivated (i.e. grown) by man, in an aquaculture environment. Aquaculture environments can include man-made tanks, man-made ponds, cages, pens and other types of enclosures. The term “aquaculture environment” as used here is synonymous with the term “aquaculture system”. The following describes aspects and different types of aquaculture environments suitable for use with the compositions and methods disclosed herein.

One type of aquaculture environment is mariculture, a branch of aquaculture that cultivates marine organisms either in the open ocean, an enclosed portion of the ocean, or tanks or ponds filled with seawater. Finfish (e.g. flounder and whiting), marine crustaceans (e.g. prawns, shrimp, lobsters and crabs) and marine mollusks (e.g. oysters and abalone) can be cultured in seawater. Fresh water aquaculture is suitable for fresh water species, including fish (e.g. tilapia, trout), crustaceans (e.g. crayfish) and fresh water mollusks (e.g. clams). Some species are particularly suited for culture in brackish water (carp, catfish).

Some aquaculture systems employ running water, to provide oxygenated water and eliminate waste products. Other aquaculture environments, without access to affordable running water recycle part, or all of the water of the enclosures, while still others employ various techniques (spraying, paddles) to aerate the standing water of the enclosure. Some aquaculture systems use water treatment systems such as remediation (e.g. bio-remediation) systems, for example, the re-circulating system described in U.S. Pat. No. 8,506,881 to Bradley, et al.

Aquaculture environments can include open pond systems, such as ponds, pools, pens and lakes, where the water surface is exposed to the open air, or closed-pond systems, in which the pond is covered, allowing better control of sunlight, temperature and gases.

Some aquaculture systems are monoculture systems—a single species of fish or crustacean is farmed within the entire aquaculture environment. Monoculture has advantages (ease of harvest, simple nutritional requirements), but also disadvantages, as farming of a single species does not allow recycling of nutrients. Integrated multitrophic aquaculture (IMTA), on the other hand, cultivates a number of different species together within the same aquaculture environment, and so the nutritional needs of the component species can be combined into the single system which recycles nutrients (e.g. fish and crustacean waste provides nutrients for mollusks and seaweed, when cultivated together).

In some embodiments, the farmed crustaceans suitable for use with the methods and compositions of the present invention are aquatic shrimps, prawns and the like. In some embodiments, farmed crustaceans suitable for use with the invention disclosed herein are selected from the group consisting of Shrimp, Prawns, Crabs, Lobsters and Crayfishes. In particular, in some embodiments the farmed crustaceans are shrimps and/or prawns. Specifically, in some embodiments, the shrimp or prawns are selected from the group consisting of Litopanaeus vannamei, Panaeus monodon, Penaeus japonicus and Macrobrachium rosenbergii.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is understood that any Sequence Identification Number (SEQ ID NO) disclosed in the instant application can refer to either a DNA sequence or a RNA sequence, depending on the context where that SEQ ID NO is mentioned, even if that SEQ ID NO is expressed only in a DNA sequence format or a RNA sequence format. For example, SEQ ID NO: 47 (Rab7) is expressed in a DNA sequence format (e.g., reciting T for thymine), but it can refer to either a DNA sequence that corresponds to a Rab7 nucleic acid sequence, or the RNA sequence of an RNA molecule nucleic acid sequence. Similarly, though some sequences are expressed in a RNA sequence format (e.g., reciting U for uracil), depending on the actual type of molecule being described, it can refer to either the sequence of a RNA molecule comprising a dsRNA, or the sequence of a DNA molecule that corresponds to the RNA sequence shown. In any event, both DNA and RNA molecules having the sequences disclosed with any substitutes are envisioned.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

### Experimental Procedures

Production of dsRNA Extract

The Rab7-dsRNA was synthesized by an in-vivo bacterial system as described previously (see US20190175518 to Ufaz et al). Briefly, for cloning a recombinant plasmid containing an inverted repeat of stem and loop a synthetic fragment containing Litopenaeus vannamei Ras-related protein (Rab7, accession #JQ581679) partial sequence and non-coding addition for the loop (SEQ ID NO: 48, loop) was synthesized (SEQ ID NO: 46, Rab7 fragment with loop).

This fragment was used as template for amplification of sense 250 bp Rab7 sequence+loop region using the primers:

XbaI Rab7 Forward GAAACTCTAGATGGGTAACAAGATTGATCTGGAG (SEQ ID NO: 26) and BamHI Rab7 Reverse AGCCGGATCCtagcttacga (SEQ ID NO: 27). The PCR product was cloned into pET9a (Novagen) using XbaI+BamHI restriction sites. The antisense Rab7 was PCR amplified using the same template with the following primers: BamHI Forward GCATAGGATCCTGGGTAACAAGATTGATCTGGAG (SEQ ID NO: 28) and PstI reverse GAGTACTGCAGCATCCTGTTTAGCCTTGTTGTCA (SEQ ID NO: 29) and was cloned using BamHI, PstI to generate the final plasmid—pET9a-Rab7 250 RNAi. The recombinant plasmid (pET9a-Rab7 250) containing an inverted repeat of stem loop Rab7 was transformed by heat shock method into the ribonuclease III (Rnase III) mutant E. coli strain HT115. Expression of stem loop Rab7 was induced by adding 0.4 mM Isopropyl-β-D Thiogalactoside (IPTG) into the bacterial culture. The culture was harvested 4 hours after IPTG induction. Bacterial single-stranded RNA (ssRNA) and loop region of stem loop Rab7 were digested with ribonuclease A (Rnase A). Then, the dsRNA-GFP was extracted by TRIzol® RNA Isolation Reagents (ThermoFisher Scientific) according to the manufacturer's instructions. Concentration of dsRNA-Rab7 and dsRNA-GFP were determined by UV-spectrophotometry at wavelength 260 nm and agarose gel electrophoresis.

The same method was used for cloning Rab7-RNAi sequences of 125 bp and 70 bp. The primers appear in the table below:

TABLE III
Primer sequences for cloning Rab7-RNAi

| | | RNAi | SEQ |
|---|---|---|---|
| | | seq. | ID |
| Primer name | Sequence | length | NO: |
| XbaI for Rab7 70 | GATCTCTAGAGAGGTGGAGCTGTACAATGAG | 70 | 30 |
| PstI For loop | CGTCTGCAGCGCATCTTGC | 70 | 31 |
| BamHI For Rab7 | GATCGGATCCGAGGTGGAGCTGTACAATGAG | 70 | 32 |
| 70 | | | |
| PstI Rab7 Rev | GACTACTGCAGCATCCTGTTTAGCCTTGTTGTC | 70 | 33 |
| BamHI Rab7 125 | GAATCGGATCCTAATGTGGAGCTAGCTTTCCAG | 125 | 34 |
| PstI rab7s Rev | GAGTACTGCAGCATCCTGTTTAGCCTTGTTGTCA | 125 | 35 |
| PstI loop Rev | tacgtCTGCAGCGCATCTTG | 125 | 36 |
| XbaI rab7 125 | GAATCTCTAGATAATGTGGAGCTAGCTTTCCAG | 125 | 37 |
| For | | | |

The same method was used for cloning VP28-RNAi sequences of 250 bp.

A synthetic DNA sequence encoding the VP28-RNAi polypeptide:

(SEQ ID NO: 82)
CATTCAAGGTGTGGAACAACACATCAAGAAAGATCAACATCACTGGTATG
CAGATGGTGCCAAAGATTAACCCATCAAAGGCCTTTGTCGGTAGCTCCAA
CACCTCCTCCTTCACCCCCGTCTCTATTGATGAGGATGAAGTTGGCACCT
TTGTGTGTGGTACCACCTTTGGCGCACCAATTGCAGCTACCGCCGGTGGA
AATCTTTTCGACATGTACGTGCACGTCACCTACTCTGGCACTGAGACCGA
G

was used for amplification of the sense and antisense fragments using the following primers:

(SEQ ID NO: 83)
XbaI VP28-3 For GACTATCTAGACATTCAAGGTGTGGAACAACAC,
and
(SEQ ID NO: 84)
NcoI VP28-3 Rev ACGTACCATGGCTCGGTCTCAGTGCCAGAGT,
and
(SEQ ID NO: 85)
BamHI VP28-3 For ACATAGGATCCCATTCAAGGTGTGGAACAACAC
and
(SEQ ID NO: 86)
PstI VP28-3 Rev ACAGACTGCAGCTCGGTCTCAGTGCCAGAGT
for the complementary strand.

The amplified sense fragment sense was cloned into a pET9a-rab7 125 RNAi—a recombinant plasmid containing an inverted repeat of stem loop Rab7 using XbaI and NcoI, replacing the Rab7 sequence with the VP28 fragment. The resulting plasmid was then digested with BamHI and PstI and the complementary sequence was cloned using the same enzyme, resulting in a recombinant plasmid (pET9a-VP28 250) containing an inverted repeat of stem loop VP28 250 bp fragment.

The sequence of the complete VP28 RNAi fragment
(Stem and loop) is set forth in SEQ ID NO: 87)
GGATCCCATTCAAGGTGTGGAACAACACATCAAGAAAGATCAACATCACTGGTATG
CAGATGGTGCCAAAGATTAACCCATCAAAGGCCTTTGTCGGTAGCTCCAACACCTCC
TCCTTCACCCCCGTCTCTATTGATGAGGATGAAGTTGGCACCTTTGTGTGTGGTACCA
CCTTTGGCGCACCAATTGCAGCTACCGCCGGTGGAAATCTTTTCGACATGTACGTGC
ACGTCACCTACTCTGGCACTGAGACCGAGCTGCAGCGCATCTTGCGGTTCTTGCACG
AGCCTACTCACGGTATAAATCGAGCGCGCTCCACCCAGACTCATCCATCGTAGCTCC
TAATGCGGATTCAACCTGGCGTTTATATCCGGGAAAGGTGCCTAGGAGTGATAGACT
CTTAAGTCTTTGGGCCATGGCTCGGTCTCAGTGCCAGAGTAGGTGACGTGCACGTAC
ATGTCGAAAAGATTTCCACCGGCGGTAGCTGCAATTGGTGCGCCAAAGGTGGTACC
ACACACAAAGGTGCCAACTTCATCCTCATCAATAGAGACGGGGGTGAAGGAGGAGG
TGTTGGAGCTACCGACAAAGGCCTTTGATGGGTTAATCTTTGGCACCATCTGCATAC
CAGTGATGTTGATCTTTCTTGATGTGTTGTTCCACACCTTGAATGTCTAGA

For dsRNA recovery—dsRNA was recovered from E. coli cells following fermentation process. A bacterial suspension was prepared by adding lysis buffer containing 50 mM phosphate buffer+0.3M NaCl to the E. coli pellet. The bacterial suspension was then homogenized using a high pressure homogenizer at 800 atm. The resulting cell lysate was adjusted to pH 4 using acetic acid, incubated for 30 min at 60° C. and another 4 hours at 25° C. and clarified using a separator/filter press. The process caused a significant reduction of bacterial protein and DNA in the resulting clarified liquid. dsRNA was quantified using HPLC method (described below).

The dsRNA was sequenced, and the sequence determined to be identical to SEQ ID NO: 87. dsRNA sequence, stem and loop structure are as described above.

dsRNA Quantification by HPLC

Analysis of dsRNA was performed using ion pair reverse phase chromatography (IP RP HPLC), according to the following parameters:

TABLE IV

| Column | Phenomenex, C18, 5 um 300 A, 4.6 × 250 mm, | | | | |
|---|---|---|---|---|---|
| | batch # 5243-0061 | | | | |
| Solvents | Solvent A: 0.1M TEAA | | | | |
| | (triethylammonium acetate) pH 7.0 | | | | |
| | Solvent B: 25% Acetonitrile | | | | |
| | (ACN) in 0.1M TEAA pH 7.0 | | | | |
| | Solvent C: 100% ACN | | | | |
| | Solvent D: 100% Water | | | | |
| Gradient | Time [min] | % A | % B | % C | % D |
| | 3 | 100 | 0 | 0 | 0 |
| | 10 | 65 | 35 | 0 | 0 |
| | 50 | 35 | 65 | 0 | 0 |
| | 55 | 35 | 65 | 0 | 0 |
| | 56 | 0 | 0 | 50 | 50 |
| | 66 | 0 | 0 | 50 | 50 |
| | 66 | 100 | 0 | 0 | 0 |
| | 77 | 100 | 0 | 0 | 0 |

| | |
|---|---|
| Temperature | 25° C. |
| Flow rate | 1 ml/min |
| Detection | 260 nm |
| Injection | 40 μl |

### Reference Standard

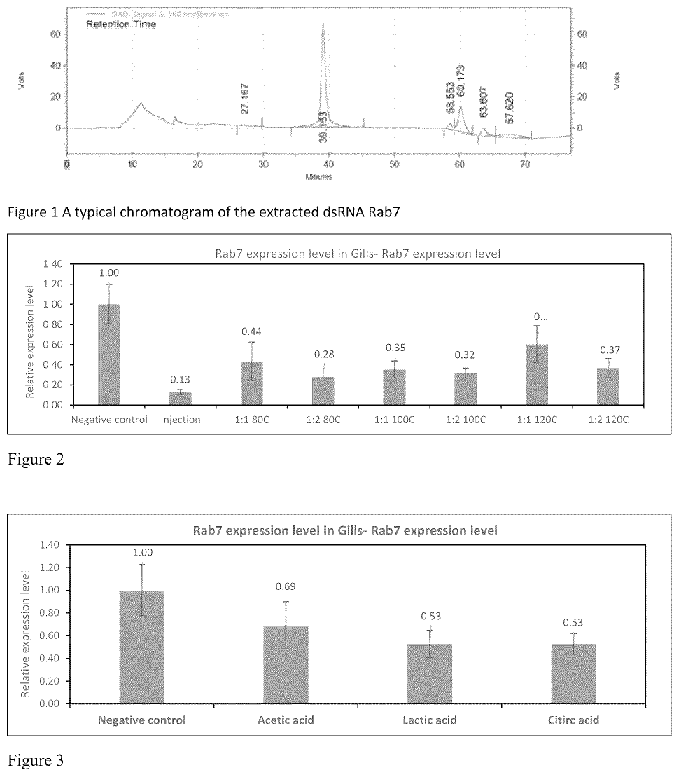

VP28 from Agro was used as a reference standard (FIG. 1). This sample was previously analyzed and quantified by gel and by HPLC (using a calibration curve) and its concentration of VP28 was estimated as 260-270 ppm (ng/ul). The non-diluted sample and 2-fold diluted sample (260 and 130 ppm respectively) were analyzed by HPLC. Samples with a concentration of 130 ppm were analyzed several times during the HPLC sequence. The area peaks of this sample were averaged and used to quantify the unknown samples.

### Synthesis of Chitosan-dsRNA Particles

Chitosan (chitosan, cat no. GP5480, greater than or equal to 90% deacetylated, very low molecular weight-approx. 30,000 daltons) was dissolved in 0.2M citric, lactic or sodium acetate buffer (pH 3-4) at a concentration of 3 mg/ml. dsRNA extract described above was diluted to the desired concentration using DDW. Chitosan-dsRNA particles were prepared by mixing equal volumes of dsRNA and chitosan solutions. Particles of distinct Chitosan:RNA ratios were synthesized by adjusting the concentration of chitosan.

In order to test the effect of Chitosan:RNA ratios on resulting particle characteristics, i.e., size distribution, zeta potential, RNAase tolerance, and solubility were measured for particles with Chitosan:RNA ratios of 4:1, 1:1, 0.5:1, 0.25:1.

### Particle Size Distribution

Complex particle size distribution was determined using a Mastersizer 2000 Laser Diffraction Particle Size Analyzer (Malvernpananalytical, Malvern, UK)

### Zeta Potential

Zeta potential of the synthesized particles was measured using a Malvern Zetasizer (Malvernpananalytical, Malvern, UK). A positive zeta potential indicates the negatively charged dsRNA molecules are sterically contained within the positively charged Chitosan molecules. A negative zeta potential indicate the opposite sterical conformation, suggesting the dsRNA would be exposed. Particle size measurements were performed at a 173° angle and a temperature of 25° C. The size is expressed as the z-average hydrodynamic diameter obtained by a cumulative analysis of the correlation function using the viscosity and refractive index of water in the calculations.

### Particle Retention Assay

After particle formation, dsRNA retention within the particle was assessed by gel electrophoresis in 2% agarose (Sigma) in TAE (X1, Hy-Labs) with ethidium bromide (Hy-Labs). Gels were run at 100 mV for 30 min and dsRNA retention was visualized under UV light.

Solubility of the Complexed dsRNA

In order to assess the solubility in water of the complexed dsRNA, the complex solution was centrifuged at 17000×g for 5 min and the resulting supernatant was loaded onto the gel described above to evaluate the presence of dsRNA is the aqueous phase.

### RNAse Tolerance Assay

Complexed dsRNA was assayed for susceptibility to enzymatic digestion using the RNAse A enzymatic degradation assay. Briefly, free dsRNA and Chitosan/dsRNA complexes were first exposed to RNAse A (Promega, Cat #A7970, Madison, WI) for an hour at 37° C. (1 μg RNAse A per 1 mg complexed RNA). The dsRNA was released from the complex by incubation of 40 min at 37° C. in a 0.1M Borax buffer pH 8.8. Following release from the complex, samples were analysed using 2% Agarose gel electrophoresis.

### Oral Delivery to Shrimp

Following the characteristics of complexes and feed, the various feed prepared was administrated to shrimp according to the following experiment design: 15 shrimp (Litopenaeus vannamei) per tank, at PL64 (chronological age—64 days after completing the metamorphosis period to post larvae) with an average weight of 240 mg. 3 tanks were assigned for each test article, except injection control and the negative control that had 1 tank per treatment. Tissues (whole shrimp) were collected from 5 shrimp per tank after 2, 4 and 5 days from the experiment start and at least after 18 hours from the last feed.

### Feeding:

Feeding during the experiment was administered twice a day, for up to 5 days. Feeding was calculated according to 20% of the body weight (BW) per day. The amount of pellets remaining after feeding was recorded.

### Controls:

The controls in the feeding experiment were negative control, in which the shrimp were fed with feed that did not contain any dsRNA, but contained an amount of chitosan very similar or equal to the amount provided in the chitosan:RNA particles.

### Water Parameters:

Water parameters were recorded and adjusted during the experiment. Values were recorded for temperature, ammonia concentration, nitrite and the salinity percent.

### Sample Collection:

Gills from 4 shrimp from each tank were collected after 2, 4 or 5 days and at least 18 hours after the last feeding into 1.5 ml collection tubes, containing ˜10×1-1.6 Φmm glass beads. The collection tubes were immediately frozen in liquid nitrogen and stored in −80° C. until further processing.

### RNA Extraction:

RNA extraction was performed using Trizol Reagent (LIFE TECHNOLOGIES EUROPE BV, The Netherlands), according to the manufacturer's instruction. Following the extraction, the RNA concentration was measured spectrophotometrically using a Nanodrop One (ThermoFisher) and visualized using 1.5% agarose gel to verify the RNA integrity.

### Gene Expression Analysis:

3000 ng (3 ug) of the RNA were treated with DNAse I (Sigma Aldrich), in order to remove any genomic DNA contaminants, then convert into cDNA using Thermo Scientific RevertAId RT kit (ThermoFisher), according the manufacturers instructions and random primers. Quantification of the Rab7 gene (Accession #FJ811529) by quantitative PCR was performed as described by (Vatanavicharn et al., 2014). The primers sequence for qRT analysis was selected from Rab7 fragment that is not part of the fragment chosen for the dsRNA production to avoid errors caused by the dsRNA delivery. 2 μl of the cDNA was used in a 10 μl reaction prepared using qPCRBIO Probe Mix Lo-ROX (PCR Biosystems) and PrimeTime qPCR Assays (IDT) containing the following primers and probe: Rab7 Forward GGGATACAGCTGGTCAAGAAA (SEQ ID NO: 88); Rab7 Reverse CGAGAGACTTGAAGGTATTGGG (SEQ ID NO: 89) and FAM labeled probe—CGAGGAGCTGATTGTTGTGTTCTCGT (SEQ ID NO: 90), (500 nM primers and 250 nM probe). PCR was performed in CFX96 Touch Real-Time PCR Detection System using the default thermal cycling conditions. Real-time RT-PCR Ct values obtained for Rab7 mRNA were normalized against Ct values obtained for EF1α mRNA (Accession #GU136229) using the following primers and probe: EF1a Forward GTGGAGACCTTCCAACAGTATG (SEQ ID NO: 91), EF1a Reverse CCTTCTTGTTGACCTCCTTGAT (SEQ ID NO: 92) and FAM labelled probe TGCGTGACATGAAGCAGACGG (SEQ ID NO: 93). A mean delta Ct value ±SD was determined for each treatment and the quantification was relative to a non-treated control that was set to 1.

### Example 1

dsRNA Quantification by HPLC
dsRNA Analysis Sample Preparation

The samples were diluted to an approximate concentration of 200 ppm and filtered using 0.20 μm or 0.45 μm. dsRNA concentration was calculated according to its area peak relative to reference standard area peaks. FIG. 1 shows a representative chromatogram of the extracted RNA.

### Particle Solubility Assay

To evaluate complex integrity and stability at different chitosan:dsRNA ratios, three complexes with ratios of 4:1, 1:1; 0.5:1 and 0.25:1 (w:w) chitosan:dsRNA were prepared as described in the methods section. Following complexation, 10 ul from each complex were loaded on Agarose gel and separated for 30 min at 100 mV. In addition, to evaluate the amount of non-complexed dsRNA, the complexes were centrifuged at 20,000 g for 10 min and 10 μl the supernatant was loaded on the gel.

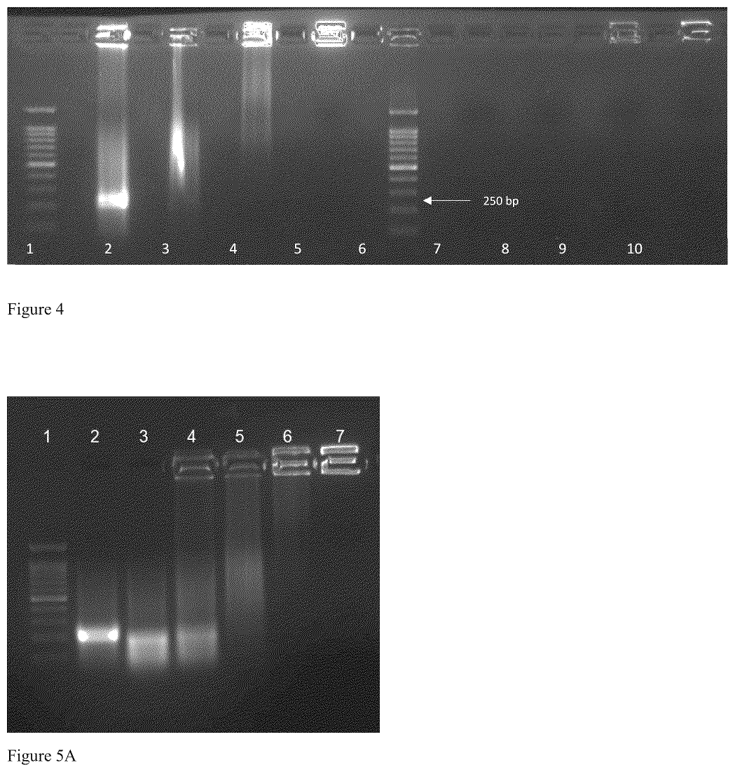

As can be seen from the photo of the gel in FIG. 4, complexes were generated using all 4 ratios, since no dsRNA was detected in the supernatant lanes (lanes 7-10). Without wishing to be bound by a single hypothesis, detection of a weak 250 bp dsRNA band lane 2 may imply that weak interactions are present between chitosan and dsRNA at a chitosan:dsRNA mass ratio of 0.25:1, resulting in release of dsRNA during separation on the Agarose gel.

### RNAase Tolerance

The ability of a carrier to protect its payload from nuclease degradation is an important property for efficient implementation of RNA silencing technology, to counteract nuclease digestion both during particle delivery and within the target cells. To address this, the degree of resistance to enzymatic degradation was assessed for the different complexes in the presence of RNase A solution (using an RNase A concentration capable of complete degradation of naked dsRNA within 30 min). To further characterize the stability of the three complexes produced by three different chitosan:dsRNA ratios and their ability to protect dsRNA from degradation, an RNase protection assay was performed. FIG. 5A shows a gel image of the resulted complexes after being subjected to the RNase treatment. As can be seen, free dsRNA and dsRNA in complex with chitosan at a chitosan:dsRNA mass ratio of 0.25:1 degrades to very low molecular weight following the treatment (FIG. 5A, lanes 3 and 4). In contrast no significant degradation is evident for the complexed dsRNA at chitosan:dsRNA ratios of 0.5:1, 1:1 and 4:1 (FIG. 5A, lanes 5-7). To further evaluate the integrity of the dsRNA inside the complex, the dsRNA was extracted from the complex as described hereinabove, and the naked dsRNA concentration was quantified by comparison to a known amount (FIG. 5B). As can be seen from the gel patterns, naked dsRNA was degraded completely following the RNase treatment but no degradation was observed from dsRNA extracted from the chitosan:RNA complex prepared using a mass ratio of 1:1. Comparison of the amounts of dsRNA extracted from the complexes shows that dsRNA extracted from complexes having mass ratios of 0.5:1, 1:1 and 4:1 is similar to the amount of dsRNA untreated, free dsRNA (FIG. 5B, lanes 2 and 5-7), while the amount of dsRNA extracted from complexes having a mass ratio of 0.25:1 is approximately 30% less than untreated dsRNA (FIG. 5B, lane 4). These results indicate that chitosan:RNA complexes of three mass ratios of 0.5:1, 1:1 and 4:1 can protect dsRNA from RNase degradation, while particles of complexes having a mass ratio of 0.25:1 have significantly lower interactions and are unable to adequately protect their nucleic acid (e.g. dsRNA) payload.

### Chitosan:RNA Particle Size Distribution

FIGS. 6A and 6B reveal that the mean particle size for the chitosan:RNA particle is a function of the complex's Chitosan:RNA ratio, but that particles of both ratios have a similar particle size distribution.

### Chitosan-dsRNA Powder Composition

TABLE V

| | Chitosan:dsRNA | Chitosan:dsRNA |
|---|---|---|
| Parameters | complex 1:1 | complex 1:2 |
| Size (dry after milling) | About 180 μm | About 180 μm |
| % H₂O | 4%-8% | 4%-8% |
| % dsRNA | 45% | 60% |
| % Chitosan | 45% | 30% |
| % Salts | 10% | 10% |

### Chitosan:RNA Particle Zeta Potential

Zeta potential of the above described complexes were measured using Zetasizer Nano ZSP (Malvern Instruments, UK).

Oral Delivery to Shrimp for Evaluation of dsRNA Bioavailability

Complexes described above, having chitosan:dsRNA mass ratios of 1:1 or 1:2 were further used to produce feed containing 2% dsRNA. To evaluate the effect of temperature on the particle-containing shrimp feed, the chitosan:dsRNA-containing shrimp feed was dried at different temperatures (80° C., 100° C., and 120° C.) after mixing.

Evaluation of bioavailability of the delivered dsRNA was performed by Rab7 gene expression analysis. The relative Rab7 expression results, summarized per RNA chitosan:dsRNA mass ratio and drying temperature as measured following 4 feeding days are shown in FIG. 2.

The results shown in FIG. 2 indicate that at all drying temperatures, a lower chitosan:dsRNA ratio (1:2) provides high bioavailability of the dsRNA to the shrimp cells through the oral route, even as compared to injection.

Anti-Viral Effect of Oral Delivery of dsRNA

In order to evaluate the efficacy of protection provided by gene silencing with oral delivery of the chitosan:dsRNA particles, shrimp feed was prepared with chitosan:dsRNA complexes 1:1 and 1:2 respectively, containing 0.5% dsRNA. Shrimp were fed the for 14 days and were challenged with the virus after 7 days of feeding. The rate of mortality was calculated after 14 days. Feeding the shrimp feed with the chitosan:dsRNA particles was effective in reducing viral mortality, with greater protection provided with particles having lower (1:2) chitosan:dsRNA mass ratio (Table VI).

TABLE VI
Virus challenge test - chitosan:dsRNA complex ratios 1:1 vs 1:2

| Chitosan:dsRNA complex ratio | % Mortality |
|---|---|
| 1:1 | 36.7 |
| 1:2 | 23.3 |

### Organic Acids—Effect on Complex Formulation and Bioavailability

Organic acids are known to have a beneficial effect on shrimp nutrition. To evaluate the role of different organic acids on the efficacy of dsRNA formulation and oral delivery, the effect of acetic, lactic and citric acids was tested by gene silencing using the dsRNA Rab7 model (FIG. 3) and by the viral challenge test, using VP 28 dsRNA (Table VII).

Lactic and citric acids have a better effect on gene silencing (Rab7 model) in comparison to acetic acid (FIG. 3). On the other hand, citric acid had a superior effect on the survival rates according to the viral challenge test (Table VII).

TABLE VII
Challenge test - the effect of organic acids

| Organic acid | % RPS |
|---|---|
| Acetic | 12.4 |
| Lactic | 37.5 |
| Citric | 56.3 |

Without wishing to be bound to a single hypothesis, the results may be attributed to the different pKa values of organic acids (Table VIII): Citric acid, having the lowest pKa value among the tested acids, proved to have the greatest anti-viral efficacy, suggesting that the stronger acid has a beneficial effect on dsRNA formulation and oral delivery to shrimps. Further, citric acid is a chelating agent, possibly contributing to dsRNA stabilization.

TABLE VIII
pKa values

| Compound | Formula | pKa |
|---|---|---|
| Acetic acid | CH₃CO₂H | 4.75 |
| Lactic acid | C₃H₆O₃ | 3.86 |
| Citric acid | C(OH)(CH₂CO₂H)₂CO₂H | 3.13 |

Chitosan:dsRNA Particle Stability

To evaluate the stability (“shelf life”) of chitosan:dsRNA particles prepared with citric acid under accelerated conditions, the complexes were incubated at 40° C. and 75% humidity for 3 months. No significant differences were observed between the two complexes implying that half amount of chitosan is sufficient to protect dsRNA from degradation, even under harsh storage conditions (Table IX).

TABLE IX
Stability trials: Chitosan:dsRNA mass ratio
1:1 vs Chitosan:dsRNA mass ratio 1:2

| Sample name | Time | Treatment | % dsRNA | dsRNA decrease (%) |
|---|---|---|---|---|
| Complex 1:1 in | T0 | Control | 34.66 | 0.00 |
| citric acid | T1 | Accelerated | 31.68 | 8.59 |
| | T3 | Accelerated | 30.70 | 11.44 |
| Complex 1:2 in | T0 | Control | 57.16 | 0.00 |
| citric acid | T1 | Accelerated | 52.54 | 8.08 |
| | T3 | Accelerated | 46.94 | 17.88 |

dsRNA was quantified using the HPLC method. Percent (%) dsRNA was calculated from the total complex weight.

Taken together, these results indicate that chitosan:RNA particles prepared according to the methods of the present invention using citric acid and having low chitosan:RNA mass ratios are effective in protecting and delivering active RNAi agents to the shrimps when mixed with feed for oral delivery, even after drying at high temperatures or long term storage.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. A particulate composition comprising chitosan and at least one type of RNA molecule comprising a nucleic acid sequence at least 50 bases in length, wherein said chitosan is 50-100% deacetylated low molecular weight chitosan, wherein the chitosan:RNA mass ratio (w/w) of said particle is in the range of 0.3-0.9, and wherein said RNA molecule is capable of silencing expression of a gene when administered to an organism expressing said gene, and comprising >50% particles in the range of 10-300 μm.

2. The particulate composition of claim 1, wherein said chitosan is 80-95% deacetylated low molecular weight chitosan.

3-7. (canceled)

8. The particulate composition of claim 1, wherein the molecular weight of said chitosan is about of 30 kDa.

9. The particulate composition of claim 1, wherein the chitosan:RNA ratio is in the range of 0.45-0.55 and the molecular weight of said chitosan is in the range of 20-30 kDa.

10-14. (canceled)

15. A nutriceutical composition comprising farmed crustacean food and the particulate composition of claim 1.

16. The nutriceutical composition of claim 15, wherein the RNA content (w/w) of said composition is in the range of 0.1-3.0%.

17-18. (canceled)

19. A method for producing a particulate chitosan-RNA composition comprising:
(i) rapidly adding an RNA solution to a volume of 50-100% deacetylated low molecular weight chitosan solution, wherein said chitosan solution comprises 2-20 mg/ml low molecular weight chitosan in 0.01-0.1 M citric acid,
(ii) desalting and concentrating the chitosan-RNA complex solution, and
(iii) drying the desalted, concentrated chitosan-RNA complex solution,
thereby producing the particulate chitosan-RNA composition.

20. The method of claim 19, wherein the chitosan solution of (i) comprises 2-20 mg/ml low molecular weight chitosan in 0.02 M citric acid, optionally 3 mg/ml low molecular weight chitosan in 0.02 M citric acid.

21. The method of claim 19, wherein said RNA solution comprises 0.2-10 mg/ml RNA in water, optionally 3 mg/ml RNA in water.

22. The method of claim 19, wherein said RNA solution is added to said chitosan solution in a ratio in the range of 1.0:0.14 to 1:1 volumes RNA:chitosan.

23-27. (canceled)

28. The method of claim 19, wherein said chitosan-RNA particles are a particulate composition comprising chitosan and at least one type of RNA molecule comprising a nucleic acid sequence at least 50 bases in length, wherein said chitosan is 50-100% deacetylated low molecular weight chitosan, wherein the chitosan:RNA mass ratio (w/w) of said particle is in the range of 0.3-0.9, and wherein said RNA molecule is capable of silencing expression of a gene when administered to an organism expressing said gene, and comprising >50% particles in the range of 10-300μ.

29. The nutriceutical composition of claim 15, wherein the particulate composition is prepared by:
(i) rapidly adding an RNA solution to a volume of 50-100% deacetylated low molecular weight chitosan solution, wherein said chitosan solution comprises 2-20 mg/ml low molecular weight chitosan in 0.01-0.1 M citric acid,
(ii) desalting and concentrating the chitosan-RNA complex solution, and
(iii) drying the desalted, concentrated chitosan-RNA complex solution.

30. A farmed crustacean comprising the particulate composition of claim 1.

31. (canceled)

32. A method of treatment or prevention of a disease or condition in a farmed crustacean associated with a pathogenic virus, the method comprising feeding the farmed crustacean the particulate composition of claim 1 or a nutriceutical composition comprising farmed crustacean food and the particulate composition of claim 1, wherein said at least one is targeted to a gene product of said pathological virus, and wherein ingestion of said particulate composition or nutraceutical composition by said farmed crustacean results in reduction in the level of a pathological virus in said farmed crustacean, compared to the same farmed crustacean ingesting feed devoid of RNA targeted to a gene product of said pathological virus.

33. (canceled)

34. The method of claim 32, wherein said farmed crustaceans are selected from the group consisting of Shrimp, Prawns, Crabs, Lobsters and Crayfishes, optionally wherein said farmed crustaceans are shrimp or prawns.

35. (canceled)

36. The method of claim 32, wherein said virus is selected from the group consisting of White Spot Syndrome Virus (WSSV, Accession No. AF332093), Taura syndrome virus (TSV, Accession No. NC_003005), Yellow head virus (YHV, Accession No. FJ848673.1), Gill-associated virus (GAV, Accession No. NC_010306.1), Infectious hypodermal and hematopoietic necrosis virus (IHHNV, Accession No. NC_002190), Infectious myonecrosis virus (IMNV, Accession No. KR815474.1), White Tail Disease (Macrobrachium rosenbergii nodavirus, MrNV, Accession No. NC_005094.1 (segment 1) and NC_005095.1 (segment 2)), Infectious pancreatic necrosis virus (IPNV, Accession No. NC_001915.1 (Segment A) and NC_001916.1 (Segment B)) and Decapod iridescent virus 1 (DIV1, Accession no. MF599468).

37. The particulate composition of claim 1, and/or a nutriceutical composition comprising farmed crustacean food and the particulate composition of claim 1, wherein said at least one type of RNA molecule comprises one or more of the following:
(a) at least one sequence capable of binding through complementary base pairing to a target mRNA molecule of a virus pathogenic in farmed crustaceans;
(b) at least one sequence having at least 90% sequence identity to a target mRNA molecule of a virus pathogenic in farmed crustaceans;
(c) at least one sequence at least partially complementary to a target mRNA molecule of a virus pathogenic in farmed crustaceans;
(d) at least one sequence identical to at least 21 contiguous bases of a target mRNA molecule of a virus pathogenic in farmed crustaceans;
(e) at least one sequence capable of binding through complementary base pairing to a target mRNA molecule of a farmed crustacean;
(f) at least one sequence identical to at least 21 contiguous bases of a target mRNA molecule of a farmed crustacean;
(g) at least one sequence at least partially complementary to a target mRNA molecule of a farmed crustacean.

38-39. (canceled)

40. The particulate composition and/or nutriceutical composition of claim 37, wherein said RNA molecule comprising at least one sequence capable of binding through complementary base pairing to a target mRNA molecule of a virus pathogenic in farmed crustaceans or comprising at least one sequence at least partially complementary to a target mRNA molecule of a virus pathogenic in farmed crustaceans comprises:
(a) a nucleic acid sequence complementary to a nucleic acid sequence selected from the group consisting of SEQ ID NO: 1, 2, 3 and 5.

41. The particulate composition and/or nutriceutical composition of claim 37, wherein said RNA molecule comprising at least one sequence having at least 90% sequence identity to a target mRNA molecule of a virus pathogenic in farmed crustaceans or at least one sequence identical to at least 21 contiguous bases of a target mRNA molecule of a virus pathogenic in farmed crustaceans comprises:
(a) a nucleic acid sequence identical to at least 21 contiguous bases of a nucleic acid sequence selected from the group consisting of SEQ ID NO: 59, 64, 77, 78 and 79, or
(b) a nucleic acid sequence complementary to a nucleic acid sequence selected from the group consisting of SEQ ID NOs: 59, 64, 77, 78 and 79.

42. The particulate composition and/or nutriceutical composition of claim 37, comprising at least one additional RNA sequence capable of directing cleavage of a target mRNA molecule of a virus pathogenic in farmed crustaceans.
